(19) United States
(12) Patent Application Publication
(10) Pub. No.: US 2025/0161308 A1
(43) Pub. Date: May 22, 2025

(54) Methods of Treating Covid-Related Disorders

(71) Applicant: Athira Pharma, Inc., Bothell, WA (US)

(72) Inventors: Hans J. Moebius, Wollerau (CH); Leen Kawas, Lynnwood, WA (US); Kevin Church, Mountlake Terrace, WA (US); Robert Taylor, Seattle, WA (US); Jewel Johnston, Seattle, WA (US); Douglas Boatman, Portland, OR (US)

(73) Assignee: Athira Pharma, Inc., Bothell, WA (US)

(21) Appl. No.: 18/839,669

(22) PCT Filed: Feb. 22, 2023
(86) PCT No.: PCT/US2023/013592

§ 371 Date: Aug. 19, 2024

Related U.S. Application Data
(60) Provisional application No. 63/313,015, filed on Feb. 23, 2022.

(51) Int. Cl. A61K 31/519; A61K 38/05; A61P 11/00; A61P 25/28

(52) U.S. Cl. CPC A61K 31/519; A61K 38/05; A61P 11/00; A61P 25/28

(57) ABSTRACT

The present disclosure relates to a method of treating long-term sequelae of infection with SARS-CoV-2, also known as long COVID. More particularly, it discloses the method of treating long COVID, the method comprising administering to a patient in need thereof a therapeutically effective amount of a HGF/MET positive modulating agent.

### CROSS-REFERENCE TO RELATED APPLICATIONS

This application claims the benefit of priority of U.S. Provisional Application No. 63/313,015, filed Feb. 23, 2022, which is incorporated by reference herein in its entirety for any purpose.

### FIELD

The present disclosure relates to methods of treating the long-term sequelae of infection with SARS-CoV-2, also known as long COVID or PACS (post-acute Covid19 sequelae).

### BACKGROUND

The global pandemic due to SARS-CoV-2 has resulted in a steep epidemiological increase of depressive and anxiety disorders (Santomauro, 2021). In addition, SARS-CoV-2 infections have resulted in a high number of long-term sequelae after initial recovery from infection, both unspecific CNS syndromes (e.g., fatigue) and specific symptoms (e.g., depression, anxiety) (Paul et al., 2021).

Most psychiatric central nervous system (CNS) disorders have no identified direct neuroanatomical or functional brain correlate. In contrast, the pathophysiology of long-Covid CNS manifestations appears to concern the whole brain in a fundamental way, most likely via a generalized vasculitis and downstream processes (inflammation, hypo-perfusion, hypo-oxygenation). If severe, SARS-CoV-2 can also lead to delirium and psychosis, inflammatory syndromes (such as encephalitis and acute disseminated encephalomyelitis), ischemic and hemorrhagic stroke (Iadecola et al., 2020; Koralnik and Tyler, 2020). “Localized” manifestations such as post-Covid Guillain-Barre syndrome have also been described with long Covid (Raahimi et al., 2021).

The neurological syndrome which can become part of prolonged recovery from SARS-CoV2 infection has no rational, targeted therapy. All current attempts to therapy are off label, and while these therapies appear to target common clinical symptom(s), these therapies are not derived from a common pathophysiology, and are not causal in their mode of action.

Hepatocyte growth factor (HGF) is a pleiotropic protein factor involved in numerous biological processes including embryonic and organ development, regeneration, and inflammation. HGF is a critical contributor to cortical, motor, sensory, sympathetic, and parasympathetic neuronal development and maturation. HGF is translated and secreted as inactive pro-HGF, but following cleavage, the resultant α and β-subunits are joined by a disulfide linkage to form the active heterodimer. Expression of HGF predominantly occurs in mesenchymal cells such as fibroblasts, chondroblasts, adipocytes, and the endothelium. Expression has also been demonstrated in the central nervous system (CNS) including in neurons, astrocytes, and ependymal cells (Nakamura and Mizuno, 2010). All biological activities of HGF are mediated through MET, a transmembrane receptor tyrosine kinase that serves as the sole known receptor for HGF. MET has known involvement in a variety of biological processes, with demonstrated roles in development, regeneration, and response to injury. Upon binding of HGF to the extracellular domain of MET, homo-dimerization of the MET protein leads to auto-phosphorylation of the intracellular domain. Phosphorylation of MET intracellular domains leads to recruitment and phosphorylation of a variety of effector proteins including Gab1, GRB2, Phospholipase C, and Stat3 (Gherardi et al., 2012; Organ and Tsao, 2011). These effector proteins then interact with downstream signaling pathways including PI3K/Akt, Ras/Raf/MAPK, RAC1/CDC42, and RAP/FAK among others to influence an array of cellular components including gene regulation, cytoskeletal rearrangements, cell cycle progression, cell adhesion, survival, and proliferation (Organ and Tsao, 2011). Because HGF has a demonstrated role in development (Nakamura et al., 2011), homeostasis (Funakoshi and Nakamura, 2003), suppression of cell death, and regeneration (Matsumoto et al., 2014), stimulation of the hepatocyte growth factor/MET (HGF/MET) signaling system is an ideal target for therapeutics for a range of disease states. Therapeutics involving HGF activity modulation have been proposed for disease and injury in many diverse tissue types including liver, kidney, gastrointestinal tract, cardiovascular components, lung, skin, nervous system, and musculature (Matsumoto et al., 2014).

Growing evidence suggests that complex CNS disorders are unlikely to be caused by a single route of pathology; they are likely the result of a multifactorial interplay related to genetics, age, and environment. Pharmacological stimulation of the HGF/MET signaling system may stop neurodegeneration and promote neuro-regeneration. The neurotrophic factor system represents a promising therapeutic target, and drugs that stimulate these systems have the potential to address neurodegeneration and improve cognition by protecting existing neurons, promoting connectivity, inducing neuro-regenerative mechanisms, by decreasing inflammation and improving cerebral blood flow (Funakoshi, 2011). The therapeutic promise of neurotrophic factors in neurological disorders is hampered by the lack of efficient and non-invasive delivery to the brain.

Therefore, a small molecule approach capable of passing the blood brain barrier and entering all regions of the brain, presents a superior therapeutic strategy for targeting neurotrophic factors to treat long COVID.

### SUMMARY

The present invention provides, in some embodiments, methods of treating long COVID.
Embodiment 1. A method of treating long COVID, comprising administering to a patient in need thereof a therapeutically effective amount of a HGF/MET agonist.
Embodiment 2. A method of treating long-term sequelae of infection with SARS-CoV-2, comprising administering to a patient in need thereof a therapeutically effective amount of a HGF/MET agonist.
Embodiment 3. A method of treating post-acute COVID-19 syndrome, comprising administering to a patient in need thereof a therapeutically effective amount of a HGF/MET agonist.
Embodiment 4. A method of treating post-COVID-19 condition, comprising administering to a patient in need thereof a therapeutically effective amount of a HGF/MET agonist.
Embodiment 5. A method of improving event related potential (ERP) P300 latency in a patient diagnosed with long COVID, comprising administering to a patient in need thereof a therapeutically effective amount of a HGF/MET agonist.
Embodiment 6. A method of slowing the decline in cognition or improving cognition in a patient diagnosed with long COVID, comprising administering to a patient in need thereof a therapeutically effective amount of a HGF/MET agonist.
Embodiment 7. A method of slowing the decline in the ability to perform activities of daily living and verbal fluency or improving the ability to perform activities of daily living and verbal fluency in a patient diagnosed with long COVID, comprising administering to a patient in need thereof a therapeutically effective amount of a HGF/MET agonist.
Embodiment 8. A method of slowing the decline in functional or cognitive capacity in a patient diagnosed with long COVID, comprising administering to a patient in need thereof a therapeutically effective amount of a HGF/MET agonist.
Embodiment 9. A method of slowing clinical decline in a patient diagnosed with long COVID, comprising administering to a patient in need thereof a therapeutically effective amount of a HGF/MET agonist.
Embodiment 10. A method of improving executive memory function in a patient diagnosed with long COVID, comprising administering to a patient in need thereof a therapeutically effective amount of a HGF/MET agonist.
Embodiment 11. The method of any one of the preceding embodiments, wherein the HGF/MET agonist is an antibody, an aptamer, a peptide, and/or a small molecule.
Embodiment 12. The method of any one of the preceding embodiments, wherein the HGF/MET agonist is N-hexanoic-Tyr-Ile-(6)aminohexanoic amide (Dihexa) or a pharmaceutically acceptable salt or a prodrug thereof.
Embodiment 13. The method of any one of the preceding embodiments, wherein the HGF/MET agonist is

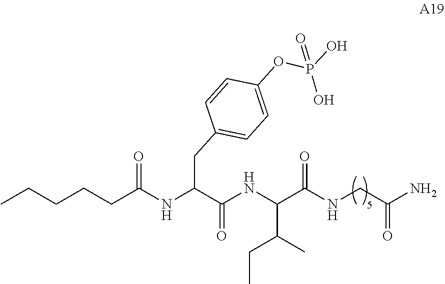

or a pharmaceutically acceptable salt thereof.
Embodiment 14. The method of any one of the preceding embodiments, comprising administering to the patient 2-90 mg per day of the HGF/MET agonist.
Embodiment 15. The method of any one of embodiments 1 to 11, wherein the HGF/MET agonist is
   a) a compound of Formula (I):

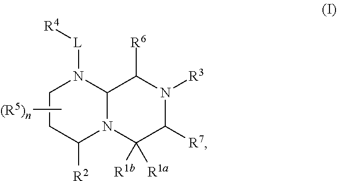

or a pharmaceutically acceptable salt thereof, wherein:
L is a direct bond, —C(═O)—, —(CR^{a}R^{b})ₘ—C(═O)—, —C(═O)—(CR^{a}R^{b})ₘ—, or —(CR^{a}R^{b})ₘ—;
each R^{a} and R^{b} is independently H, C₁-C₆ alkyl, C₂-C₆ alkenyl, or C₂-C₆ alkynyl;
R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl;
R² is H, oxo, or thioxo;
R³ is C₂-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl,
wherein the 5- to 10-membered heteroarylalkyl or 5- to 10-membered heterocyclylalkyl contains 1-3 heteroatoms selected from nitrogen and oxygen;
R⁴ is C₆-C₁₀ aryl, 5- to 10-membered heteroaryl, or 5- to 10-membered heterocyclyl,
wherein the 5- to 10-membered heteroaryl or 5- to 10-membered heterocyclyl contains 1-3 heteroatoms selected from nitrogen and oxygen;
each R⁵ is independently C₁-C₆ alkyl, oxo, or halo;
R⁶ is H, C₁-C₆ alkyl, or oxo;
R⁷ is H or oxo;
m is 1 or 2; and
n is an integer from 0 to 3;
wherein each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.
Embodiment 16. The method of embodiment 15, wherein the compound of Formula (I) is a compound of Formula (II), (IIa), (IIb), (IIc), (IId), or (IIe):

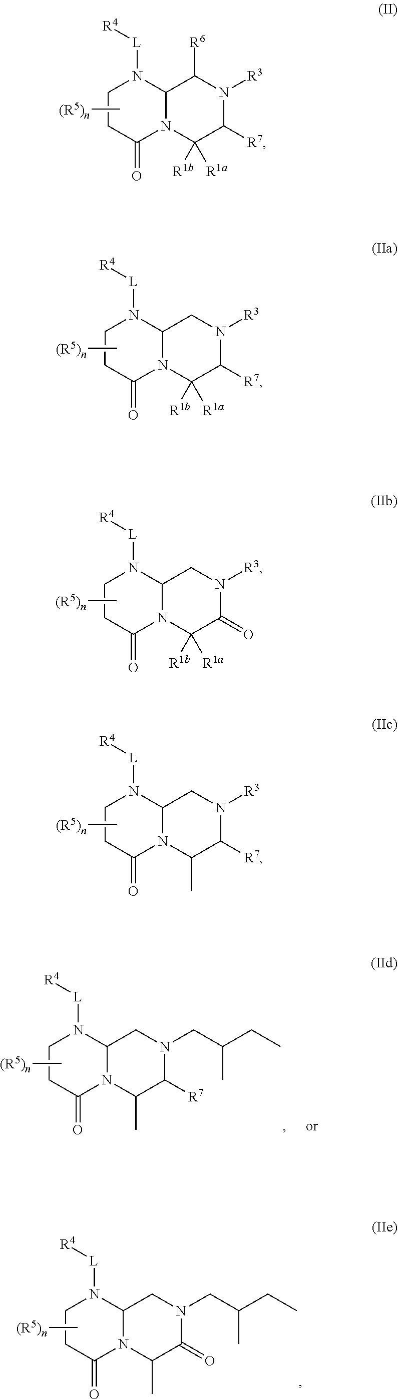

or a pharmaceutically acceptable salt thereof wherein L, R^{1a}, R^{1b}, R³, R⁴, R⁵, R⁶, R⁷, and n are as described for Formula (I).
Embodiment 17. The method of any one of embodiments 1 to 11 or 15 to 16, wherein the HGF/MET agonist is a Compound as set forth in Table 1, or a pharmaceutically acceptable salt thereof or a Compound as set forth in Table 1A, or a pharmaceutically acceptable salt thereof.
Embodiment 18. The method of any one of embodiments 1 to 11 or 15 to 17, wherein the HGF/MET agonist is

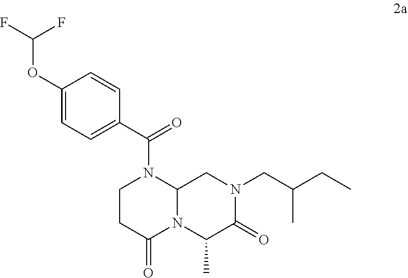 [SMILES: CCC(C)CN1CC2N(C(=O)c3ccc(OC(F)F)cc3)CCC(=O)N2[C@@H](C)C1=O]

a) or a pharmaceutically acceptable salt thereof.
Embodiment 19. The method of any one of embodiments 1 to 11 or 15 to 17, wherein the HGF/MET agonist is

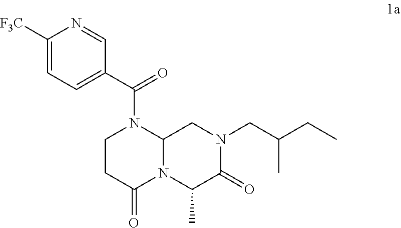 [SMILES: CCC(C)CN1CC2N(C(=O)c3ccc(C(F)(F)F)nc3)CCC(=O)N2[C@@H](C)C1=O]

or a pharmaceutically acceptable salt thereof.
Embodiment 20. The method of any one of the preceding embodiments, wherein prior to treatment, the patient exhibits one or more symptoms selected from anxiety, apathy, breathlessness, chest pain, cognitive impairment, depression, fatigue, impaired lung function, reduced liver function, increased rate of venous thromboembolism, muscle weakness, idiopathic pulmonary fibrosis, and/or respiratory complications.
Embodiment 21. The method of any one of the preceding embodiments, wherein prior to treatment, the patient exhibits cognitive impairment.
Embodiment 22. The method of embodiment 21, wherein the cognitive impairment is an attention, working memory, processing speed, executive functioning, phonemic fluency, category fluency, memory encoding, memory recall, and/or memory recognition cognitive impairment.
Embodiment 23. The method of embodiment 21 or embodiment 22, wherein the cognitive impairment suggests an executive pattern.
Embodiment 24. The method of any one of embodiments 21 to 23, wherein the cognitive impairment suggests a dysexecutive syndrome.
Embodiment 25. The method of any one of the preceding embodiments, wherein the patient has previously been diagnosed with airways disease, asthma such as moderate to severe asthma, bronchiectasis, bronchopulmonary dysplasia, cancer, chronic lung disease, chronic obstructive pulmonary disease (COPD), diabetes, hypertension, interstitial lung disease, pulmonary embolism, and/or pulmonary hypertension.
Embodiment 26. The method of any one of the preceding embodiments, which slows the decline in functional or cognitive capacity in the patient.
Embodiment 27. The method of any one of the preceding embodiments, which slows the decline in cognition in the patient.
Embodiment 28. The method of any one of the preceding embodiments, which improves cognition in the patient.
Embodiment 29. The method of any one of the preceding embodiments, which slows the decline in the ability to perform instrumental activities of daily living, basic activities of daily living, financial capacity, and/or verbal fluency in the patient.
Embodiment 30. The method of any one of the preceding embodiments, which improves the ability to perform activities of daily living, financial capacity, and/or verbal fluency in the patient.
Embodiment 31. The method of any one of the preceding embodiments, wherein the slowing of the decline or the improvement is determined after administering the treatment for at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks.
Embodiment 32. The method of any one of the preceding embodiments, which reduces the rate of decline, stabilizes, or improves an Instrumental (23-item) Activities of Daily Living (ADL) score.
Embodiment 33. The method of embodiment 32, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 34. The method of embodiment 32 or embodiment 33, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 35. The method of any one of the preceding embodiments, which reduces the rate of decline, stabilizes, or improves a Basic (19-item) Activities of Daily Living (ADL) score.
Embodiment 36. The method of embodiment 35, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 37. The method of embodiment 35 or embodiment 36, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 38. The method of any one of the preceding embodiments, which reduces the rate of decline, stabilizes, or improves a Financial Capacity Instrument (FCI) score.
Embodiment 39. The method of embodiment 38, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 40. The method of embodiment 38 or embodiment 39, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 41. The method of any one of the preceding embodiments, wherein the patient has a Mini-Mental State Examination (MMSE) score of less than or equal to 27 prior to the start of treatment.
Embodiment 42. The method of any one of the preceding embodiments, which reduces the rate of decline, stabilizes, or improves an MMSE score.
Embodiment 43. The method of embodiment 42, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 44. The method of embodiment 42 or embodiment 43, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 45. The method of any one of the preceding embodiments, wherein cognitive capacity is assessed by determining the patient's score before and after administration of the treatment using an Alzheimer's Disease (AD) Assessment Scale-Cognitive Subscale (ADAS-Cog).
Embodiment 46. The method of embodiment 45, wherein cognitive capacity is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 47. The method of embodiment 45 or embodiment 46, which reduces the rate of decline, stabilizes, or improves ADAS-Cog.
Embodiment 48. The method of any one of the preceding embodiments, which reduces the rate of decline, stabilizes, or improves a Montreal Cognitive Assessment test (MoCA) score.
Embodiment 49. The method of embodiment 48, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 50. The method of embodiment 48 or embodiment 49, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 51. The method of any one of the preceding embodiments, which reduces the rate of decline, stabilizes, or improves a Digit Symbol Substitution Test (DSST) score.
Embodiment 52. The method of embodiment 51, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 53. The method of embodiment 51 or embodiment 52, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 54. The method of any one of the preceding embodiments, which reduces the rate of decline, stabilizes, or improves a Disability Assessment for Dementia (DAD) score.
Embodiment 55. The method of embodiment 54, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 56. The method of embodiment 54 or embodiment 55, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 57. The method of any one of the preceding embodiments, which reduces the rate of decline, stabilizes, or improves a neuropsychiatric inventory (NPI) score.
Embodiment 58. The method of embodiment 57, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 59. The method of embodiment 57 or embodiment 58, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 60. The method of any one of the preceding embodiments, which reduces the rate of decline, stabilizes, or improves a Controlled Oral Word Association Test (COWAT) score.
Embodiment 61. The method of embodiment 60, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 62. The method of embodiment 60 or embodiment 61, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 63. The method of any one of the preceding embodiments, which reduces the rate of decline, stabilizes, or improves a Number Span forward and/or backward score.
Embodiment 64. The method of embodiment 63, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 65. The method of embodiment 63 or embodiment 64, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 66. The method of any one of the preceding embodiments, which reduces the rate of decline, stabilizes, or improves a Trail Making Test Part A and/or Part B score.
Embodiment 67. The method of embodiment 66, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 68. The method of embodiment 66 or embodiment 67, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 69. The method of any one of the preceding embodiments, which reduces the rate of decline, stabilizes, or improves a phonemic and category fluency score.
Embodiment 70. The method of embodiment 69, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 71. The method of embodiment 69 or embodiment 70, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 72. The method of any one of the preceding embodiments, which reduces the rate of decline, stabilizes, or improves a Hopkins Verbal Learning Test-Revised score.
Embodiment 73. The method of embodiment 72, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 74. The method of embodiment 72 or embodiment 73, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 75. The method of any one of the preceding embodiments, which provides fast improvement or normalization of P300 amplitude values.
Embodiment 76. The method of any one of the preceding embodiments, which provides fast improvement or normalization of event-related potential (ERP) P300 latency values.
Embodiment 77. The method of any one of the preceding embodiments, which provides fast improvement or normalization of ERP P300 latency values with some maintenance of effect at 4 weeks after discontinuation of treatment.
Embodiment 78. The method of any one of the preceding embodiments, which provides fast improvement or normalization of ERP P300 latency values, which is maintained at 4 weeks after discontinuation of treatment.
Embodiment 79. The method of any one of the preceding embodiments, which improves event-related potential (ERP) P300 latency.
Embodiment 80. The method of any one of embodiments 75 to 79, wherein the improvement or normalization of P300 values occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.
Embodiment 81. The method of any one of the preceding embodiments, wherein the HGF/MET agonist has an acceptable safety and tolerability profile.
Embodiment 82. The method of any one of the preceding embodiments, wherein the HGF/MET agonist is generally safe and well tolerated.
Embodiment 83. The method of any one of the preceding embodiments, wherein the HGF/MET agonist is administered by subcutaneous injection or by oral dosage, such as by solid oral dosage form.
Embodiment 84. The method of any one of the preceding embodiments, wherein the HGF/MET agonist is administered by subcutaneous injection.
Embodiment 85. The method of any one of the preceding embodiments, comprising administering the HGF/MET agonist for 26 weeks or more.
Embodiment 86. The method of any one of the preceding embodiments, wherein the HGF/MET agonist is a sodium salt of the compound of formula A19.
Embodiment 87. The method of any one of the preceding embodiments, wherein the HGF/MET agonist is a monosodium salt of the compound of formula A19.

### DETAILED DESCRIPTION

### Definitions and General Parameters

As used in the present specification, the following terms and phrases are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In some embodiments, the compounds of the present disclosure can be in the form of a “prodrug.” The term “prodrug” is defined in the pharmaceutical field as a biologically inactive derivative of a drug that upon administration to the human body is converted to the biologically active parent drug according to some chemical or enzymatic pathway. Examples of prodrugs include esterified carboxylic acids.

The compounds of the present disclosure can be in the form of a pharmaceutically acceptable salt. The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts, in particular their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine, amino acids, or other bases known to persons skilled in the art. The compounds of the present disclosure which contain one or more basic groups, i.e., groups which can be protonated, can be present and can be used according to the disclosure in the form of their addition salts with inorganic or organic acids.

The present disclosure provides pharmaceutical compositions comprising a compound of the present disclosure, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure can encompass any composition made by admixing at least one compound of the present disclosure and a pharmaceutically acceptable carrier.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, PA 17th Ed. (1985); and Modern Pharmaceutics, Marcel Dekker, Inc. 3rd Ed. (G. S. Banker & C. T. Rhodes, Eds.).

The terms “therapeutically effective amount” and “effective amount” are used interchangeably and refer to an amount of a compound that is sufficient to effect treatment as defined herein, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutically acceptable salt thereof for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

“Amino” refers to the —NH₂ radical.

“Carboxy” or “carboxyl” refers to the —CO₂H radical.

“Cyano” refers to the —CN radical.

“Hydroxy” or “hydroxyl” refers to the —OH radical.

“Nitro” refers to the —NO₂ radical.

“Oxo” refers to the ═O substituent.

“Thioxo” refers to the ═S substituent.

“Thiol” refers to the —SH substituent.

“Alkyl” refers to an unbranched or branched saturated hydrocarbon chain radical consisting solely of carbon and hydrogen atoms, having from one to twelve carbon atoms (C₁-C₁₂ alkyl), preferably one to eight carbon atoms (C₁-C₈ alkyl), one to six carbon atoms (C₁-C₆ alkyl), or one to three carbon atoms (C₁-C₃ alkyl) and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. Unless stated otherwise specifically in the specification, an alkyl group is optionally substituted.

“Alkenyl” refers to an unbranched or branched unsaturated hydrocarbon chain radical consisting solely of carbon and hydrogen atoms, which contains one or more carbon-carbon double bonds, having from two to twelve carbon atoms (C₂-C₁₂ alkenyl), preferably two to eight carbon atoms (C₂-C₅ alkenyl) or two to six carbon atoms (C₂-C₆ alkenyl), and which is attached to the rest of the molecule by a single bond, e.g., ethenyl, prop-1-enyl, but-1-enyl, pent-1-enyl, penta-1,4-dienyl, and the like. Unless stated otherwise specifically in the specification, an alkenyl group is optionally substituted.

“Alkynyl” refers to an unbranched or branched unsaturated hydrocarbon chain radical consisting solely of carbon and hydrogen atoms, which contains one or more carbon-carbon triple bonds, having from two to twelve carbon atoms (C₂-C₁₂ alkynyl), preferably two to eight carbon atoms (C₂-C₅ alkynyl) or two to six carbon atoms (C₂-C₆ alkynyl), and which is attached to the rest of the molecule by a single bond, e.g., ethynyl, propynyl, butynyl, pentynyl, hexynyl, and the like. Unless stated otherwise specifically in the specification, an alkynyl group is optionally substituted.

“Alkoxy” refers to a radical of the formula —ORₐ where Rₐ is an alkyl radical as defined above containing one to twelve carbon atoms. Preferred alkoxy groups have one to six carbon atoms (i.e., C₁-C₆ alkoxy) or one to three carbon atoms (i.e., C₁-C₃ alkoxy) in the alkyl radical. Unless stated otherwise specifically in the specification, an alkoxy group is optionally substituted.

“Aromatic ring” refers to a cyclic planar portion of a molecule (i.e., a radical) with a ring of resonance bonds that exhibits increased stability relative to other connective arrangements with the same sets of atoms. Generally, aromatic rings contain a set of covalently bound co-planar atoms and comprise a number of π-electrons (for example, alternating double and single bonds) that is even but not a multiple of 4 (i.e., 4n+2 π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an aromatic ring includes all radicals that are optionally substituted.

“Aryl” refers to a carbocyclic ring system radical comprising 6 to 18 carbon atoms and at least one aromatic ring (i.e., C₆-C₁₈ aryl), preferably having 6 to 10 carbon atoms (i.e., C₆-C₁₀ aryl). For purposes of embodiments of this disclosure, the aryl radical is a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, phenyl, pleiadene, pyrene, and triphenylene. Unless stated otherwise specifically in the specification, an aryl group is optionally substituted.

“Arylalkyl” refers to a radical of the formula —R_{b}—R_{c} where R_{b} is an alkylene chain and Re is one or more aryl radicals as defined above, for example, benzyl, diphenylmethyl and the like. An arylalkyl group may contain a C₁-C₁₀ alkylene chain connected to a C₆-C₁₀ aryl radical (i.e., C₆-C₁₀ arylalkyl). Unless stated otherwise specifically in the specification, an arylalkyl group is optionally substituted.

“Cycloalkyl” refers to a stable non-aromatic monocyclic or polycyclic carbocyclic radical consisting solely of carbon and hydrogen atoms, which may include fused or bridged ring systems, having from three to fifteen carbon atoms (i.e., C₃-C₁₅ cycloalkyl), preferably having from three to ten carbon atoms (i.e., C₃-C₁₀ cycloalkyl) or three to six carbon atoms (i.e., C₃-C₆ cycloalkyl), and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Cycloalkyl also includes “spiro cycloalkyl” when there are two positions for substitution on the same carbon atom. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group is optionally substituted.

“Cycloalkylalkyl” refers to a radical of the formula —R_{b}—R_{c} where R_{b} is an alkylene chain and Re is one or more cycloalkyl radicals as defined above, for example, cyclopropyl, cyclobutyl, cyclopentyl, cyclohexyl, cycloheptyl, and cyclooctyl and the like. A cycloalkylalkyl group may contain a C₁-C₁₀ alkylene chain connected to a C₃-C₁₂ cycloalkyl radical (i.e., C₃-C₁₂ cycloalkylalkyl) or a C₁-C₁₀ alkylene chain connected to a C₃-C₆ cycloalkyl radical (i.e., C₃-C₆ cycloalkylalkyl). Unless stated otherwise specifically in the specification, a cycloalkylalkyl group is optionally substituted.

“Fused” refers to any ring structure described herein which is fused to an existing ring structure in the compounds of the disclosure. When the fused ring is a heterocyclyl ring or a heteroaryl ring, any carbon atom on the existing ring structure which becomes part of the fused heterocyclyl ring or the fused heteroaryl ring is replaced with a nitrogen atom.

“Halo” or “halogen” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. A preferred haloalkyl group includes an alkyl group having one to six carbon atoms and that is substituted by one or more halo radicals (i.e., C₁-C₆ haloalkyl). The halo radicals may be all the same or the halo radicals may be different. Unless stated otherwise specifically in the specification, a haloalkyl group is optionally substituted.

“Haloalkoxy” refers to a radical of the formula —ORₐ where Rₐ is a haloalkyl radical as defined herein containing one to twelve carbon atoms. A preferred haloalkoxy group includes an alkoxy group having one to six carbon atoms (i.e., C₁-C₆ haloalkoxy) or having one to three carbon atoms (C₁-C₃ haloalkoxy) and that is substituted by one or more halo radicals. The halo radicals may all be the same or the halo radicals may all be different. Unless stated otherwise specifically in the specification, a haloalkoxy group is optionally substituted.

“Heteroaryl” refers to an aromatic group (e.g., a 5-14 membered ring system) having a single ring, multiple rings, or multiple fused rings, with one or more ring heteroatoms independently selected from nitrogen, oxygen and sulfur. As used herein, heteroaryl includes 1 to 10 ring carbon atoms and 1 to 4 heteroatoms independently selected from nitrogen, oxygen and sulfur within the ring. Preferred heteroaryl groups have a 5- to 10-membered ring system containing one to four heteroatoms selected from nitrogen, oxygen, and sulfur (i.e., a 5- to 10-membered heteroaryl) and a 5- to 6-membered ring system containing one to four heteroatoms selected from nitrogen, oxygen, and sulfur (i.e., a 5- to 6-membered heteroaryl). For purposes of embodiments of this disclosure, the heteroaryl radical may be a monocyclic, bicyclic, tricyclic or tetracyclic ring system, which may include fused or bridged ring systems. Examples of heteroaryl groups include pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl and thiophenyl (i.e., thienyl). A heteroaryl may comprise one or more N-oxide (N—O—) moieties, such as pyridine-N-oxide. Unless stated otherwise specifically in the specification, a heteroaryl group is optionally substituted.

“Heteroarylalkyl” refers to a radical of the formula —R_{b}—R, where R_{b} is an alkylene chain and R, is one or more heteroaryl radicals as defined above. A heteroarylalkyl group may contain a C₁-C₁₀ alkylene chain connected to a 5- to 10-membered heteroaryl group (i.e., 5- to 10-membered heteroarylalkyl) or a C₁-C₁₀ alkylene chain connected to a 5- to 6-membered heteroaryl group (i.e., 5- to 6-membered heteroarylalkyl). Unless stated otherwise specifically in the specification, a heteroarylalkyl group is optionally substituted.

“Heterocyclyl” refers to a saturated or unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or spiro, and may comprise one or more oxo (C═O) or N-oxide (N—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 1 to 10 ring carbon atoms, 1 to 8 carbon atoms, 1 to 6 carbon atoms, or 1 to 4 carbon atoms, and 1 to 5 ring heteroatoms, 1 to 4 heteroatoms, 1 to 3 heteroatoms, or 1 to 2 heteroatoms independently selected from nitrogen, sulfur and oxygen. Preferred heterocyclyls have five to 10 members in the ring system including one to four heteroatoms selected from nitrogen and oxygen (i.e., 5- to 10-membered heterocyclyl) or five to eight members in the ring system including one to four heteroatoms selected from nitrogen and oxygen (i.e., 5- to 8-membered heterocyclyl). Examples of heterocyclyl groups include dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl and 1,1-dioxo-thiomorpholinyl. Unless stated otherwise specifically in the specification, a hetercyclyl group is optionally substituted.

“Heterocyclylalkyl” refers to a radical of the formula —R_{b}—R, where R_{b} is an alkylene chain and R, is one or more heterocyclyl radicals as defined above. A heterocyclylalkyl group may contain a C₁-C₁₀ alkylene chain connected to a 5- to 10-membered heterocyclyl radical (i.e., 5- to 10-membered heterocyclylalkyl) or a C₁-C₁₀ alkylene chain connected to a 5- to 8-membered heterocyclyl radical (i.e., 5- to 8-membered heterocyclylalkyl). Unless stated otherwise specifically in the specification, a heterocyclylalkyl group is optionally substituted.

In some embodiments, the term “substituted” as used herein means any of the above groups, or other substituents (e.g., C₁-C₆ alkyl, C₂-C₆ alkenyl, C₂-C₆ alkynyl, C₃-C₁₂ cycloalkyl, C₃-C₁₂ cycloalkylalkyl, aryl, and heteroaryl) wherein at least one hydrogen atom (e.g., 1, 2, 3, or all hydrogen atoms) is replaced by a bond to a non-hydrogen atom such as, but not limited to: a halogen atom such as F, Cl, Br, and I (i.e., “halo”); an oxygen atom in groups such as hydroxyl groups or alkoxy groups (e.g., alkoxy or haloalkoxy); a nitrogen atom in groups such as amines (e.g., —NH₂), amides (e.g., —(C═O)NH₂), and nitro; alkyl groups including one or more halogen, such as F, Cl, Br, and I (e.g., haloalkyl); and cyano.

It is understood that each choice for L, R^{1a}, R^{1b}, R², R³, R⁴, R⁵, R⁶, and R⁷ is optionally substituted as described above unless specifically stated otherwise, and provided that all valences are satisfied by the substitution. Specifically, each choice for L, R^{1a}, R^{1b}, R², R³, R⁴, R⁵, R⁶, and R⁷ is optionally substituted unless specifically stated otherwise, and provided such substitution results in a stable molecule (e.g., groups such as H and halo are not optionally substituted).

### HGF/MET Positive Modulators

Positive modulators (i.e., agonists) of HGF/MET are small, brain penetrant molecules that specifically enhance HGF/MET signaling in the presence of HGF. In distressed tissues, e.g., during inflammation, local tissue HGF concentrations rise. This localization effect leads to a targeting of HGF/MET positive modulators, such as ATH-1017, towards desired brain areas in need of elevated neurotrophic activity. Phosphorylation of the intracellular MET receptor sites leads to a cascade of intracellular effects, which in turn fight inflammation, promote perfusion and metabolic homeostasis, and give rise to axonal arborization and increased synaptic connections. Impaired or structurally reduced synaptic connections are known to result in impaired cognitive capacity, including but not limited to ability to concentrate, orientation in time and space, and executive memory functions. In addition, it is likely that this functional or structural synaptic disconnection syndrome contributes to Behavioral and Psychological Signs of Dementia (BPSD, which includes, e.g., depression and anxiety) and thus also contributes to the clinical CNS syndrome in long COVID.

HGF/MET positive modulators have been shown preclinically to saturate the target at clinically relevant exposures. In healthy volunteers and Alzheimer's Disease (AD) subjects, ATH-1017 was shown to increase gamma power and reduce prolonged ERP P300 latency (Hua, 2022). These results support blood brain barrier penetration and target engagement in humans.

### ATH-1017 and Related Compounds

ATH-1017 is an experimental treatment, formulated as a sterile solution for subcutaneous (SC) injection. ATH-1017 is a prodrug, which is rapidly converted to the active metabolite ATH-1001 (Dihexa; see US2014/0094413) in the plasma after SC injection. ATH-1017 was developed as a water-soluble prodrug of ATH-1001 to allow SC dosing in aqueous vehicles. The active drug ATH-1001 acts as a positive modulator of the hepatic growth factor (HGF) receptor and its tyrosine kinase, MET, receptor system. Central nervous system (CNS) MET expression is crucial in maintaining the healthy adult brain (Hawrylycz, 2015), and is reduced in AD, particularly in the hippocampus and prefrontal cortex (Hamasaki, 2014). The HGF/MET system presents a therapeutic target to treat neurodegeneration and restore cognitive function following infection with SARS-CoV-2.

ATH-1017 is a pharmaceutically acceptable salt of the compound having the formula of A19:

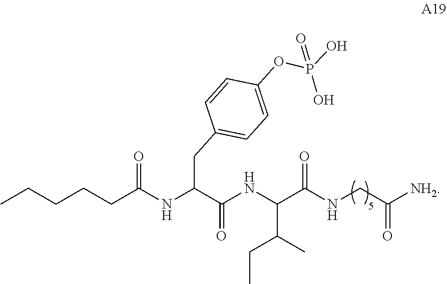

As used herein, and in the absence of a specific reference to a particular pharmaceutically acceptable salt and/or solvate of ATH-1017, any dosages, whether expressed in e.g. milligrams or as a % by weight, should be taken as referring to the amount of ATH-1017, i.e. the amount of:

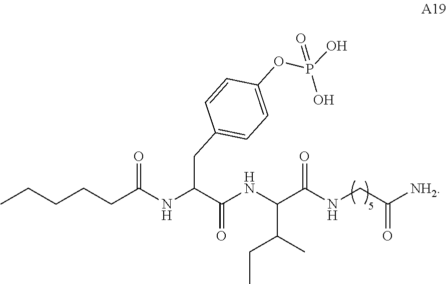

For example, therefore, a reference to “40 mg ATH-1017 or a pharmaceutically acceptable salt and/or solvate thereof” means an amount of ATH-1017 or a pharmaceutically acceptable salt and/or solvate thereof which provides the same amount of ATH-1017 as 40 mg of A19 free acid.

Nonlimiting exemplary pharmaceutically acceptable salts of A19 include:

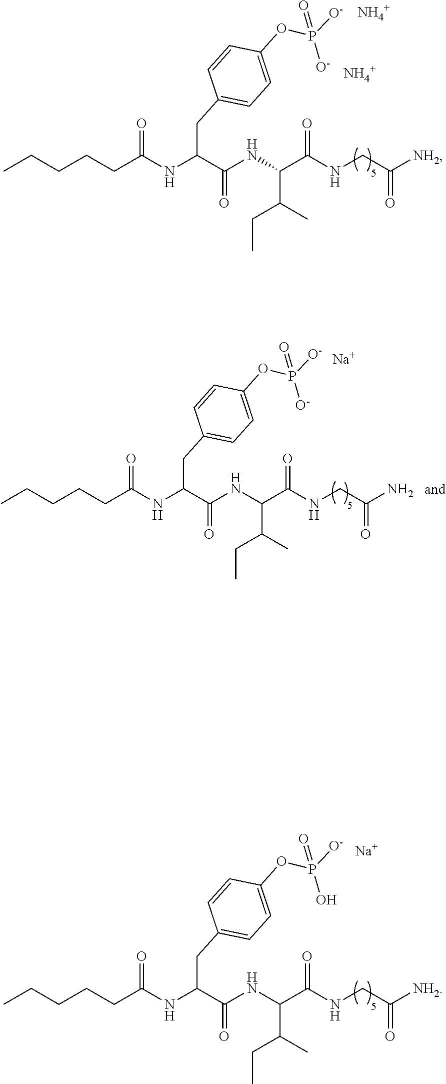

Unless otherwise indicated, ATH-1017 refers to the monosodium salt of A19, shown below:

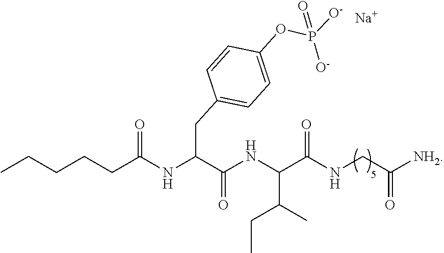

The compound of A19, and pharmaceutically acceptable salts thereof, including ATH-1017, may be synthesized and characterized using methods known to those of skill in the art, such as those described in PCT Publication No. WO 2017/210489 A1.

In some embodiments, ATH-1017 is formulated for subcutaneous administration. In some such embodiments, ATH-1017 is provided in a pre-filled syringe containing 1 mL of 40 mg/mL ATH-1017 or 70 mg/mL ATH-1017. In some embodiments, the ATH-1017 is in a solution comprising 10 mM sodium phosphate.

### Compounds of Formula (I)

Some embodiments provide uses of compounds of Formula (I):

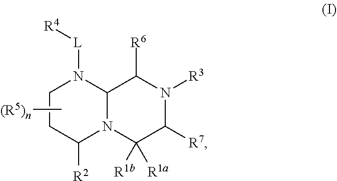

or a pharmaceutically acceptable salt thereof, wherein:
L is a direct bond, —C(═O)—, —(CR^{a}R_{b})ₘ—C(═O)—, —C(═O)—(CR^{a}R^{b})ₘ—, or —(CR^{a}R^{b})ₘ—;
each R^{a} and R^{b} is independently H, C₁-C₆ alkyl, C₂-C₆ alkenyl, or C₂-C₆ alkynyl;
R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl;
R² is H, oxo, or thioxo;
R³ is C₂-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl,
   wherein the 5- to 10-membered heteroarylalkyl or 5- to 10-membered heterocyclylalkyl contains 1-3 heteroatoms selected from nitrogen and oxygen;
R⁴ is C₆-C₁₀ aryl, 5- to 10-membered heteroaryl, or 5- to 10-membered heterocyclyl,
   wherein the 5- to 10-membered heteroaryl or 5- to 10-membered heterocyclyl contains 1-3 heteroatoms selected from nitrogen and oxygen;
each R⁵ is independently C₁-C₆ alkyl, oxo, or halo;
R⁶ is H, C₁-C₆ alkyl, or oxo;
R⁷ is H or oxo;
m is 1 or 2; and
n is an integer from 0 to 3;
wherein each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.

In some embodiments, L is a direct bond. In some embodiments, L is —C(═O)— or —(CR^{a}R^{b})ₘ—. In some embodiments, L is —C(═O)—. In some embodiments, L is —(CR^{a}R^{b})ₘ—. In some embodiments, L is —(CR^{a}R^{b})ₘ—C(═O)— or —C(═O)—(CR^{a}R^{b})ₘ—. In some embodiments, L is —(CR^{a}R^{b})ₘ—C(═O)—. In some embodiments, L is —C(═O)—(CR^{a}R^{b})ₘ—.

In some embodiments, each R^{a} and R^{b} is independently H, C₁-C₆ alkyl, C₂-C₆ alkenyl, or C₂-C₆ alkynyl. In some embodiments, each R^{a} and R^{b} is independently H, C₁-C₃ alkyl, C₂-C₄ alkenyl, or C₂-C₄ alkynyl. In some embodiments, R^{a} and R^{b} are each H. In some embodiments, R^{a} is H. In some embodiments, R^{a} is C₁-C₆ alkyl, such as methyl, ethyl, or propyl. In some embodiments, R^{a} is C₂-C₆ alkenyl, such as vinyl or propenyl. In some embodiments, R^{a} is C₂-C₆ alkynyl, such as ethynyl or propynyl. In some embodiments, R^{b} is H. In some embodiments, R^{b} is C₁-C₆ alkyl, such as methyl, ethyl, or propyl. In some embodiments, R^{b} is C₂-C₆ alkenyl, such as vinyl or propenyl. In some embodiments, R^{b} is C₂-C₆ alkynyl, such as ethynyl or propynyl.

In some embodiments, R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl. In some embodiments, R^{1a} is H. In some embodiments, R^{1a} is C₁-C₆ alkyl, such as methyl, ethyl, or propyl. In some embodiments, R^{1a} is C₂-C₆ alkenyl, such as vinyl or propenyl. In some embodiments, R^{1a} is C₂-C₆ alkynyl, such as ethynyl or propynyl. In some embodiments, R^{1a} is C₁-C₆ alkoxy, such as methoxy, ethoxy, or propoxy. In some embodiments, R^{1a} is halo, such as fluoro, chloro, or bromo. In some embodiments, R^{1a} is C₆-C₁₀ arylalkyl, such as benzyl. In some embodiments, R^{1b} is H. In some embodiments, R^{1b} is C₁-C₆ alkyl, such as methyl, ethyl, or propyl. In some embodiments, R^{1b} is C₂-C₆ alkenyl, such as vinyl or propenyl. In some embodiments, R^{1b} is C₂-C₆ alkynyl, such as ethynyl or propynyl. In some embodiments, R^{1b} is C₁-C₆ alkoxy, such as methoxy, ethoxy, or propoxy. In some embodiments, R^{1b} is halo, such as fluoro, chloro, or bromo. In some embodiments, R^{1b} is C₆-C₁₀ arylalkyl, such as benzyl.

In some embodiments, R^{1a} and R^{1b} are each independently H; C₁-C₆ alkyl optionally substituted with 1-3 substituents selected from halo, —CO₂H, and —C(═O)NH₂; C₁-C₆ alkoxy; halo; or C₆-C₁₀ arylalkyl optionally substituted by 1-3 substituents selected from halo and amino. In some embodiments, R^{1a} is C₁-C₆ alkyl substituted with 1-3 halo, such as fluoro or chloro. In some embodiments, R^{1a} is C₁-C₆ alkyl substituted with 1-3 —CO₂H groups. In some variations, R^{1a} is C₁-C₃ alkyl substituted with 1-2 CO₂H groups, such as —CH₂CO₂H or —CH₂CH₂CO₂H. In some embodiments, R^{1a} is C₁-C₆ alkyl substituted with 1-3 —C(═O)NH₂ groups. In some embodiments, R^{1a} is C₁-C₃ alkyl substituted with 1-2 —C(═O)NH₂ groups, such as —CH₂C(═O)NH₂ or —CH₂CH₂C(═O)NH₂. In some embodiments, R^{1a} is C₆-C₁₀ arylalkyl substituted by 1-3 substituents selected from halo and amino. In some embodiments, R^{1a} is C₆-C₁₀ arylalkyl substituted by 1-3 halo, such as fluoro, chloro, or bromo. In some embodiments, R^{1a} is C₆-C₁₀ arylalkyl substituted by 1-3 amino. In some embodiments, R^{1b} is C₁-C₆ alkyl substituted with 1-3 halo, such as fluoro or chloro. In some embodiments, R^{1b} is C₁-C₆ alkyl substituted with 1-3 —CO₂H groups. In some variations, R^{1b} is C₁-C₃ alkyl substituted with 1-2 CO₂H groups, such as —CH₂CO₂H or —CH₂CH₂CO₂H. In some embodiments, R^{1b} is C₁-C₆ alkyl substituted with 1-3 —C(═O)NH₂ groups. In some embodiments, R^{1b} is C₁-C₃ alkyl substituted with 1-2 —C(═O)NH₂ groups, such as —CH₂C(═O)NH₂ or —CH₂CH₂C(═O)NH₂. In some embodiments, R^{1b} is C₆-C₁₀ arylalkyl substituted by 1-3 substituents selected from halo and amino. In some embodiments, R^{1b} is C₆-C₁₀ arylalkyl substituted by 1-3 halo, such as fluoro, chloro, or bromo. In some embodiments, R^{1b} is C₆-C₁₀ arylalkyl substituted by 1-3 amino. In some embodiments, R^{1a} and R^{1b} are each independently H, methyl, fluoro, 2-methylbutyl, —CH₂F, methoxy, —CH₂CO₂H, —CH₂C(═O)NH₂, benzyl, or 4-aminobenzyl. In some embodiments, R^{1a} and R^{1b} are each independently H or C₁-C₃ alkyl. In some embodiments, R^{1a} is methyl and R^{1b} is H. In some embodiments, R^{1a} and R^{1b} are each H. In some embodiments, one of R^{1a} and R^{1b} is H and the other is C₁-C₃ alkyl, such as methyl.

In some embodiments, R² is H, oxo, or thioxo. In some embodiments, R² is H. In some embodiments, R² is oxo. In some embodiments, R² is thioxo.

In some embodiments, R³ is C₃-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl, wherein the 5- to 10-membered heteroarylalkyl or 5- to 10-membered heterocyclylalkyl contains 1-3 heteroatoms selected from nitrogen and oxygen. In some embodiments, R³ is C₃-C₆ alkyl, such as propyl, butyl, pentyl, or hexyl. In some embodiments, R³ is C₄-C₆ alkyl. In some embodiments, R³ is C₃-C₆ alkenyl. In some embodiments, R³ is C₄-C₆ alkenyl. In some embodiments, R³ is C₃-C₆ alkynyl. In some embodiments, R³ is C₄-C₆ alkynyl. In some embodiments, R³ is C₃-C₁₂ cycloalkyl, such as cyclopropyl, cyclobutyl, cyclopentyl, or cyclohexyl. In some embodiments, R³ is C₃-C₆ cycloalkyl. In some embodiments, R³ is C₃-C₆ cycloalkylalkyl, such as —(CH₂)₁₋₃(C₃-C₆ cycloalkyl). In some embodiments, R³ is C₆-C₁₀ arylalkyl, such as benzyl. In some embodiments, R³ is 5- to 10-membered heteroarylalkyl, such as —(CH₂)₁₋₃(5- to 10-membered heteroaryl) or —(CH₂)₁₋₃(5- to 6-membered heteroaryl). In some embodiments, the 5- to 10-membered heteroarylalkyl contains 1-2 nitrogen atoms. In some embodiments, R³ is 5- to 10-membered heterocyclylalkyl, such as —(CH₂)₁₋₃(5- to 10-membered heterocyclyl) or —(CH₂)₁₋₂(5- to 6-membered heterocyclyl). In some embodiments, the 5- to 10-membered heterocyclylalkyl contains 1-2 nitrogen atoms.

In some embodiments, R³ is C₃-C₆ alkyl optionally substituted by 1-3 substituents selected from halo and —C(═O)NH₂, C₂-C₆ alkenyl, or C₃-C₆ cycloalkylalkyl. In some embodiments, R³ is C₂-C₆ alkyl optionally substituted by 1-3 substituents selected from halo, C₁-C₃ alkoxy, hydroxy, —NH₂, —SO₂(C₁-C₃ alkyl), and —C(═O)NH₂; C₂-C₆ alkenyl; C₃-C₆ cycloalkylalkyl; 5- to 6-membered heteroarylalkyl; 5- to 6-membered heterocyclylalkyl; or C₆ arylalkyl. In some embodiments, R³ is C₂ alkyl substituted by 1-3 substituents selected from C₁—C₃ alkoxy, hydroxy, —NH₂, and —SO₂(C₁-C₃ alkyl). In some embodiments, R³ is:

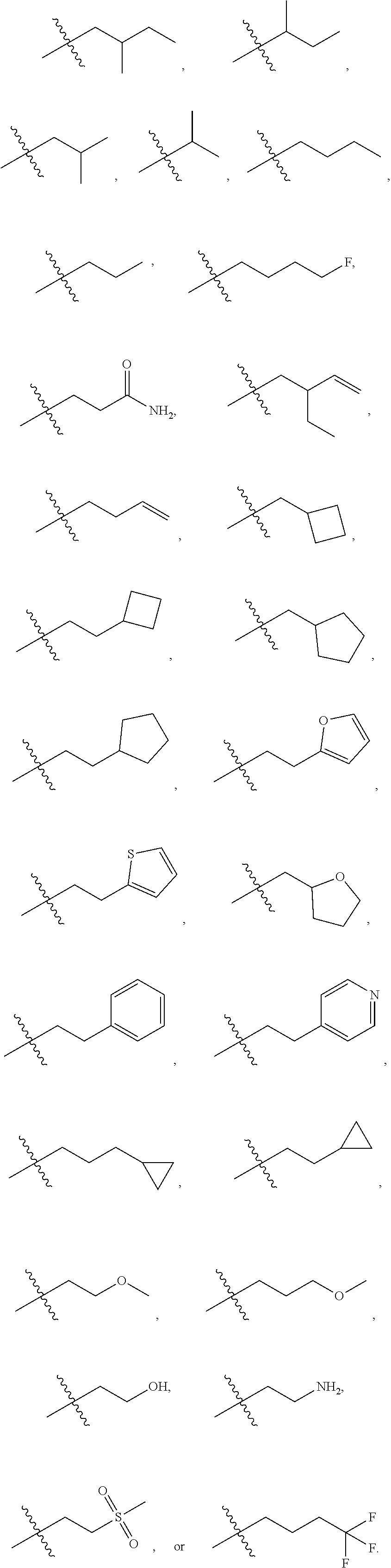

In some embodiments, R³ is:

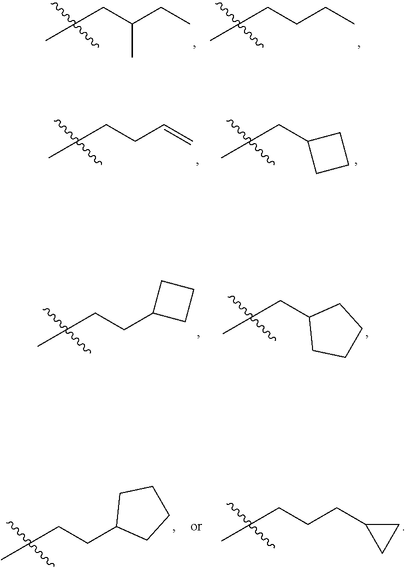

In some embodiments, R³ is 2-methylbutyl.

In some embodiments, R⁴ is C₆-C₁₀ aryl, 5- to 10-membered heteroaryl, or 5- to 10-membered heterocyclyl, wherein the 5- to 10-membered heteroaryl or 5- to 10-membered heterocyclyl contains 1-3 heteroatoms selected from nitrogen and oxygen. In some embodiments, R⁴ is C₆-C₁₀ aryl, such as phenyl. In some embodiments, R⁴ is 5- to 10-membered heteroaryl containing 1-2 nitrogen atoms. In some embodiments, R⁴ is 5- to 10-membered heterocyclyl. In some embodiments, R⁴ is 5- to 9-membered heterocyclyl containing 1-2 nitrogen atoms. In some embodiments, R⁴ is 5- to 9-membered heterocyclyl containing 1-2 oxygen atoms. In some embodiments, R⁴ is 5- to 9-membered heterocyclyl containing 1 nitrogen atom and 1 oxygen atom.

In some embodiments, R⁴ is C₆-C₁₀ aryl optionally substituted with 1-3 substituents selected from halo, hydroxyl, C₁-C₆ haloalkyl, and C₁-C₆ haloalkoxy. In some embodiments, R⁴ is phenyl substituted with 1-3 substituents selected from —CF₃, —OCHF₂, —OH, fluoro, and chloro. In some embodiments, R⁴ is:

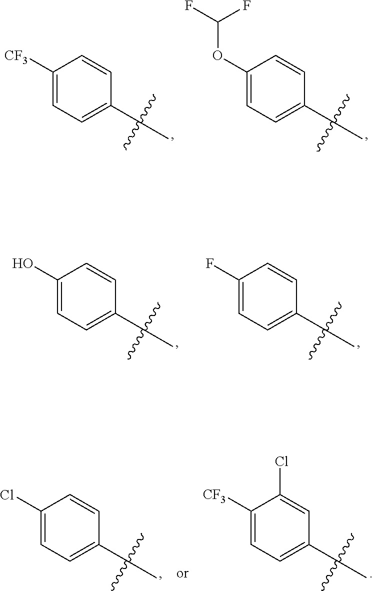 [SMILES: *c1ccc(C(F)(F)F)cc1 ; *c1ccc(OC(F)F)cc1 ; *c1ccc(O)cc1 ; *c1ccc(F)cc1 ; *c1ccc(Cl)cc1 ; *c1ccc(C(F)(F)F)c(Cl)c1]

In some embodiments, R⁴ is:

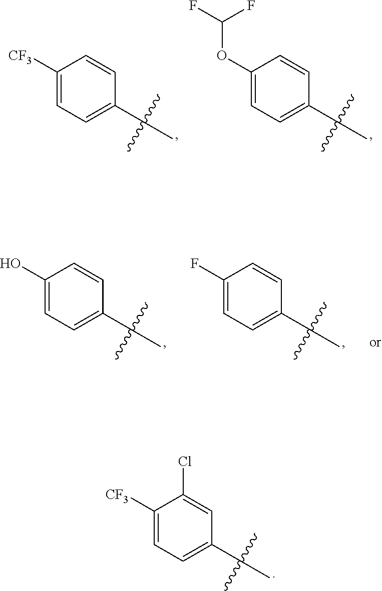 [SMILES: *c1ccc(C(F)(F)F)cc1 ; *c1ccc(OC(F)F)cc1 ; *c1ccc(O)cc1 ; *c1ccc(F)cc1 ; *c1ccc(C(F)(F)F)c(Cl)c1]

In some embodiments, R⁴ is 5- to 10-membered heteroaryl optionally substituted with 1-3 substituents selected from halo, hydroxyl, C₁-C₆ haloalkyl, and C₁-C₆ haloalkoxy. In some embodiments, R⁴ is pyridyl or indolyl optionally substituted with 1-3 substituents selected from halo, hydroxyl, C₁-C₆ haloalkyl, and C₁-C₆ haloalkoxy. In some embodiments, R⁴ is

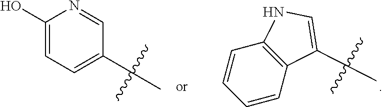 [SMILES: *c1ccc(O)nc1 ; *c1c[nH]c2ccccc12]

In some embodiments, R⁴ is pyridyl substituted with 1-3 substituents selected from halo, hydroxyl, C₁-C₆ haloalkyl, and C₁-C₆ haloalkoxy. In some embodiments, R⁴ is

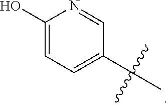 [SMILES: *c1ccc(O)nc1]

In some embodiments, R⁴ is 5- to 10-membered heterocyclyl optionally substituted with 1-3 substituents selected from halo, hydroxyl, C₁-C₆ haloalkyl, and C₁-C₆ haloalkoxy. In some embodiments, R⁴ is indolinyl.

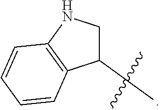 [SMILES: *C1CNc2ccccc21]

In some embodiments, -L-R⁴ is —CH₂(phenyl) or —C(O)(phenyl), wherein the phenyl is substituted by 1-3 substituents selected from C₁-C₃ haloalkyl, C₁-C₃ haloalkoxy, halo, and hydroxy. In some embodiments, -L-R⁴ is —CH₂(pyridyl) or —C(O)(pyridyl), wherein the pyridyl is substituted by 1-3 substituents selected from C₁-C₃ haloalkyl, C₁-C₃ haloalkoxy, halo, and hydroxy. In some embodiments, -L-R⁴ is:

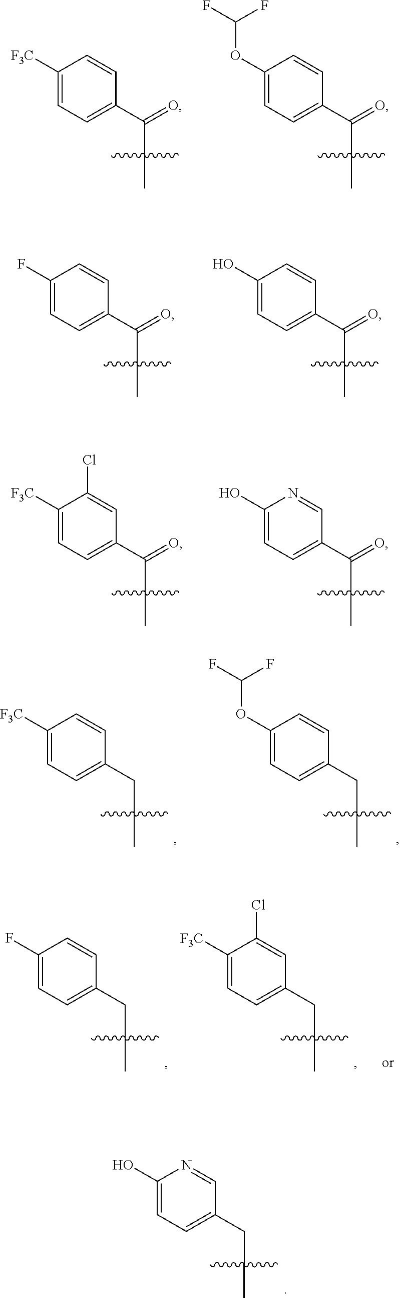

In some embodiments, each R⁵ is independently C₁-C₆ alkyl, oxo, or halo. In some embodiments, R⁵ is C₁-C₆ alkyl, such as methyl, ethyl, or propyl. In some embodiments, R⁵ is oxo. In some embodiments, R⁵ is halo, such as fluoro, chloro, or bromo. In some embodiments, R⁵ is oxo or halo. In some embodiments, R⁵ is oxo or fluoro.

In some embodiments, R⁶ is H, C₁-C₆ alkyl, or oxo. In some embodiments, R⁶ is H. In some embodiments, R⁶ is C₁-C₆ alkyl, such as methyl, ethyl, or propyl. In some embodiments, R⁶ is oxo.

In some embodiments, R⁷ is H or oxo. In some embodiments, R⁷ is H. In some embodiments, R⁷ is oxo.

In some embodiments, m is 1. In other embodiments, m is 2.

In some embodiments, n is 0. In other embodiments, n is an integer from 1 to 3. In some embodiments, n is 1. In some embodiments, n is 2. In some embodiments, n is 3.

In any embodiments of Formula (I), or variations thereof, each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to three substituents selected from hydroxyl, halo (such as fluoro, chloro, or bromo), amino, C₁-C₆ haloalkyl (such as —CF₃ or —CHF₂), C₁-C₆ alkoxy (such as methoxy or ethoxy), C₁-C₆ haloalkoxy (such as —OCHF₂ or —OCF₃), and —(C═O)NH₂.

In some embodiments, the compound of Formula (I) is a compound of Formula (II), (IIa), (IIb), (IIc), (IId), or (IIe).

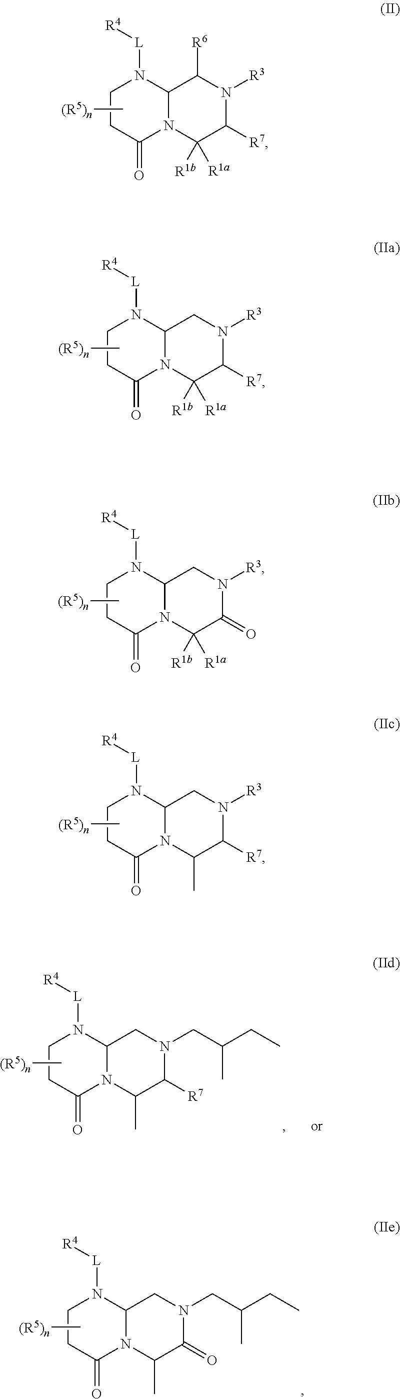

or a pharmaceutically acceptable salt thereof, wherein L, R^{1a}, R^{1b}, R³, R⁴, R⁵, R⁶, R⁷, and n are as described for Formula (I). In some embodiments, the compound is of Formula (II) or a pharmaceutically acceptable salt thereof. In some embodiments, the compound is of Formula (IIa) or a pharmaceutically acceptable salt thereof. In some embodiments, the compound is of Formula (IIb) or a pharmaceutically acceptable salt thereof. In some embodiments, the compound is of Formula (IIc) or a pharmaceutically acceptable salt thereof. In some embodiments, the compound is of Formula (IId) or a pharmaceutically acceptable salt thereof. In some embodiments, the compound is of Formula (IIe) or a pharmaceutically acceptable salt thereof.

In some embodiments, the compound of Formula (I) is a compound of Formula (IIIa), (IIIb), (IIIc), or (IIId):

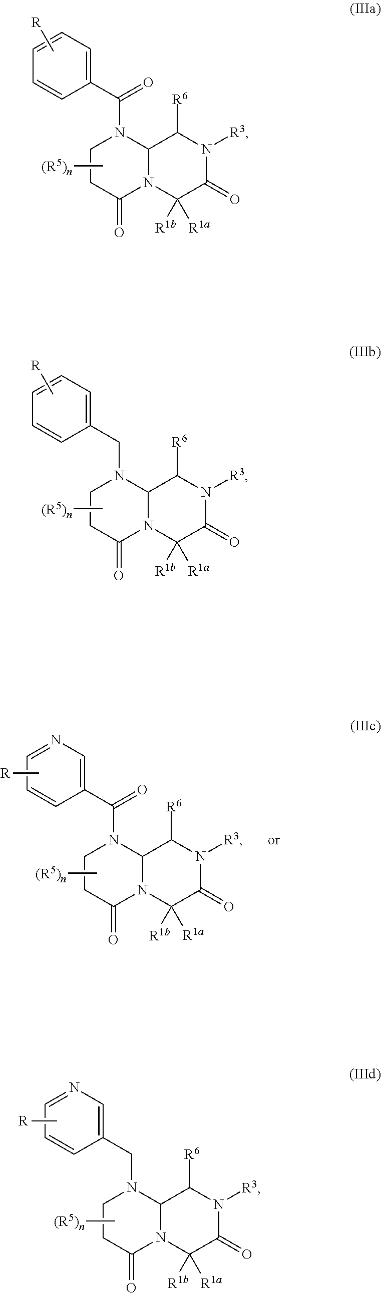

or a pharmaceutically acceptable salt thereof, wherein R^{1a}, R^{1b}, R³, R⁵, R⁶, and n are as described for Formula (I), and R represents one or more optional substituents, such as hydroxyl, halo, amino, C₁-C₆ haloalkyl, C₁-C₆ haloalkoxy, as described for Formula (I). In some embodiments, the compound is of Formula (IIIa) or a pharmaceutically acceptable salt thereof. In some embodiments, the compound is of Formula (IIIb) or a pharmaceutically acceptable salt thereof. In some embodiments, the compound is of Formula (IIIc) or a pharmaceutically acceptable salt thereof. In some embodiments, the compound is of Formula (IIId) or a pharmaceutically acceptable salt thereof.

In some embodiments, the compound of Formula (I) is a compound of Formula (IVa), (IVb), (IVc), or (IVd):

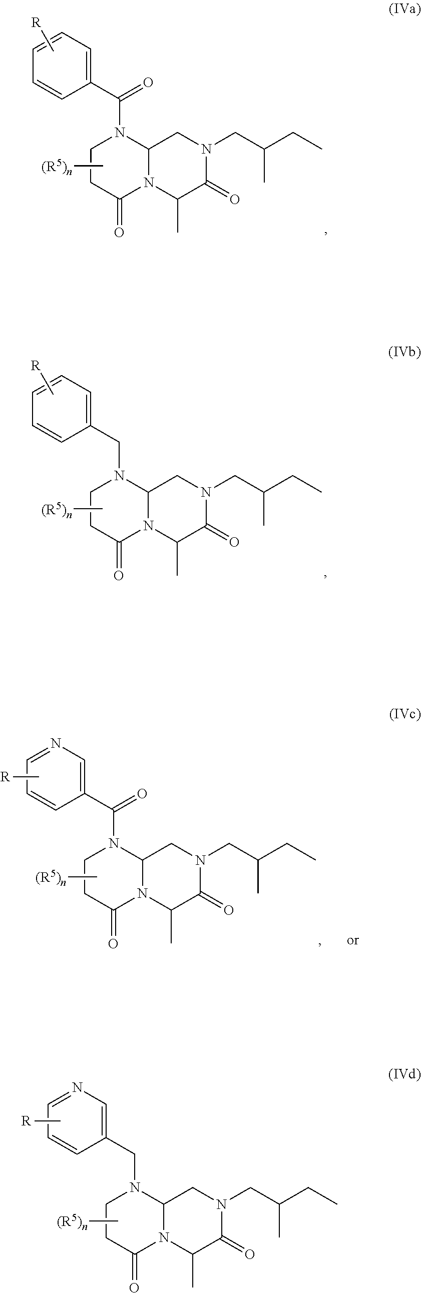

or a pharmaceutically acceptable salt thereof, wherein R⁵ and n are as described for Formula (I), and R represents one or more optional substituents, such as hydroxyl, halo, amino, C₁-C₆ haloalkyl, C₁-C₆ haloalkoxy, as described for Formula (I). In some embodiments, the compound is of Formula (IVa) or a pharmaceutically acceptable salt thereof. In some embodiments, the compound is of Formula (IVb) or a pharmaceutically acceptable salt thereof. In some embodiments, the compound is of Formula (IVc) or a pharmaceutically acceptable salt thereof. In some embodiments, the compound is of Formula (IVd) or a pharmaceutically acceptable salt thereof.

In some embodiments, the compound of Formula (I) is a compound of Formula (V):

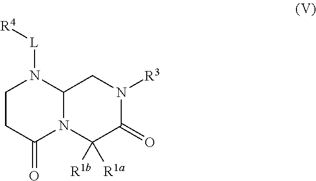

or a pharmaceutically acceptable salt thereof, wherein L, Rid, R^{1b}, R³, and R⁴ are as described for Formula (I). In some embodiments, L is —C(═O)— or —CH₂—; R^{1a} and R^{1b} are independently H or C₁-C₃ alkyl optionally substituted with —CO₂H; R³ is C₄-C₅ alkyl, C₄-C₅ alkenyl, or C₁-C₃ alkyl substituted with C₃-C₅ cycloalkyl; and R⁴ is phenyl or pyridyl substituted with 1-3 substituents selected from —CF₃, —OCHF₂, —OH, fluoro, and chloro. In some variations, one of R^{1a} and R^{1b} is H and the other is C₁-C₃ alkyl, such as methyl.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to L of Formula (I) may be combined with every description, variation, embodiment, or aspect of R¹, R^{1b}, R², R³, R⁴, R⁵, R⁶, R⁷, and n the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II), (IIa), (IIb), (IIc), (IId), (IIe), (IIIa), (IIIb), (IIIc), (IIId), (IVa), (IVb), (IVc), (IVd), and (V), and are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Com- | |
|---|---|
| pound | Structure |
| 1a | 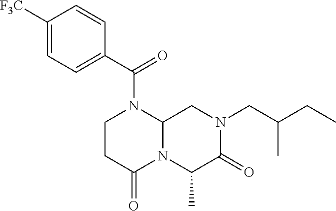 [SMILES: CCC(C)CN1CC2N(C(=O)c3ccc(C(F)(F)F)cc3)CCC(=O)N2[C@@H](C)C1=O] |
| 1b | 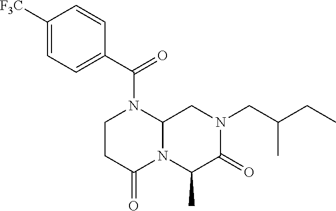 [SMILES: CCC(C)CN1CC2N(C(=O)c3ccc(C(F)(F)F)cc3)CCC(=O)N2[C@H](C)C1=O] |
| 2a | 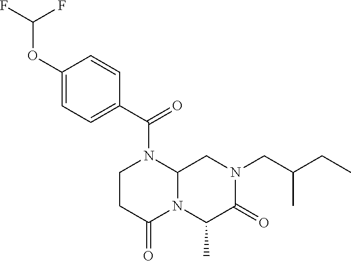 [SMILES: CCC(C)CN1CC2N(C(=O)c3ccc(OC(F)F)cc3)CCC(=O)N2[C@@H](C)C1=O] |
| 2b | 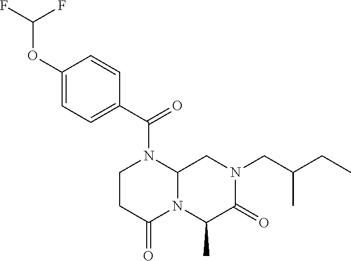 [SMILES: CCC(C)CN1CC2N(C(=O)c3ccc(OC(F)F)cc3)CCC(=O)N2[C@H](C)C1=O] |
| 3a | 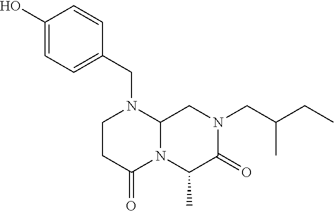 [SMILES: CCC(C)CN1CC2N(Cc3ccc(O)cc3)CCC(=O)N2[C@@H](C)C1=O] |
| 3b | 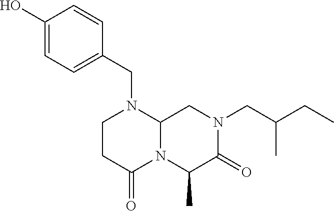 [SMILES: CCC(C)CN1CC2N(Cc3ccc(O)cc3)CCC(=O)N2[C@H](C)C1=O] |
| 4a | 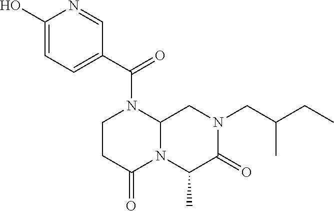 [SMILES: CCC(C)CN1CC2N(C(=O)c3ccc(O)nc3)CCC(=O)N2[C@@H](C)C1=O] |
| 4b | 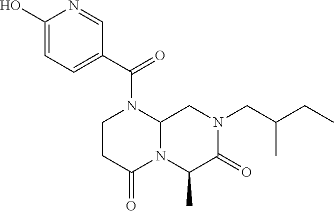 [SMILES: CCC(C)CN1CC2N(C(=O)c3ccc(O)nc3)CCC(=O)N2[C@H](C)C1=O] |
| 5a | 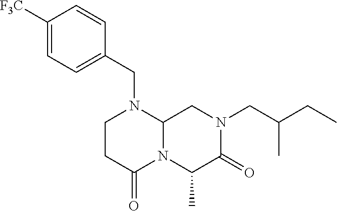 [SMILES: CCC(C)CN1CC2N(Cc3ccc(C(F)(F)F)cc3)CCC(=O)N2[C@@H](C)C1=O] |
| 5b | 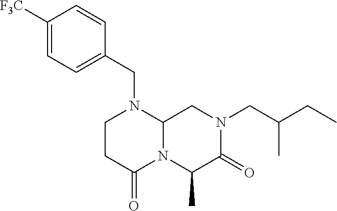 [SMILES: CCC(C)CN1CC2N(Cc3ccc(C(F)(F)F)cc3)CCC(=O)N2[C@H](C)C1=O] |
| 6a | 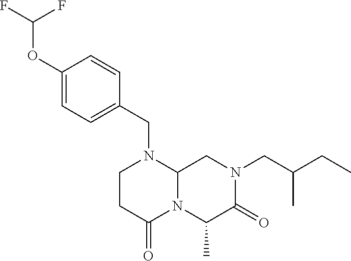 [SMILES: CCC(C)CN1CC2N(Cc3ccc(OC(F)F)cc3)CCC(=O)N2[C@@H](C)C1=O] |
| 6b | 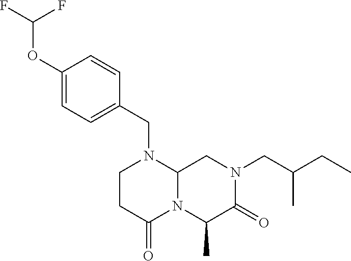 [SMILES: CCC(C)CN1CC2N(Cc3ccc(OC(F)F)cc3)CCC(=O)N2[C@H](C)C1=O] |
| 7a | 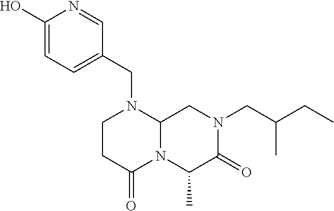 [SMILES: CCC(C)CN1CC2N(Cc3ccc(O)nc3)CCC(=O)N2[C@@H](C)C1=O] |
| 7b | 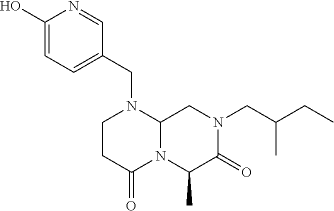 [SMILES: CCC(C)CN1CC2N(Cc3ccc(O)nc3)CCC(=O)N2[C@H](C)C1=O] |
| 8a | 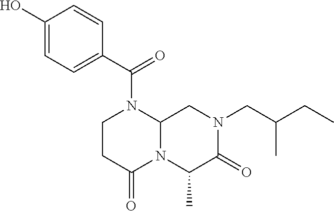 [SMILES: CCC(C)CN1CC2N(C(=O)c3ccc(O)cc3)CCC(=O)N2[C@@H](C)C1=O] |
| 8b | 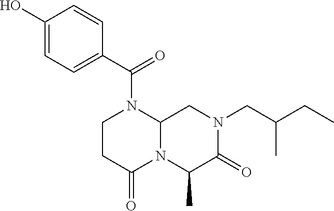 [SMILES: CCC(C)CN1CC2N(C(=O)c3ccc(O)cc3)CCC(=O)N2[C@H](C)C1=O] |
| 9 | 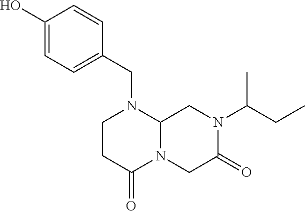 [SMILES: CCC(C)N1CC2N(Cc3ccc(O)cc3)CCC(=O)N2CC1=O] |
| 10 | 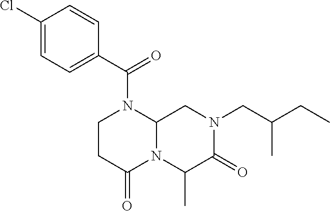 [SMILES: CCC(C)CN1CC2N(C(=O)c3ccc(Cl)cc3)CCC(=O)N2C(C)C1=O] |
| 11 | 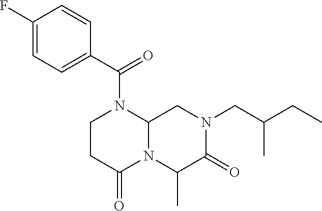 [SMILES: CCC(C)CN1CC2N(C(=O)c3ccc(F)cc3)CCC(=O)N2C(C)C1=O] |
| 12 | 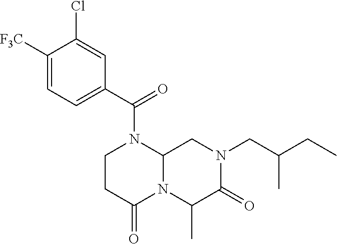 [SMILES: CCC(C)CN1CC2N(C(=O)c3ccc(C(F)(F)F)c(Cl)c3)CCC(=O)N2C(C)C1=O] |
| 13 | 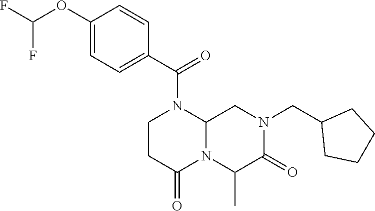 [SMILES: CC1C(=O)N(CC2CCCC2)CC2N(C(=O)c3ccc(OC(F)F)cc3)CCC(=O)N12] |
| 14 | 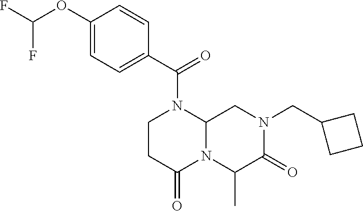 [SMILES: CC1C(=O)N(CC2CCC2)CC2N(C(=O)c3ccc(OC(F)F)cc3)CCC(=O)N12] |
| 15 | 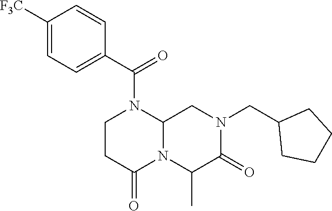 [SMILES: CC1C(=O)N(CC2CCCC2)CC2N(C(=O)c3ccc(C(F)(F)F)cc3)CCC(=O)N12] |
| 16 | 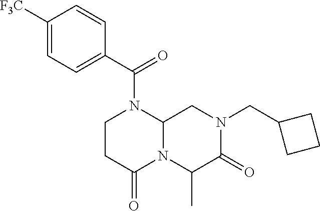 [SMILES: CC1C(=O)N(CC2CCC2)CC2N(C(=O)c3ccc(C(F)(F)F)cc3)CCC(=O)N12] |
| 17 | 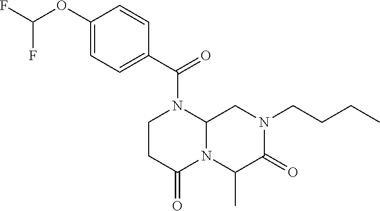 [SMILES: CCCCN1CC2N(C(=O)c3ccc(OC(F)F)cc3)CCC(=O)N2C(C)C1=O] |
| 18 | 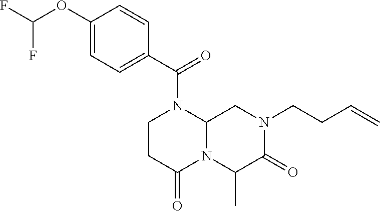 [SMILES: C=CCCN1CC2N(C(=O)c3ccc(OC(F)F)cc3)CCC(=O)N2C(C)C1=O] |
| 19 | 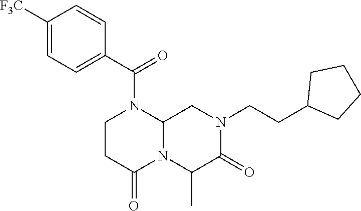 [SMILES: CC1C(=O)N(CCC2CCCC2)CC2N(C(=O)c3ccc(C(F)(F)F)cc3)CCC(=O)N12] |
| 20 | 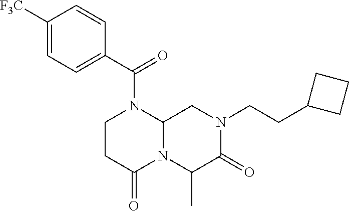 [SMILES: CC1C(=O)N(CCC2CCC2)CC2N(C(=O)c3ccc(C(F)(F)F)cc3)CCC(=O)N12] |
| 21 | 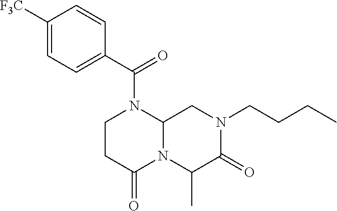 [SMILES: CCCCN1CC2N(C(=O)c3ccc(C(F)(F)F)cc3)CCC(=O)N2C(C)C1=O] |
| 22 | 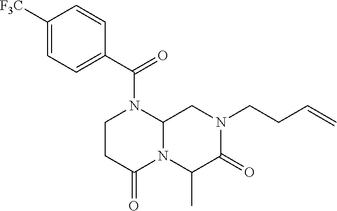 [SMILES: C=CCCN1CC2N(C(=O)c3ccc(C(F)(F)F)cc3)CCC(=O)N2C(C)C1=O] |
| 23 | 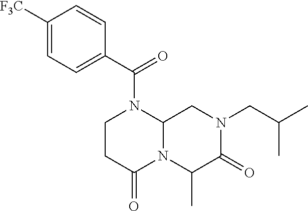 [SMILES: CC(C)CN1CC2N(C(=O)c3ccc(C(F)(F)F)cc3)CCC(=O)N2C(C)C1=O] |
| 24 | 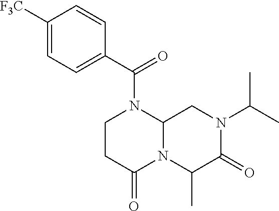 [SMILES: CC(C)N1CC2N(C(=O)c3ccc(C(F)(F)F)cc3)CCC(=O)N2C(C)C1=O] |
| 25 | 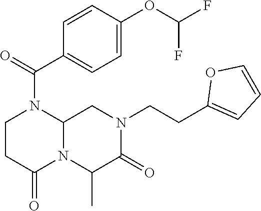 [SMILES: CC1C(=O)N(CCc2ccco2)CC2N(C(=O)c3ccc(OC(F)F)cc3)CCC(=O)N12] |
| 26 | 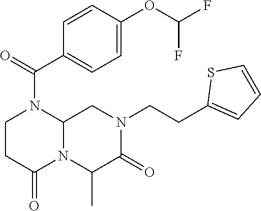 [SMILES: CC1C(=O)N(CCc2cccs2)CC2N(C(=O)c3ccc(OC(F)F)cc3)CCC(=O)N12] |
| 27 | 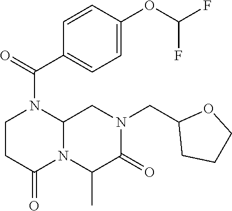 [SMILES: CC1C(=O)N(CC2CCCO2)CC2N(C(=O)c3ccc(OC(F)F)cc3)CCC(=O)N12] |
| 28 | 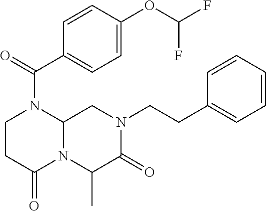 [SMILES: CC1C(=O)N(CCc2ccccc2)CC2N(C(=O)c3ccc(OC(F)F)cc3)CCC(=O)N12] |
| 29 | 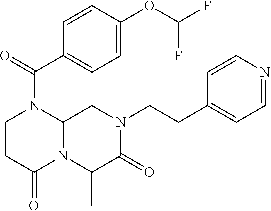 [SMILES: CC1C(=O)N(CCc2ccncc2)CC2N(C(=O)c3ccc(OC(F)F)cc3)CCC(=O)N12] |
| 30 | 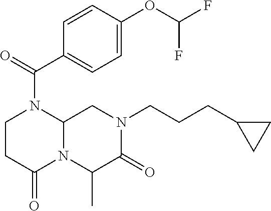 [SMILES: CC1C(=O)N(CCCC2CC2)CC2N(C(=O)c3ccc(OC(F)F)cc3)CCC(=O)N12] |
| 31 | 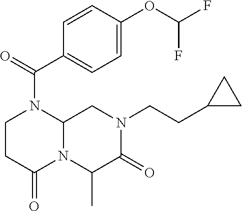 [SMILES: CC1C(=O)N(CCC2CC2)CC2N(C(=O)c3ccc(OC(F)F)cc3)CCC(=O)N12] |
| 32 | 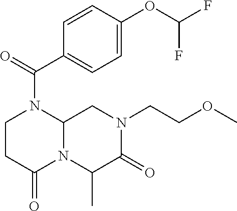 [SMILES: COCCN1CC2N(C(=O)c3ccc(OC(F)F)cc3)CCC(=O)N2C(C)C1=O] |
| 33 | 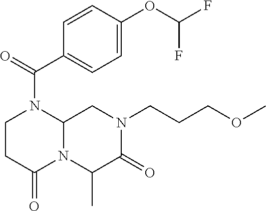 [SMILES: COCCCN1CC2N(C(=O)c3ccc(OC(F)F)cc3)CCC(=O)N2C(C)C1=O] |
| 34 | 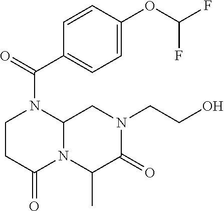 [SMILES: CC1C(=O)N(CCO)CC2N(C(=O)c3ccc(OC(F)F)cc3)CCC(=O)N12] |
| 35 | 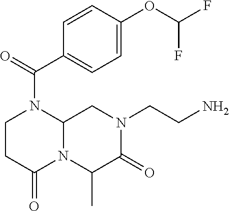 [SMILES: CC1C(=O)N(CCN)CC2N(C(=O)c3ccc(OC(F)F)cc3)CCC(=O)N12] |
| 36 | 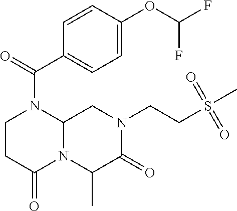 [SMILES: CC1C(=O)N(CCS(C)(=O)=O)CC2N(C(=O)c3ccc(OC(F)F)cc3)CCC(=O)N12] |
| 37 | 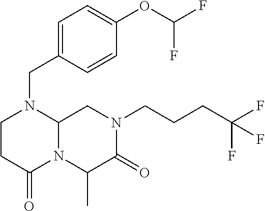 [SMILES: CC1C(=O)N(CCCC(F)(F)F)CC2N(Cc3ccc(OC(F)F)cc3)CCC(=O)N12] |
| 38 | 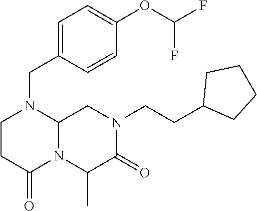 [SMILES: CC1C(=O)N(CCC2CCCC2)CC2N(Cc3ccc(OC(F)F)cc3)CCC(=O)N12] |
| 39 | 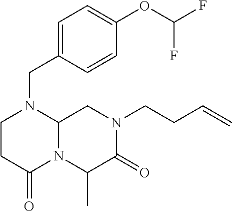 [SMILES: C=CCCN1CC2N(Cc3ccc(OC(F)F)cc3)CCC(=O)N2C(C)C1=O] |
| 40 | 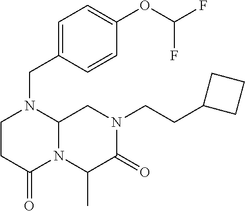 [SMILES: CC1C(=O)N(CCC2CCC2)CC2N(Cc3ccc(OC(F)F)cc3)CCC(=O)N12] |
| 41 | 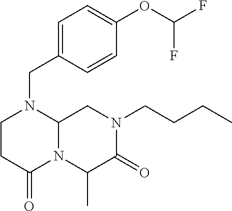 [SMILES: CCCCN1CC2N(Cc3ccc(OC(F)F)cc3)CCC(=O)N2C(C)C1=O] |
| 42 | 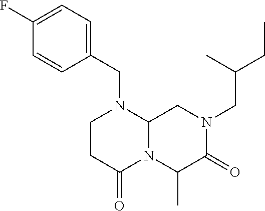 [SMILES: CCC(C)CN1CC2N(Cc3ccc(F)cc3)CCC(=O)N2C(C)C1=O] |
| 43 | 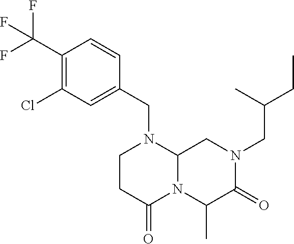 [SMILES: CCC(C)CN1CC2N(Cc3ccc(C(F)(F)F)c(Cl)c3)CCC(=O)N2C(C)C1=O] |
| 44 | 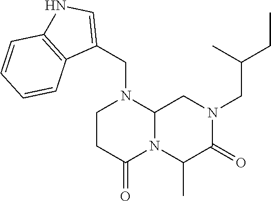 [SMILES: CCC(C)CN1CC2N(Cc3c[nH]c4ccccc34)CCC(=O)N2C(C)C1=O] |
| 45 | 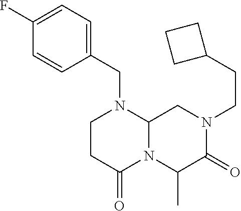 [SMILES: CC1C(=O)N(CCC2CCC2)CC2N(Cc3ccc(F)cc3)CCC(=O)N12] |
| 46 | 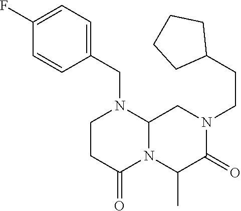 [SMILES: CC1C(=O)N(CCC2CCCC2)CC2N(Cc3ccc(F)cc3)CCC(=O)N12] |
| 47 | 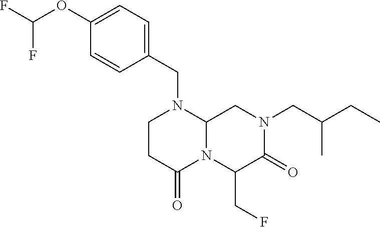 [SMILES: CCC(C)CN1CC2N(Cc3ccc(OC(F)F)cc3)CCC(=O)N2C(CF)C1=O] |
| 48 | 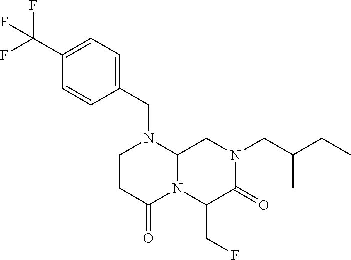 [SMILES: CCC(C)CN1CC2N(Cc3ccc(C(F)(F)F)cc3)CCC(=O)N2C(CF)C1=O] |
| 49 | 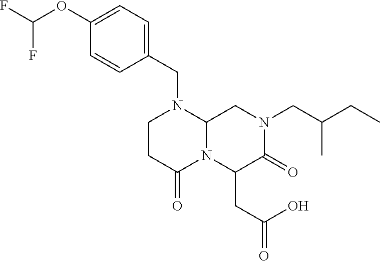 [SMILES: CCC(C)CN1CC2N(Cc3ccc(OC(F)F)cc3)CCC(=O)N2C(CC(=O)O)C1=O] |
| 50 | 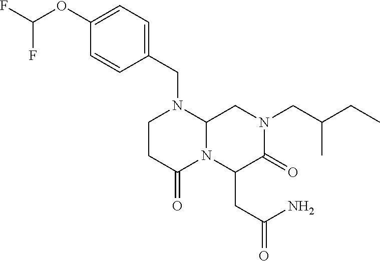 [SMILES: CCC(C)CN1CC2N(Cc3ccc(OC(F)F)cc3)CCC(=O)N2C(CC(N)=O)C1=O] |
| 51 | 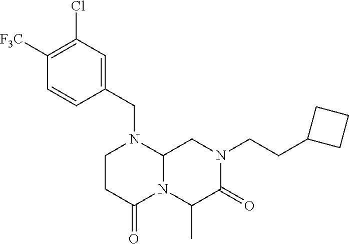 [SMILES: CC1C(=O)N(CCC2CCC2)CC2N(Cc3ccc(C(F)(F)F)c(Cl)c3)CCC(=O)N12] |
| 52 | 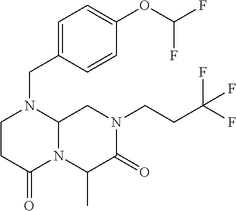 [SMILES: CC1C(=O)N(CCC(F)(F)F)CC2N(Cc3ccc(OC(F)F)cc3)CCC(=O)N12] |
| 53 | 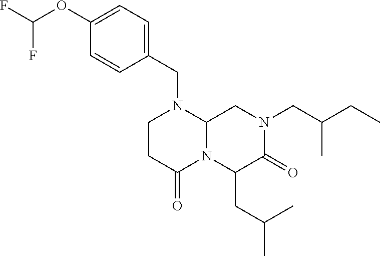 [SMILES: CCC(C)CN1CC2N(Cc3ccc(OC(F)F)cc3)CCC(=O)N2C(CC(C)C)C1=O] |
| 54 | 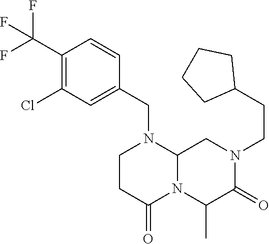 [SMILES: CC1C(=O)N(CCC2CCCC2)CC2N(Cc3ccc(C(F)(F)F)c(Cl)c3)CCC(=O)N12] |
| 55 | 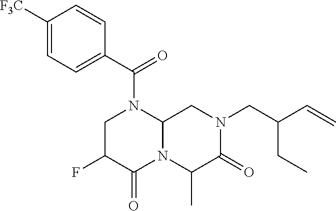 [SMILES: C=CC(CC)CN1CC2N(C(=O)c3ccc(C(F)(F)F)cc3)CC(F)C(=O)N2C(C)C1=O] |
| 56 | 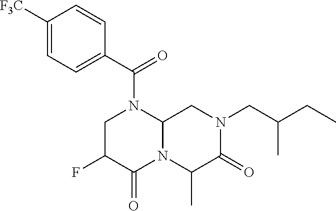 [SMILES: CCC(C)CN1CC2N(C(=O)c3ccc(C(F)(F)F)cc3)CC(F)C(=O)N2C(C)C1=O] |
| 57 | 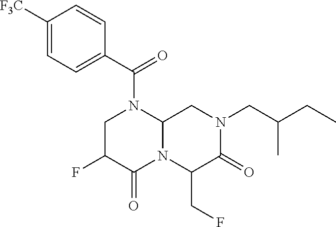 [SMILES: CCC(C)CN1CC2N(C(=O)c3ccc(C(F)(F)F)cc3)CC(F)C(=O)N2C(CF)C1=O] |
| 58 | 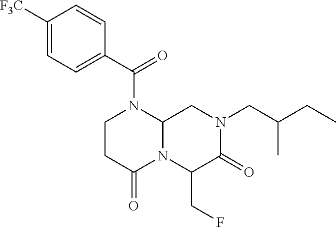 [SMILES: CCC(C)CN1CC2N(C(=O)c3ccc(C(F)(F)F)cc3)CCC(=O)N2C(CF)C1=O] |
| 59 | 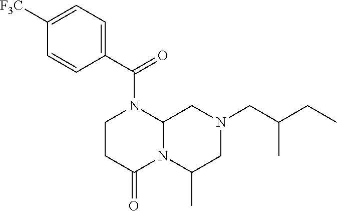 [SMILES: CCC(C)CN1CC(C)N2C(=O)CCN(C(=O)c3ccc(C(F)(F)F)cc3)C2C1] |
| 60 | 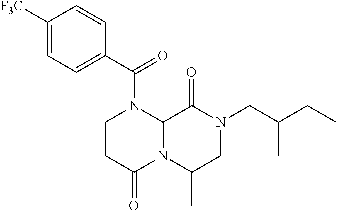 [SMILES: CCC(C)CN1CC(C)N2C(=O)CCN(C(=O)c3ccc(C(F)(F)F)cc3)C2C1=O] |
| 61 | 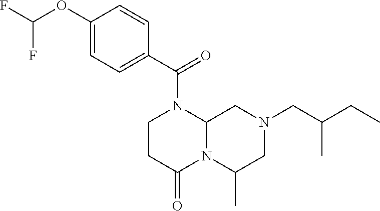 [SMILES: CCC(C)CN1CC(C)N2C(=O)CCN(C(=O)c3ccc(OC(F)F)cc3)C2C1] |
| 62 | 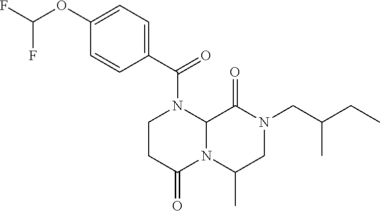 [SMILES: CCC(C)CN1CC(C)N2C(=O)CCN(C(=O)c3ccc(OC(F)F)cc3)C2C1=O] |
| 63 | 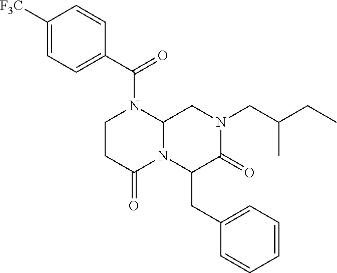 [SMILES: CCC(C)CN1CC2N(C(=O)c3ccc(C(F)(F)F)cc3)CCC(=O)N2C(Cc2ccccc2)C1=O] |
| 64 | 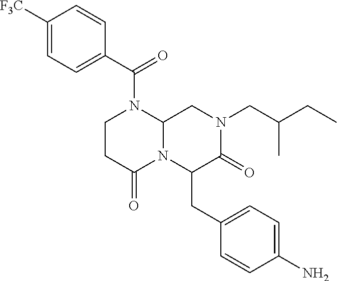 [SMILES: CCC(C)CN1CC2N(C(=O)c3ccc(C(F)(F)F)cc3)CCC(=O)N2C(Cc2ccc(N)cc2)C1=O] |
| 65 | 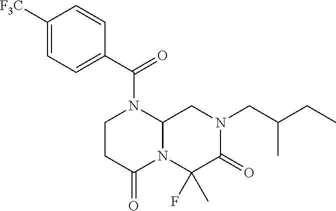 [SMILES: CCC(C)CN1CC2N(C(=O)c3ccc(C(F)(F)F)cc3)CCC(=O)N2C(C)(F)C1=O] |
| 66 | 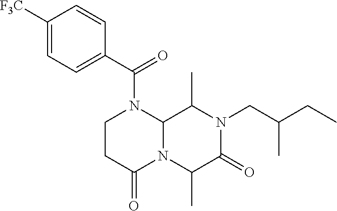 [SMILES: CCC(C)CN1C(=O)C(C)N2C(=O)CCN(C(=O)c3ccc(C(F)(F)F)cc3)C2C1C] |
| 67 | 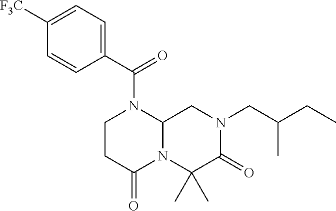 [SMILES: CCC(C)CN1CC2N(C(=O)c3ccc(C(F)(F)F)cc3)CCC(=O)N2C(C)(C)C1=O] |
| 68 | 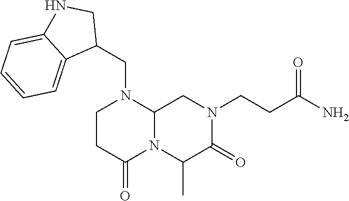 [SMILES: CC1C(=O)N(CCC(N)=O)CC2N(CC3CNc4ccccc43)CCC(=O)N12] |
| 69 | 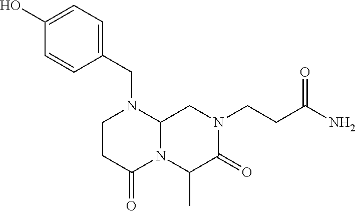 [SMILES: CC1C(=O)N(CCC(N)=O)CC2N(Cc3ccc(O)cc3)CCC(=O)N12] |
| 70 | 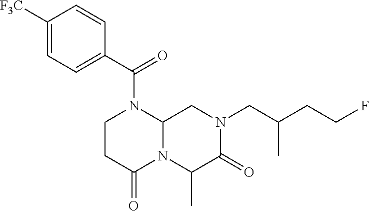 [SMILES: CC(CCF)CN1CC2N(C(=O)c3ccc(C(F)(F)F)cc3)CCC(=O)N2C(C)C1=O] |
| 71 | 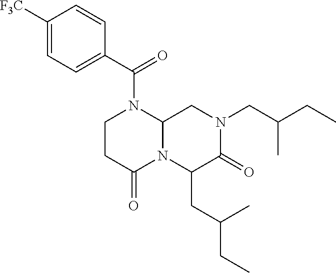 [SMILES: CCC(C)CC1C(=O)N(CC(C)CC)CC2N(C(=O)c3ccc(C(F)(F)F)cc3)CCC(=O)N12] |
| 72 | 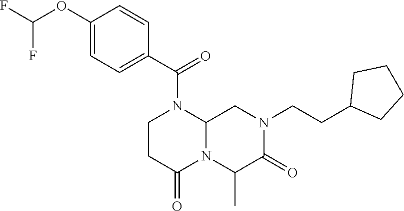 [SMILES: CC1C(=O)N(CCC2CCCC2)CC2N(C(=O)c3ccc(OC(F)F)cc3)CCC(=O)N12] |
| 73 | 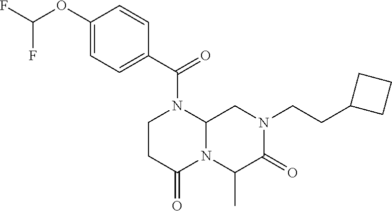 [SMILES: CC1C(=O)N(CCC2CCC2)CC2N(C(=O)c3ccc(OC(F)F)cc3)CCC(=O)N12] |
| 74 | 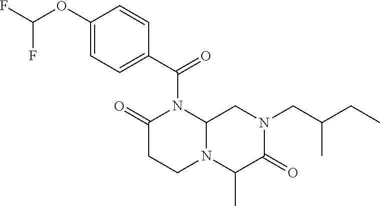 [SMILES: CCC(C)CN1CC2N(C(=O)c3ccc(OC(F)F)cc3)C(=O)CCN2C(C)C1=O] |

or a pharmaceutically acceptable salt thereof.

In some embodiments, the compound of Formula (I) is not Compound 3a, 3b, 9, 10, 13, 15, 16, 18, 21, 23-29, 31-41, 43-48, 50, 52, or 54.

In some embodiments, provided is a compound selected from the compounds in Table 1A or a pharmaceutically acceptable salt thereof. Although certain compounds described in the present disclosure, including in Table 1A, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1A, are herein described.

TABLE 1A

| Com- | |
|---|---|
| pound | Structure |
| 1a | 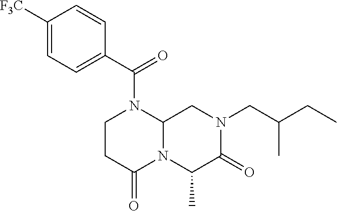 [SMILES: CCC(C)CN1CC2N(C(=O)c3ccc(C(F)(F)F)cc3)CCC(=O)N2[C@@H](C)C1=O] |
| 1b | 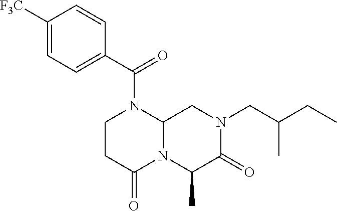 [SMILES: CCC(C)CN1CC2N(C(=O)c3ccc(C(F)(F)F)cc3)CCC(=O)N2[C@H](C)C1=O] |
| 2a | 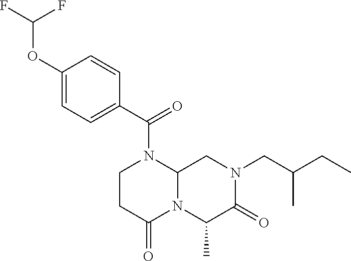 [SMILES: CCC(C)CN1CC2N(C(=O)c3ccc(OC(F)F)cc3)CCC(=O)N2[C@@H](C)C1=O] |
| 2b | 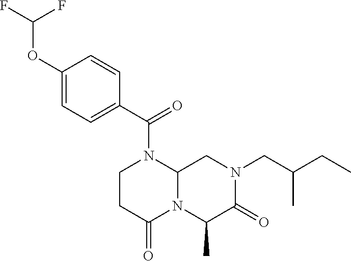 [SMILES: CCC(C)CN1CC2N(C(=O)c3ccc(OC(F)F)cc3)CCC(=O)N2[C@H](C)C1=O] |
| 4a | 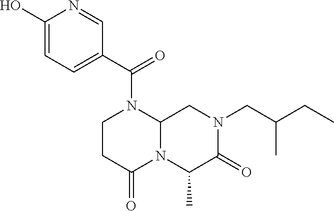 [SMILES: CCC(C)CN1CC2N(C(=O)c3ccc(O)nc3)CCC(=O)N2[C@@H](C)C1=O] |
| 4b | 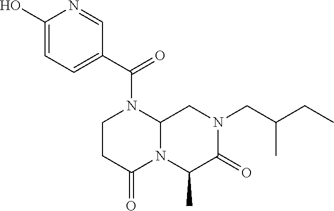 [SMILES: CCC(C)CN1CC2N(C(=O)c3ccc(O)nc3)CCC(=O)N2[C@H](C)C1=O] |
| 5a | 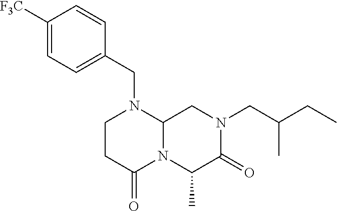 [SMILES: CCC(C)CN1CC2N(Cc3ccc(C(F)(F)F)cc3)CCC(=O)N2[C@@H](C)C1=O] |
| 5b | 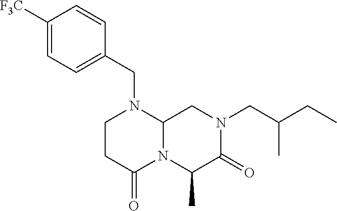 [SMILES: CCC(C)CN1CC2N(Cc3ccc(C(F)(F)F)cc3)CCC(=O)N2[C@H](C)C1=O] |
| 6a | 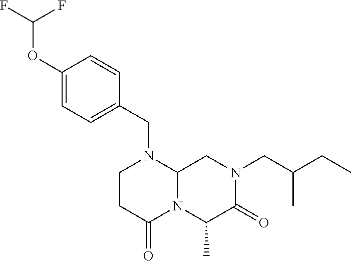 [SMILES: CCC(C)CN1CC2N(Cc3ccc(OC(F)F)cc3)CCC(=O)N2[C@@H](C)C1=O] |
| 6b | 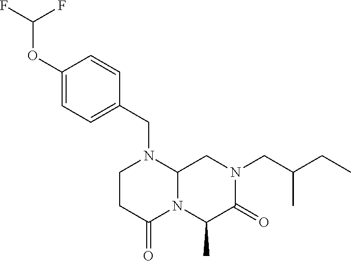 [SMILES: CCC(C)CN1CC2N(Cc3ccc(OC(F)F)cc3)CCC(=O)N2[C@H](C)C1=O] |
| 7a | 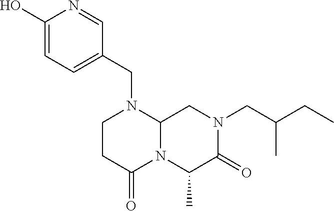 [SMILES: CCC(C)CN1CC2N(Cc3ccc(O)nc3)CCC(=O)N2[C@@H](C)C1=O] |
| 7b | 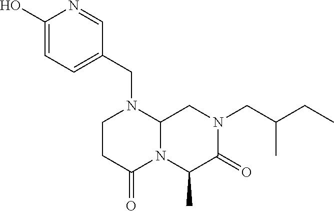 [SMILES: CCC(C)CN1CC2N(Cc3ccc(O)nc3)CCC(=O)N2[C@H](C)C1=O] |
| 8a | 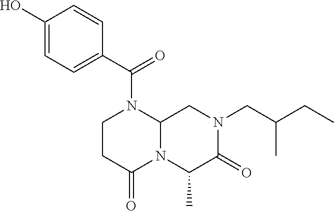 [SMILES: CCC(C)CN1CC2N(C(=O)c3ccc(O)cc3)CCC(=O)N2[C@@H](C)C1=O] |
| 8b | 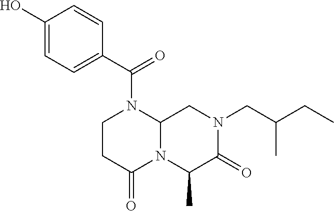 [SMILES: CCC(C)CN1CC2N(C(=O)c3ccc(O)cc3)CCC(=O)N2[C@H](C)C1=O] |
| 11 | 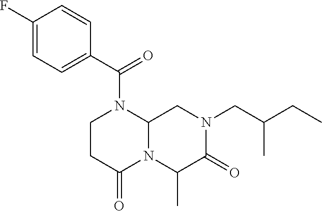 [SMILES: CCC(C)CN1CC2N(C(=O)c3ccc(F)cc3)CCC(=O)N2C(C)C1=O] |
| 12 | 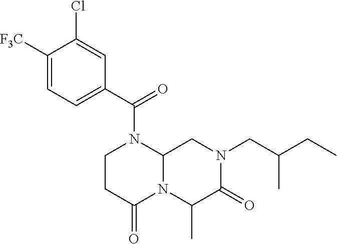 [SMILES: CCC(C)CN1CC2N(C(=O)c3ccc(C(F)(F)F)c(Cl)c3)CCC(=O)N2C(C)C1=O] |
| 14 | 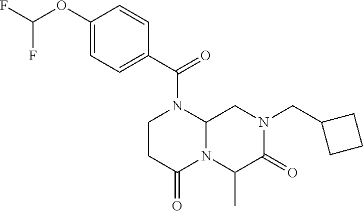 [SMILES: CC1C(=O)N(CC2CCC2)CC2N(C(=O)c3ccc(OC(F)F)cc3)CCC(=O)N12] |
| 17 | 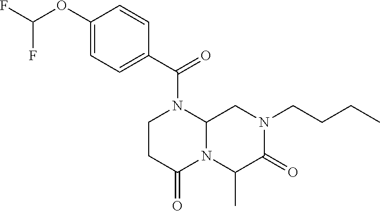 [SMILES: CCCCN1CC2N(C(=O)c3ccc(OC(F)F)cc3)CCC(=O)N2C(C)C1=O] |
| 19 | 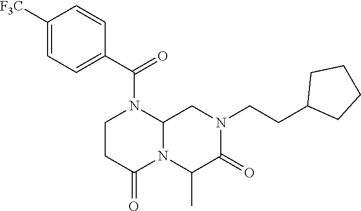 [SMILES: CC1C(=O)N(CCC2CCCC2)CC2N(C(=O)c3ccc(C(F)(F)F)cc3)CCC(=O)N12] |
| 20 | 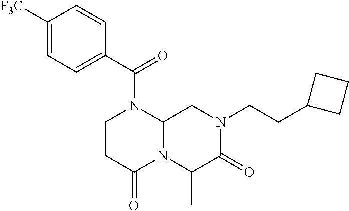 [SMILES: CC1C(=O)N(CCC2CCC2)CC2N(C(=O)c3ccc(C(F)(F)F)cc3)CCC(=O)N12] |
| 22 | 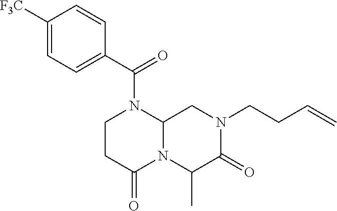 [SMILES: C=CCCN1CC2N(C(=O)c3ccc(C(F)(F)F)cc3)CCC(=O)N2C(C)C1=O] |
| 30 | 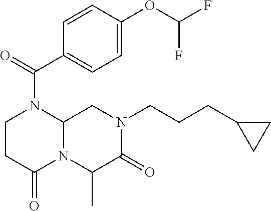 [SMILES: CC1C(=O)N(CCCC2CC2)CC2N(C(=O)c3ccc(OC(F)F)cc3)CCC(=O)N12] |
| 42 | 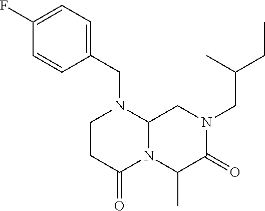 [SMILES: CCC(C)CN1CC2N(Cc3ccc(F)cc3)CCC(=O)N2C(C)C1=O] |
| 49 | 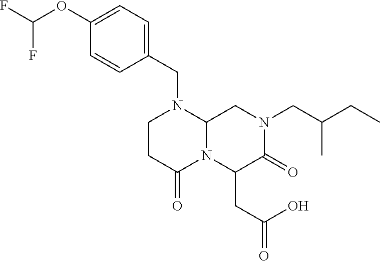 [SMILES: CCC(C)CN1CC2N(Cc3ccc(OC(F)F)cc3)CCC(=O)N2C(CC(=O)O)C1=O] |
| 51 | 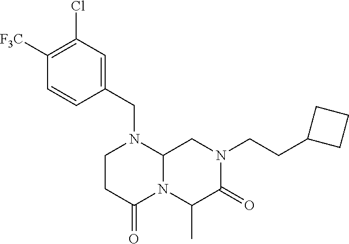 [SMILES: CC1C(=O)N(CCC2CCC2)CC2N(Cc3ccc(C(F)(F)F)c(Cl)c3)CCC(=O)N12] |
| 53 | 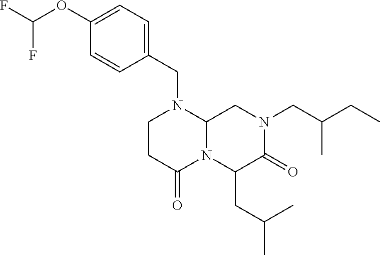 [SMILES: CCC(C)CN1CC2N(Cc3ccc(OC(F)F)cc3)CCC(=O)N2C(CC(C)C)C1=O] |

or a pharmaceutically acceptable salt thereof.

It is understood that in the present description, combinations of substituents and/or variables of the depicted formulae are permissible only if such contributions result in stable compounds.

Furthermore, all compounds of Formula (I) which exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

Compounds of Formula (I), or a pharmaceutically acceptable salt, isotopic form, or stereoisomer thereof, can be prepared by using organic chemistry synthesis methods known in the art. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described herein.

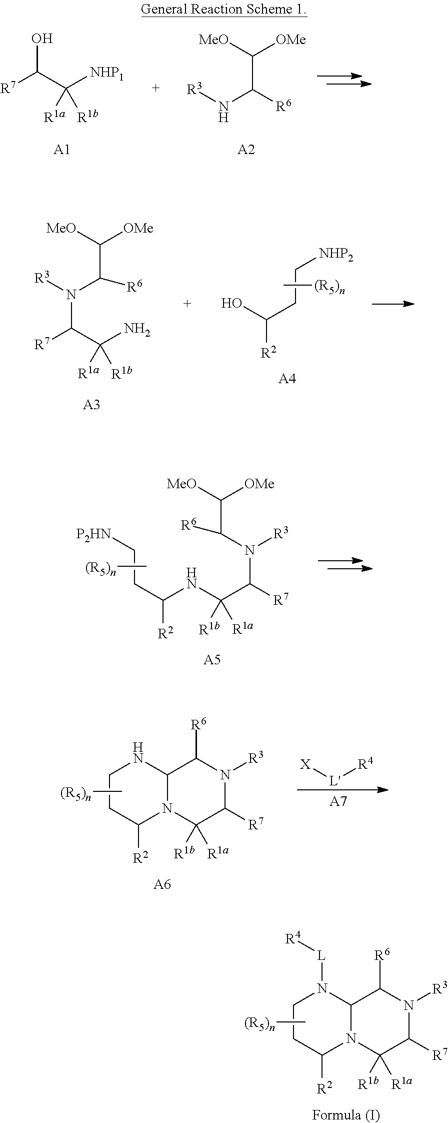

General Reaction Scheme 1 provides an exemplary method for preparation of compounds of Formula (I). R^{1a}, R^{1b}, R², R³, R⁴, R⁵, R⁶, R⁷, L, and n in General Reaction Scheme 1 are as defined herein. X is a reactive moiety selected to facilitate the desired reaction (e.g., halo). P₁ and P₂ are suitable protecting groups. L′ is selected such that a desired L moiety results from the reaction between L′-R⁴ and the secondary amine. Compounds of structure A1 are purchased or prepared according to methods known in the art. Reaction of A1 with A2 under appropriate coupling conditions (e.g., T₃P and base) yields the product of the coupling reaction between A1 and A2, A3. A3 is then reacted with A4 under suitable coupling conditions (e.g., T₃P and base) to afford compound A5. Compound A5 is then cyclized (e.g., using formic acid) and deprotected (e.g., using piperidine) to afford compound A6. Compound A6 is then reacted with compound A7 to afford the final compound of Formula (I) as shown.

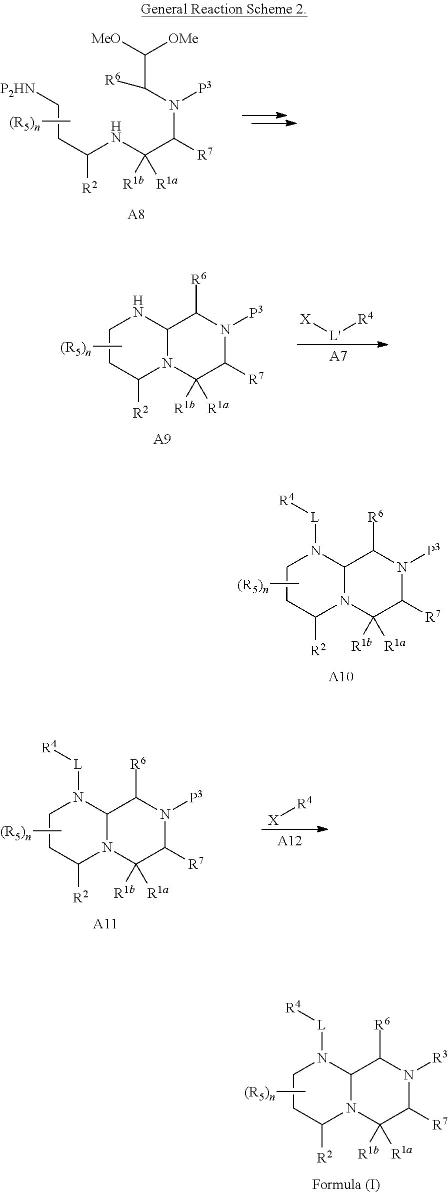

An alternative method for the synthesis of compounds of Formula (I) is depicted in General Reaction Scheme 2. R^{1a}, R^{1b}, R², R³, R⁴, R⁵, R⁶, R⁷, L, and n in General Reaction Scheme 2 are as defined herein. P₂ is a suitable protecting group. Each X is a reactive moiety selected to facilitate the desired reaction (e.g., halo). L′ is selected such that a desired L moiety results from the reaction between L′-R⁴ and the secondary amine. Intermediate A5 is prepared with a removable protecting group P³ (e.g. para-methoxybenzyl) as the R³ group giving intermediate A8. A8 is then cyclized (e.g., using formic acid) and deprotected (e.g., using piperidine) to afford compound A9. Compound A9 is then reacted with A7 to give compound A10. Compound A10 is then deprotected (e.g., with cerica ammonium nitrate) to give compound A11. Compound A11 is then reacted with A12 to provide the final compound of Formula (I).

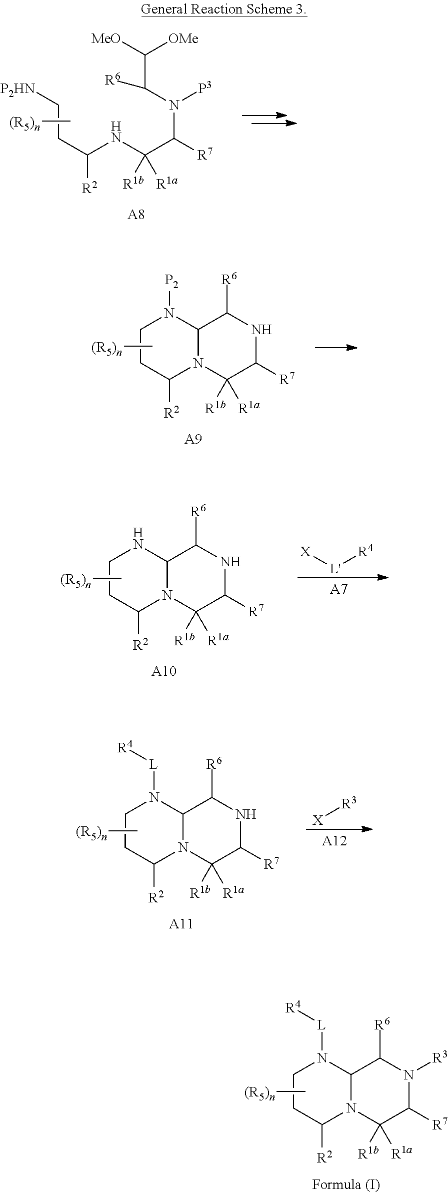

A related method to the one shown in General Reaction Scheme 2 is depicted in General Reaction Scheme 3. In this method, the two amine nitrogen atoms of the bicyclic core are deprotected to provide compound A10, then reacted with A7 to afford compound A11. Subsequent reaction with A12 provides the final compound of Formula (I).

It should be noted that various alternative strategies for preparation of compounds of Formula (I) are available to those of ordinary skill in the art. For example, other compounds of Formula (I) can be prepared according to analogous methods using the appropriate starting material.

It will also be appreciated by those skilled in the art that in the processes for preparing the compounds described herein the functional groups of intermediate compounds may need to be protected by suitable protecting groups. Such functional groups may include hydroxy, amino, and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino and amidino include t-butoxycarbonyl, benzyloxycarbonyl, and the like. Suitable protecting groups for carboxylic acid include alkyl, aryl, or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### Methods of Treatment Uses

Provided herein are methods of treating SARS-CoV-2 infection, also known as long COVID.

In some embodiments, a method of treating long COVID is provided, comprising administering to a patient in need thereof a therapeutically effective amount of a HGF/MET agonist.

In some embodiments, a method of treating long-term sequelae of infection with SARS-CoV-2 is provided, comprising administering to a patient in need thereof a therapeutically effective amount of a HGF/MET positive modulator.

In some embodiments, a method of treating post-acute COVID-19 syndrome is provided, comprising administering to a patient in need thereof a therapeutically effective amount of a HGF/MET positive modulator.

In some embodiments, a method of treating post-COVID-19 condition is provided, comprising administering to a patient in need thereof a therapeutically effective amount of a HGF/MET positive modulator.

In some embodiments, a method of improving event related potential (ERP) P300 latency in a patient diagnosed with long COVID is provided, comprising administering to a patient in need thereof a therapeutically effective amount of a HGF/MET positive modulator.

In some embodiments, a method of slowing the decline in cognition or improving cognition in a patient diagnosed with long COVID is provided, comprising administering to a patient in need thereof a therapeutically effective amount of a HGF/MET positive modulator.

In some embodiments, a method of slowing the decline in the ability to perform activities of daily living and verbal fluency or improving the ability to perform activities of daily living and verbal fluency in a patient diagnosed with long COVID is provided, comprising administering to a patient in need thereof a therapeutically effective amount of a HGF/MET positive modulator.

In some embodiments, a method of slowing the decline in functional or cognitive capacity in a patient diagnosed with long COVID is provided, comprising administering to a patient in need thereof a therapeutically effective amount of a HGF/MET positive modulator.

In some embodiments, a method of slowing clinical decline in a patient diagnosed with long COVID is provided, comprising administering to a patient in need thereof a therapeutically effective amount of a HGF/MET positive modulator.

In some embodiments, a method of improving executive memory function in a patient diagnosed with long COVID is provided, comprising administering to a patient in need thereof a therapeutically effective amount of a HGF/MET positive modulator.

In some embodiments, the HGF/MET positive modulator is an antibody, an aptamer, a peptide, and/or a small molecule.

In some embodiments, the HGF/MET positive modulator is N-hexanoic-Tyr-Ile-(6)aminohexanoic amide (Dihexa) or a pharmaceutically acceptable salt or a prodrug thereof.

In some embodiments, the HGF/MET positive modulator is

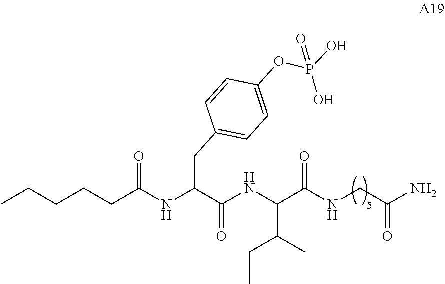

or a pharmaceutically acceptable salt thereof.

In some embodiments, the method comprises administering to the patient 2-90 mg per day of the HGF/MET positive modulator.

In some embodiments, the HGF/MET positive modulator is

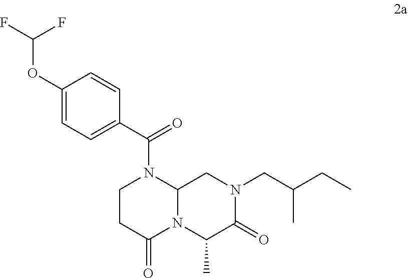 [SMILES: CCC(C)CN1CC2N(C(=O)c3ccc(OC(F)F)cc3)CCC(=O)N2[C@@H](C)C1=O]

or a pharmaceutically acceptable salt thereof.

In some embodiments, the HGF/MET positive modulator is

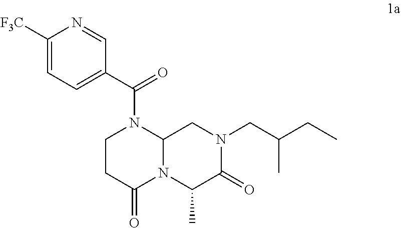 [SMILES: CCC(C)CN1CC2N(C(=O)c3ccc(C(F)(F)F)nc3)CCC(=O)N2[C@@H](C)C1=O]

or a pharmaceutically acceptable salt thereof.

In some embodiments, the HGF/MET positive modulator is a compound of Formula (I):

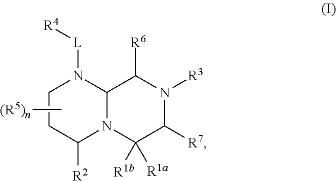

or a pharmaceutically acceptable salt thereof, wherein:
L is a direct bond, —C(═O)—, —(CR^{a}R^{b})ₘ—C(═O)—, —C(═O)—(CR^{a}R^{b})ₘ—, or —(CR^{a}R^{b})ₘ—;
each R^{a} and R^{b} is independently H, C₁-C₆ alkyl, C₂-C₆ alkenyl, or C₂-C₆ alkynyl;
R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl;
R² is H, oxo, or thioxo;
R³ is C₂-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl,
   wherein the 5- to 10-membered heteroarylalkyl or 5- to 10-membered heterocyclylalkyl contains 1-3 heteroatoms selected from nitrogen and oxygen;
R⁴ is C₆-C₁₀ aryl, 5- to 10-membered heteroaryl, or 5- to 10-membered heterocyclyl,
   wherein the 5- to 10-membered heteroaryl or 5- to 10-membered heterocyclyl contains 1-3 heteroatoms selected from nitrogen and oxygen;
each R⁵ is independently C₁-C₆ alkyl, oxo, or halo;
R⁶ is H, C₁-C₆ alkyl, or oxo;
R⁷ is H or oxo;
m is 1 or 2; and
n is an integer from 0 to 3;
wherein each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.

In some embodiments, the Compound of Formula (I) is a of Formula (II), (IIa), (IIb), (IIc), (IId), or (IIe):

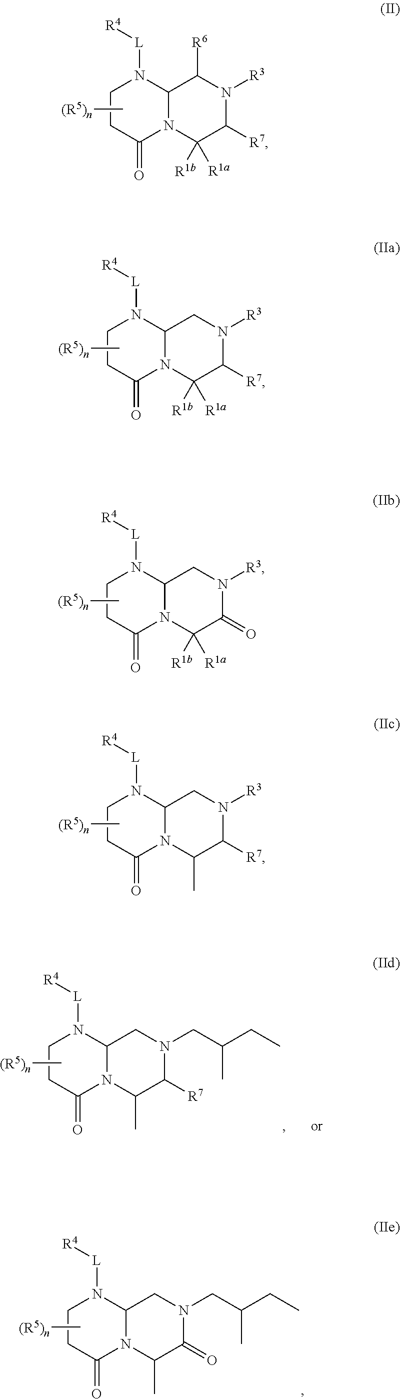

or a pharmaceutically acceptable salt thereof. In some embodiments, the compound is a compound as set forth in Table 1, or a pharmaceutically acceptable salt thereof. In some embodiments, the compound is a compound as set forth in Table 1A, or a pharmaceutically acceptable salt thereof.

In some embodiments, prior to treatment, the patient exhibits one or more symptoms selected from anxiety, apathy, breathlessness, chest pain, cognitive impairment, depression, fatigue, impaired lung function, reduced liver function, increased rate of venous thromboembolism, muscle weakness, idiopathic pulmonary fibrosis, and/or respiratory complications.

In some embodiments, prior to treatment, the patient exhibits cognitive impairment, such as an attention, working memory, processing speed, executive functioning, phonemic fluency, category fluency, memory encoding, memory recall, and/or memory recognition cognitive impairment. In some embodiments, the cognitive impairment suggests an executive pattern or a dysexecutive syndrome.

In some embodiments, the patient has previously been diagnosed with airways disease, asthma such as moderate to severe asthma, bronchiectasis, bronchopulmonary dysplasia, cancer, chronic lung disease, chronic obstructive pulmonary disease (COPD), diabetes, hypertension, interstitial lung disease, pulmonary embolism, and/or pulmonary hypertension.

In some embodiments, the treatment slows the decline in functional or cognitive capacity in the patient. In some embodiments, the treatment improves cognitive capacity in the patient.

In some embodiments, the treatment slows the decline in cognition in the patient. In some embodiments, the treatment improves cognition in the patient.

In some embodiments, the treatment slows the decline in the ability to perform instrumental activities of daily living, basic activities of daily living, financial capacity, and/or verbal fluency in the patient.

In some embodiments, the treatment improves the ability to perform activities of daily living, financial capacity, and/or verbal fluency in the patient.

In some embodiments, the slowing of the decline or the improvement is determined after administering the treatment for at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks.

Various methods are useful in assessing the cognitive capacity of the patient.

In some embodiments, cognitive capacity is assessed by determining the patient's score before and after administering the treatment.

In some embodiments, the patient is assessed by an Instrumental (23-item) Activities of Daily Living (ADL) score. In some embodiments, an Instrumental (23-item) ADL score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves an Instrumental (23-item) ADL score.

In some embodiments, the patient is assessed by a Basic (19-item) Activities of Daily Living (ADL) score. In some embodiments, a Basic (19-item) ADL score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a Basic (19-item) ADL score.

In some embodiments, the patient is assessed by a Financial Capacity Instrument (FCI) score. In some embodiments, an FCI score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves an FCI score.

In some embodiments, the patient has a Mini-Mental State Examination (MMSE) score of less than or equal to 27 prior to the start of treatment. In some embodiments, the method of treatment reduces the rate of decline, stabilizes, or improves a MMSE score.

In some embodiments, the patient is assessed by an Alzheimer's Disease (AD) Assessment Scale-Cognitive Subscale (ADAS-Cog) score. In some embodiments, an ADAS-Cog score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves an ADAS-Cog score.

In some embodiments, the patient is assessed by a Montreal Cognitive Assessment test (MoCA) score. In some embodiments, a MoCA score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a MoCA score.

In some embodiments, the patient is assessed by a Digit Symbol Substitution Test (DSST) score. In some embodiments, a DSST score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a DSST score.

In some embodiments, the patient is assessed by a Disability Assessment for Dementia (DAD) score. In some embodiments, a DAD score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a DAD score.

In some embodiments, the patient is assessed by a neuropsychiatric inventory (NPI) score. In some embodiments, an NPI score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves an NPI score.

In some embodiments, the patient is assessed by a Controlled Oral Word Association Test (COWAT) score. In some embodiments, a COWAT score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a COWAT score.

In some embodiments, the patient is assessed by a Number Span forward and/or backward score. In some embodiments, a Number Span forward and/or backward score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a Number Span forward and/or backward score.

In some embodiments, the patient is assessed by a Trail Making Test Part A and/or Part B score. In some embodiments, a Trail Making Test Part A and/or Part B score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a Trail Making Test Part A and/or Part B score.

In some embodiments, the patient is assessed by a phonemic and category fluency score. In some embodiments, a phonemic and category fluency score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a phonemic and category fluency score.

In some embodiments, the patient is assessed by a Hopkins Verbal Learning Test-Revised score. In some embodiments, a Hopkins Verbal Learning Test-Revised score is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment. In some embodiments, the method of treatment reduces the rate of decline, stabilizes, or improves a Hopkins Verbal Learning Test-Revised score.

In some embodiments, the treatment provides fast improvement or normalization of P300 amplitude values and/or fast improvement or normalization of event-related potential (ERP) P300 latency values. In some embodiments, the treatment provides fast improvement or normalization of ERP P300 latency values with some maintenance of effect at 4 weeks after discontinuation of treatment. In some embodiments, the treatment provides fast improvement or normalization of ERP P300 latency values, which is maintained at 4 weeks after discontinuation of treatment. In some embodiments, the treatment improves event-related potential (ERP) P300 latency. In some embodiments, the treatment provides improvement or normalization of P300 values by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

In some embodiments, the treatment comprises administering the compound of formula A19 or the pharmaceutically acceptable salt thereof, such as a sodium salt of the compound of formula A19, such as a monosodium salt of the compound of formula A19, such as ATH-1017, by subcutaneous injection.

In some embodiments, the treatment comprises administering the compound of formula A19 or the pharmaceutically acceptable salt thereof, such as a sodium salt of the compound of formula A19, such as a monosodium salt of the compound of formula A19, such as ATH-1017, for 26 weeks or more.

In some embodiments, the treatment comprises administering the compound of formula 2a or the pharmaceutically acceptable salt thereof, by subcutaneous injection or by oral dosage, such as by solid oral dosage form.

In some embodiments, the treatment comprises administering the compound of formula 1a or the pharmaceutically acceptable salt thereof, by subcutaneous injection or by oral dosage, such as by solid oral dosage form.

In some embodiments, the treatment comprises administering the compound of formula 2a or the pharmaceutically acceptable salt thereof, for 26 weeks or more.

In some embodiments, the treatment comprises administering the compound of formula 1a or the pharmaceutically acceptable salt thereof, for 26 weeks or more.

In some embodiments, the method of treatment has an acceptable safety and tolerability profile. In some embodiments, the method of treatment is generally safe and well tolerated.

### Pharmaceutical Compositions

In some embodiments, the method includes administering the HGF/MET agonist by subcutaneous injection.

In some embodiments, the method includes administering ATH-1017 by subcutaneous injection.

Pharmaceutical compositions for the drugs provided herein may be in a form suitable for the administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, PA).

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as, for example, oleic acid may likewise be used in the preparation of injectables.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The amount of active ingredient that may be combined with the carrier material to produce a single dosage form will vary depending upon the host treated and the particular mode of administration, such as subcutaneous injection. The pharmaceutical composition can be prepared to provide easily measurable amounts for administration. For example, in some embodiments, ATH-1017 is formulated for subcutaneous administration, provided in a pre-filled syringe containing 1 mL of 40 mg/mL ATH-1017 or 70 mg/mL ATH-1017. In some embodiments, the ATH-1017 is in a solution comprising 10 mM sodium phosphate.

In some embodiments, the method includes administering the HGF/MET positive modulator by oral dosage form.

In some embodiments, the method includes administering Compound 2a or a pharmaceutically acceptable salt thereof by oral dosage form.

In some embodiments, the method includes administering Compound 1a or a pharmaceutically acceptable salt thereof by oral dosage form.

### Pharmaceutical Compositions and Formulations

In a further aspect, provided herein are pharmaceutical compositions. The pharmaceutical composition comprises any one (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a HGF/MET positive modulator, such as a compound of Formula (I), or a pharmaceutically acceptable salt, isotopic form, or stereoisomer thereof, and an additional therapeutic agent. Nonlimiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a HGF/MET positive modulator, such as a compound of Formula (I), or a pharmaceutically acceptable salt, isotopic form, or stereoisomer thereof, is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound of Formula (I), or a pharmaceutically acceptable salt, isotopic form, or stereoisomer thereof, is provided in the form of a rapid release formulation, in the form of an extended release formulation, or in the form of an intermediate release formulation. In yet other embodiments, the compound described herein is administered topically.

The HGF/MET positive modulator, such as the compounds of Formula (I), or a pharmaceutically acceptable salt, isotopic form, or stereoisomer thereof, are effective over a wide dosage range. The exact dosage will depend upon the route of administration, the form in which the compound is administered, the subject to be treated, the body weight of the subject to be treated, and the preference and experience of the attending physician.

In some embodiments, a HGF/MET positive modulator, such as a compound of Formula (I), or a pharmaceutically acceptable salt, isotopic form, or stereoisomer thereof, is administered in a single dose. Typically, such administration will be by injection, e.g., intravenous injection, in order to introduce the agent quickly. However, other routes are used as appropriate. A single dose of a compound of the disclosure may also be used for treatment of an acute condition (e.g., traumatic brain injury).

In some embodiments, a HGF/MET positive modulator, such as a compound of Formula (I), or a pharmaceutically acceptable salt, isotopic form, or stereoisomer thereof, is administered in multiple doses. In some embodiments, dosing is about once, twice, three times, four times, five times, six times, or more than six times per day. In other embodiments, dosing is about once a month, once every two weeks, once a week, or once every other day. In another embodiment a HGF/MET positive modulator, such as a compound of Formula (I), or a pharmaceutically acceptable salt, isotopic form, or stereoisomer thereof, and another therapeutic agent are administered together about once per day to about 6 times per day. In another embodiment the administration of a HGF/MET positive modulator, such as a compound of Formula (I), or a pharmaceutically acceptable salt, isotopic form, or stereoisomer thereof, and a therapeutic agent continues for less than about 7 days. In yet another embodiment the administration continues for more than about 6, 10, 14, 28 days, two months, six months, or one year. In some cases, continuous dosing is achieved and maintained as long as necessary.

Administration of the a HGF/MET positive modulator, such as the compounds of Formula (I), or a pharmaceutically acceptable salt, isotopic form, or stereoisomer thereof, may continue as long as necessary. In some embodiments, a compound of Formula (I), or a pharmaceutically acceptable salt, isotopic form, or stereoisomer thereof, is administered for more than 1, 2, 3, 4, 5, 6, 7, 14, or 28 days. In some embodiments, a compound of Formula (I), or a pharmaceutically acceptable salt, isotopic form, or stereoisomer thereof, is administered for less than 28, 14, 7, 6, 5, 4, 3, 2, or 1 day. In some embodiments, a compound of Formula (I), or a pharmaceutically acceptable salt, isotopic form, or stereoisomer thereof, is administered chronically on an ongoing basis, e.g., for the treatment of chronic effects (e.g., dementia).

In some embodiments, a HGF/MET positive modulator, such as the compound of Formula (I), or a pharmaceutically acceptable salt, isotopic form, or stereoisomer thereof, is administered in dosages. It is known in the art that due to intersubject variability in compound pharmacokinetics, individualization of dosing regimen is necessary for optimal therapy. Dosing for a compound may be found by routine experimentation in light of the instant disclosure.

In some embodiments, a HGF/MET positive modulator, such as the compounds Formula (I), or a pharmaceutically acceptable salt, isotopic form, or stereoisomer thereof, are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a HGF/MET positive modulator, such as a compound of Formula (I), or a pharmaceutically acceptable salt, isotopic form, or stereoisomer thereof, and a pharmaceutically acceptable diluent(s), excipient(s), or carrier(s). Also provided herein are methods for administering a pharmaceutical composition comprising a HGF/MET agonist, such as a compound of Formula (I), or a pharmaceutically acceptable salt, isotopic form, or stereoisomer thereof, and a pharmaceutically acceptable diluent(s), excipient(s), or carrier(s).

In certain embodiments, the compounds are administered as pharmaceutical compositions in which a HGF/MET positive modulator, such as compounds of Formula (I), or a pharmaceutically acceptable salt, isotopic form, or stereoisomer thereof, are mixed with other therapeutic agents, as in combination therapy. Encompassed herein are all combinations of active ingredients set forth in the methods section below and throughout this disclosure. In specific embodiments, the pharmaceutical compositions include one or more compounds of Formula (I), or a pharmaceutically acceptable salt, isotopic form, or stereoisomer thereof.

A pharmaceutical composition, as used herein, refers to a mixture of a HGF/MET positive modulator, such as a compound of Formula (I), or a pharmaceutically acceptable salt, isotopic form, or stereoisomer thereof, with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of a HGF/MET positive modulator, such as compounds of Formula (I), or a pharmaceutically acceptable salt, isotopic form, or stereoisomer thereof, provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more HGF/MET positive modulators, such as one or more compounds of Formula (I), or a pharmaceutically acceptable salt, isotopic form, or stereoisomer thereof, are formulated in an aqueous solutions. In specific embodiments, the aqueous solution is selected from, by way of example only, a physiologically compatible buffer, such as Hank's solution, Ringer's solution, or physiological saline buffer. In other embodiments, one or more compound of Formula (I), or a pharmaceutically acceptable salt, isotopic form, or stereoisomer thereof, are formulated for transmucosal administration. In specific embodiments, transmucosal formulations include penetrants that are appropriate to the barrier to be permeated (e.g., the blood-brain barrier). In still other embodiments wherein the compounds described herein are formulated for other parenteral injections, appropriate formulations include aqueous or non-aqueous solutions. In specific embodiments, such solutions include physiologically compatible buffers and/or excipients.

Suitable HGF/MET positive modulators, such as compounds of Formula (I) can be formulated for oral administration. Compounds are formulated by combining the active compounds with, e.g., pharmaceutically acceptable carriers or excipients. In various embodiments, the HGF/MET agonist, such as the compounds of Formula (I) or a pharmaceutically acceptable salt, isotopic form, or stereoisomer thereof, are formulated in oral dosage forms that include, by way of example only, tablets, powders, pills, dragees, capsules, liquids, gels, syrups, elixirs, slurries, suspensions, and the like.

In certain embodiments, pharmaceutical preparations for oral use are obtained by mixing one or more solid excipient with a HGF/MET positive modulator, such as one or more compounds of Formula (I), or a pharmaceutically acceptable salt, isotopic form, or stereoisomer thereof, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of a HGF/MET positive modulator, such as at least one of the compounds of Formula (I), or a pharmaceutically acceptable salt, isotopic form, or stereoisomer thereof, are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts a HGF/MET positive modulator, such as at least one of the compounds of Formula (I) or a pharmaceutically acceptable salt, isotopic form, or stereoisomer thereof, described herein are formulated for buccal or sublingual administration. Formulations suitable for buccal or sublingual administration include, by way of example only, tablets, lozenges, or gels. In still other embodiments, a HGF/MET positive modulator, such as the compounds of Formula (I), or a pharmaceutically acceptable salt, isotopic form, or stereoisomer thereof, are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, a suspension of an active compound or compounds (e.g., a HGF/MET positive modulator, such as the compounds of Formula (I), or a pharmaceutically acceptable salt, isotopic form, or stereoisomer thereof,) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, a HGF/MET positive modulator, such as the compounds of Formula (I), or a pharmaceutically acceptable salt, isotopic form, or stereoisomer thereof, are administered topically. The compounds are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, a HGF/MET positive modulator, such as the compounds of Formula (I), or a pharmaceutically acceptable salt, isotopic form, or stereoisomer thereof, are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of a HGF/MET positive modulator, such as the compounds of Formula (I), or a pharmaceutically acceptable salt, isotopic form, or stereoisomer thereof, is accomplished by means of iontophoretic patches and the like. In certain embodiments, transdermal patches provide controlled delivery of a HGF/MET positive modulator, such as the compounds of Formula (I), or a pharmaceutically acceptable salt, isotopic form, or stereoisomer thereof. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, a HGF/MET positive modulator, such a the compounds of Formula (I), or a pharmaceutically acceptable salt, isotopic form, or stereoisomer thereof, are formulated for administration by inhalation. Various forms suitable for administration by inhalation include, but are not limited to, aerosols, mists or powders. Pharmaceutical compositions of a HGF/MET positive modulator, such as any of compound of Formula (I), or a pharmaceutically acceptable salt, isotopic form, or stereoisomer thereof, are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the compound and a suitable powder base such as lactose or starch.

In still other embodiments, a HGF/MET positive modulator, such as the compounds of Formula (I), or a pharmaceutically acceptable salt, isotopic form, or stereoisomer thereof, are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a HGF/MET positive modulator, such as a compound of Formula (I), or a pharmaceutically acceptable salt, isotopic form, or stereoisomer thereof, are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and a HGF/MET positive modulator, such as at least one compound of Formula (I), or a pharmaceutically acceptable salt, isotopic form, or stereoisomer thereof, described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, a HGF/MET positive modulator, such as the compounds of Formula (I), or a pharmaceutically acceptable salt, isotopic form, or stereoisomer thereof, encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising a HGF/MET positive modulator, such as the compounds of Formula (I), or a pharmaceutically acceptable salt, isotopic form, or stereoisomer thereof, include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a HGF/MET positive modulator, such as a compound of Formula (I), or a pharmaceutically acceptable salt, isotopic form, or stereoisomer thereof. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising a HGF/MET positive modulator, such as at least one compound of Formula (I), or a pharmaceutically acceptable salt, isotopic form, or stereoisomer thereof, illustratively takes the form of a liquid where the agents are present in solution, in suspension or both. Typically, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a HGF/MET positive modulator, such as a compound of Formula (I), or a pharmaceutically acceptable salt, isotopic form, or stereoisomer thereof. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite, and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, a HGF/MET positive modulator, such as the compounds of Formula (I), or a pharmaceutically acceptable salt, isotopic form, or stereoisomer thereof, are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, I about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of a HGF/MET positive modulator, such as the compound of Formula (I), provided in the pharmaceutical compositions of the present disclosure is less than 100%, 90%, 80%, 70%, 60%, 50%, 40%, 30%, 20%, 19%, 18%, 17%, 16%, 15%, 14, 13%, 12%, 11%, 10% 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of a HGF/MET positive modulator, such as the compound of Formula (I), provided in the pharmaceutical compositions of the present disclosure is greater than 90%, 80%, 70%, 60%, 50%, 40%, 30%, 20%, 19.75%, 19.50%, 19.25%, 19%, 18.75%, 18.50%, 18.25%, 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75% 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4% 3.75%, 3.50%, 3.25%, 3%, 2.75%, 2.50%, 2.25%, 2%, 1.75%, 1.50%, 125% 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of a HGF/MET positive modulator, such as the compound of Formula (I), or a pharmaceutically acceptable salt, isotopic form, or stereoisomer thereof, provided in the pharmaceutical compositions ranges from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, or approximately 1% to approximately 10% w/w, w/v or v/v.

In some embodiments, the concentration of a HGF/MET positive modulator, such as the compound of Formula (I), or a pharmaceutically acceptable salt, isotopic form, or stereoisomer thereof, provided in the pharmaceutical compositions ranges from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, or approximately 0.1% to approximately 0.9% w/w, w/v or v/v.

In some embodiments, the amount of a HGF/MET positive modulator, such as the compound of Formula (I), or a pharmaceutically acceptable salt, isotopic form, or stereoisomer thereof, provided in the pharmaceutical compositions is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of a HGF/MET positive modulator, such as the compound of Formula (I), or a pharmaceutically acceptable salt, isotopic form, or stereoisomer thereof, provided in the pharmaceutical compositions of the present disclosure is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of a HGF/MET positive modulator, such as the compound of Formula (I), or a pharmaceutically acceptable salt, isotopic form, or stereoisomer thereof, provided in the pharmaceutical compositions ranges from 0.0001-10 g, 0.0005-9 g, 0.001-8 g, 0.005-7 g, 0.01-6 g, 0.05-5 g, 0.1-4 g, 0.5-4 g, or 1-3 g.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that these examples are exemplary and not exhaustive. Many changes can be made in the specific embodiments which are disclosed and still obtain a like or similar result without departing from the spirit and scope of the disclosure.

The chemical reactions in the Synthetic Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be performed by modifications apparent to those skilled in the art, for example by appropriately protecting interfering groups, by utilizing other suitable reagents known in the art other than those described, or by making routine modification of reaction conditions, reagents, and starting materials. Alternatively, other reactions disclosed herein or known in the art will be recognized as having applicability for preparing other compounds of the present disclosure.

Unless indicated otherwise in the following Examples, the compounds are isolated as a racemic mixture.

The following abbreviations may be relevant for the application.

### Abbreviations

AcOH: acetic acid
CAN: ceric ammonium nitrate
DAST: diethylaminosulfur trifluoride
DCM: dichloromethane
DIPEA: N,N-diisopropylethylamine
DMEM: Dulbecco's Modified Eagle Medium
DMF: dimethylformamide
DMSO: dimethylsulfoxide
EMEM: Eagle's Minimum Essential Medium
EtOAc: ethyl acetate
EtOH: ethanol
FBS: fetal bovine serum
Fmoc: fluorenylmethoxycarbonyl
HATU: (1-[bis(dimethylamino)methylene]-1H-1,2,3-triazolo[4,5-b]pyridinium 3-oxide hexafluorophosphate
LC/MS: liquid chromatography-mass spectrometry
Me: methyl
MeOH: methanol
PBS: phosphate buffered saline
Pic-BH₃: picoline borane
PMB: para-methoxybenzyl ether
Prep HPLC: preparative high performance liquid chromatography
rt or RT: room temperature
TFA: trifluoroacetic acid
TLC: thin layer chromatography
T₃P: Propanephosphonic acid anhydride

### SYNTHETIC EXAMPLES

### Example S1. Synthesis of (6S)-6-methyl-8-(2-methylbutyl)hexahydro-4H-pyrazino[1,2-a]pyrimidine-4,7(6H)-dione

The synthetic route for preparing this starting material compound is shown in Scheme 1.

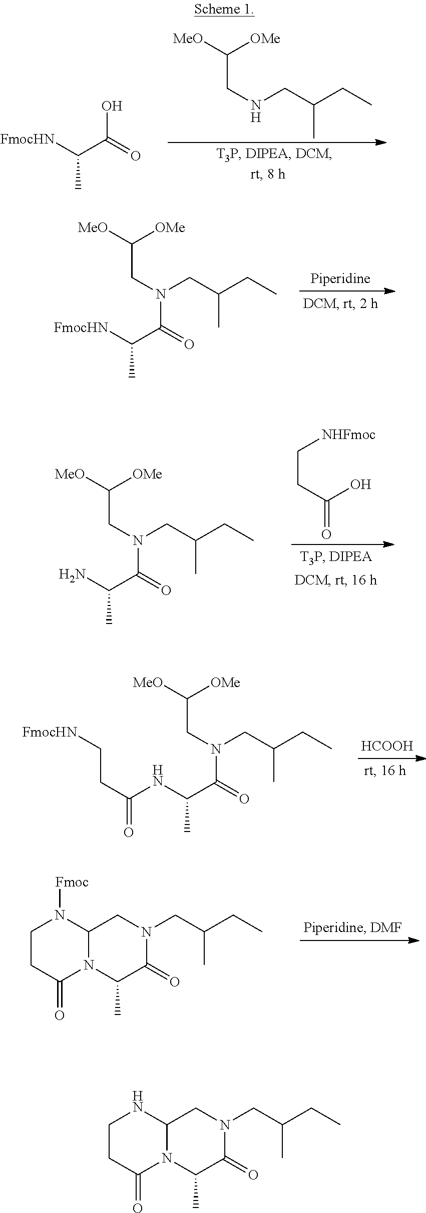

Step 1: Synthesis of (9H-fluoren-9-yl)methyl (2S)-1-((2,2-dimethoxyethyl)(2-methylbutyl)amino)-1-oxopropan-2-ylcarbamate. To a stirred solution of compound (S)-2-(((9H-fluoren-9-yl)methoxy)carbonylamino)propanoic acid (5.0 g, 16.07) in dichloromethane (100 mL) was added T₃P (15.2 mL, 24.1) and DIPEA (5.6 mL, 32.1 mmol) at room temperature. The reaction mixture was stirred at room temperature for 15 min and N-(2,2-dimethoxyethyl)-2-methylbutan-1-amine (2.81 g, 32.1 mmol.) was added, and stirring was continued at room temperature for 8 hours. The reaction was monitored by TLC. After completion, the reaction mixture was quenched with ice cold water (100 mL) and extracted with dichloromethane (2×100 mL). The combined organic layers were dried over anhydrous Na₂SO₄, filtered and concentrated under reduced pressure to give crude compound. The crude compound was purified by flash column chromatography (100-200 mesh silica gel, eluted with 40% ethyl acetate in petroleum ether) to afford pure compound (9H-fluoren-9-yl)methyl (2S)-1-((2,2-dimethoxyethyl)(2-methylbutyl)amino)-1-oxopropan-2-ylcarbamate (5.2 g, 69.1%) as a gummy compound.

Step 2: Synthesis of (2S)-2-amino-N-(2,2-dimethoxyethyl)-N-(2-methylbutyl)propenamide. To a stirred solution of (9H-fluoren-9-yl)methyl (2S)-1-((2,2-dimethoxyethyl)(2-methylbutyl)amino)-1-oxopropan-2-ylcarbamate (34.0 g, 72.6 mmol) in DMF (230 mL) was added 20% piperidine in DMF (70 mL) at 0° C. The reaction mixture was stirred at room temperature for 2 hours. The reaction was monitored by TLC. After completion of the reaction, excess DMF (100 mL) was added, then washed with excess n-hexane (3×200 mL). The DMF layer was collected and poured in ice cold water (1000 mL), then extracted with 10% methanol-dichloromethane (3×500 mL). The combined organic layers was dried over anhydrous Na₂SO₄, filtered and concentrated under reduced pressure to give (2S)-2-amino-N-(2,2-dimethoxyethyl)-N-(2-methylbutyl)propanamide (20.4 g, 68.4%) as a gummy solid.

Step 3: Synthesis of (9H-fluoren-9-yl)methyl3-((2S)-1-((2,2-dimethoxyethyl)(2-methylbutyl)amino)-1-oxopropan-2-ylamino)-3-oxopropylcarbamate. To a stirred solution of 3-(((9H-fluoren-9-yl) methoxy)carbonylamino)propanoic acid (20.2 g, 81.2 mmol) stirred in dichloromethane at room temperature (500 mL) was added T₃P (80 mL, 121.8 mmol) and DIPEA (28.6 mL, 160.4 mmol), and the mixture was stirred for 10 minutes. To this (2S)-2-amino-N-(2,2-dimethoxyethyl)-N-(2-methylbutyl)propanamide (25.53 81.2 mmol) was added and stirring was continued at room temperature for 16 hours. The reaction progress was monitored by TLC. After completion, the reaction mixture was quenched with water (500 mL) and the mixture was extracted with dichloromethane (2×500 mL). The combined organic layers were dried over Na₂SO₄, filtered and concentrated under reduced pressure to give crude product. The crude compound was purified by flash column chromatography (100-200 mesh Silica gel, eluted with 70% ethyl acetate in petroleum ether) to afford pure compound (9H-fluoren-9-yl)methyl3-((2S)-1-((2,2-dimethoxyethyl)(2-methylbutyl)amino)-1-oxopropan-2-ylamino)-3-oxopropylcarbamate (21.2 g, 78.6%) as a gummy compound.

Step 4: Synthesis of (6S)-(9H-fluoren-9-yl)methyl 6-methyl-8-(2-methylbutyl)-4,7-dioxooctahydro-1H-pyrazino[1,2-a]pyrimidine-1-carboxylate. To a stirred solution of (9H-fluoren-9-yl)methyl 3-((2S)-1-((2,2-dimethoxyethyl)(2-methylbutyl)amino)-1-oxopropan-2-ylamino)-3-oxopropylcarbamate (21.0 g, 38.9 mmol) was added formic acid (105 mL). The reaction mixture was stirred at room temperature for 12 hours. The reaction progress was monitored by TLC. After completion, the reaction mixture was concentrated under reduced pressure to give crude compound. The crude compound was taken up in saturated aqueous NaHCO₃ (200 mL) solution, then extracted with ethyl acetate (3×500 mL). The combined organic layers were washed with brine solution (500 mL), then the combined organic layers were dried over anhydrous Na₂SO₄, filtered and concentrated under reduced pressure. The crude compound was purified by flash column chromatography (100-200 mesh silica gel, eluted with 50% ethyl acetate in petroleum ether) to afford pure compound (6S)-(9H-fluoren-9-yl)methyl 6-methyl-8-(2-methylbutyl)-4,7-dioxooctahydro-1H-pyrazino[1,2-a]pyrimidine-1-carboxylate (25 g, 69.0%) as a gum.

Step 5: Synthesis of (6S)-6-methyl-8-(2-methylbutyl)tetrahydro-1H-pyrazino[1,2-a]pyrimidine-4,7(6H, 8H)-dione. To a stirred solution of (6S)-(9H-fluoren-9-yl)methyl 6-methyl-8-(2-methylbutyl)-4,7-dioxooctahydro-1H-pyrazino[1,2-a]pyrimidine-1-carboxylate (14.0 g, 29.4 mmol) at 0° C. in DMF (70 mL) was added 20% piperidine in DMF (30 mL). The reaction mixture was allowed to warm to room temperature and stirred for 2 hours. The reaction was monitored by TLC. After complete consumption of starting material, additional DMF was added (50 mL), then the mixture was washed with excess n-hexane (3×200 mL). The DMF layer was poured into ice cold water (1000 mL) and extracted with 10% methanol-dichloromethane (3×500 mL). The combined organic layers were dried over anhydrous Na₂SO₄, filtered and concentrated under reduced pressure to provide the desired crude compound (6S)-6-methyl-8-(2-methylbutyl)tetrahydro-1H-pyrazino[1,2-a]pyrimidine-4,7(6H, 8H)-dione (6.25 g, 83.8%) as a solid.

### Example S2. Synthesis of Compound 1a

The synthetic route for preparing Compound 1a is shown in Scheme 2.

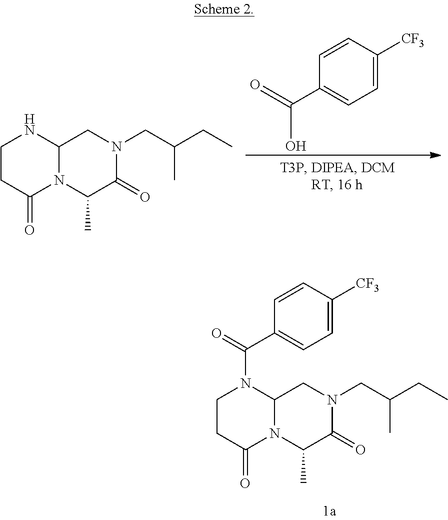 [SMILES: CCC(C)CN1CC2NCCC(=O)N2[C@@H](C)C1=O ; O=C(O)c1ccc(C(F)(F)F)cc1 ; CCC(C)CN1CC2N(C(=O)c3ccc(C(F)(F)F)cc3)CCC(=O)N2[C@@H](C)C1=O]

To a solution of 4-(trifluoromethyl)benzoic acid (0.232 g, 0.91 mmol) stirred in dichloromethane (20 mL) at room temperature was added T₃P (1.2 mL, 1.37 mmol) and DIPEA (0.42 mL, 1.82 mmol), and the mixture was stirred for 15 minutes. To this (6S)-6-methyl-8-(2-methylbutyl)tetrahydro-1H-pyrazino[1,2-a]pyrimidine-4,7(6H, 8H)-dione (0.310 g, 0.91 mmol) was added and stirring was continued for 8 hours. The reaction progress was monitored by TLC. After reaction completion, the mixture was quenched with water (50 mL) and extracted with dichloromethane (2×50 mL). The combined organic layers were dried over Na₂SO₄, filtered and concentrated under reduced pressure. The crude compound was purified by Prep HPLC. The pure fractions were combined and concentrated under reduced pressure, then lyophilized to afford 1a (0.340 g, 65.3%) as a solid. Prep HPLC method: Mobile phase A: 10 mM ammonium bicarbonate in water; Mobile phase B: acetonitrile; Column: X-Select phenyl hexyl (150×19 mm 5μ); Flow: 16 mL/min. MS (ESI) m/z [M+H]⁺: 426.05.

### Example S3. Synthesis of Compound 2a

The synthetic route for preparing Compound 2a is shown in Scheme 3.

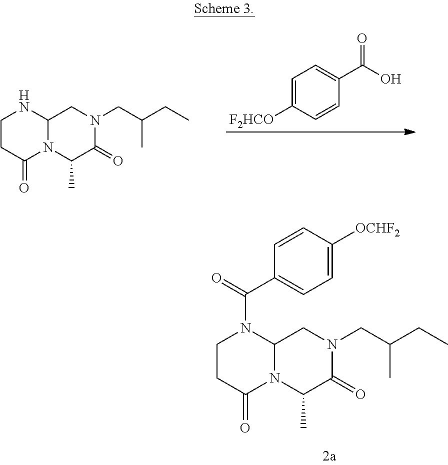 [SMILES: CCC(C)CN1CC2NCCC(=O)N2[C@@H](C)C1=O ; O=C(O)c1ccc(OC(F)F)cc1 ; CCC(C)CN1CC2N(C(=O)c3ccc(OC(F)F)cc3)CCC(=O)N2[C@@H](C)C1=O]

To a solution of 4-(difluoromethoxy) benzoic acid (0.37 g, 1.968 mmol) in dichloromethane (15 mL) at room temperature was added DIPEA (0.8 ml, 5.904 mmol) and T₃P (2.0 mL, 3.936 mmol). The mixture was stirred for 30 min, then (6S)-6-methyl-8-(2-methylbutyl)hexahydro-4H-pyrazino[1,2-a]pyrimidine-4,7(6H)-dione (0.4 g, 1.578 mmol) was added, and stirring was continued for 16 hours. Progress of the reaction was monitored by TLC and LC/MS. The reaction mixture was diluted with dichloromethane (100 mL) and washed with water (50 mL) and saturated sodium chloride solution (50 mL), then dried over sodium sulfate, filtered and concentrated under reduced pressure. The crude product was purified by Prep HPLC. The pure fractions were collected and lyophilized to afford 2a (380 mg 46%) as a solid. Prep HPLC condition: Mobile phase A: 10 mM ammonium bicarbonate in water; Mobile phase B: Acetonitrile; Column: Kromosil phenyl (150×25 mm 10μ); Flow: 25 mL/min. MS (ESI) m/z [M+H]⁺: 424.11.

### Example S4. Synthesis of Compound 3a

The synthetic route for preparing Compound 3a is shown in Scheme 4.

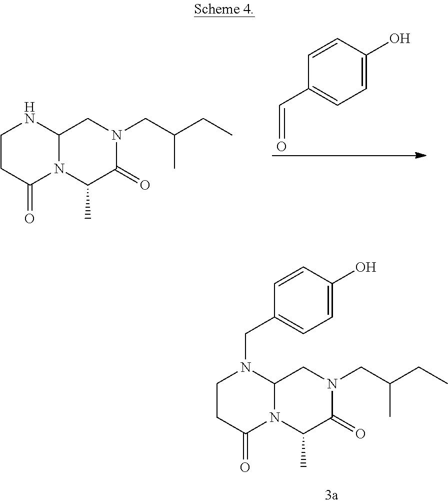 [SMILES: CCC(C)CN1CC2NCCC(=O)N2[C@@H](C)C1=O ; O=Cc1ccc(O)cc1 ; CCC(C)CN1CC2N(Cc3ccc(O)cc3)CCC(=O)N2[C@@H](C)C1=O]

To a solution of (6S)-6-methyl-8-(2-methylbutyl)tetrahydro-1H-pyrazino[1,2-a]pyrimidine-4,7(6H, 8H)-dione (0.500 g, 1.97 mmol) stirred in methanol (20 mL) at room temperature was added 4-hydroxybenzaldehyde (0.289 g, 1.97 mmol) and acetic acid (0.23 mL, 3.95 mmol). The reaction mixture was stirred at room temperature for 5 minutes. To this picoline borane (0.253 g, 2.37 mmol) was added, and stirring was continued for 48 hr. The reaction progress was monitored by TLC. After completion, the reaction mixture was quenched with ice cold water (50 mL), and the mixture was extracted with 10% methanol-dichloromethane (3×40 mL). The combined organic layers were dried over Na₂SO₄, filtered and concentrated under reduced pressure. The crude compound was purified by Prep HPLC. The pure fractions were combined and concentrated under reduced pressure, then lyophilized to give 3a (0.180 g, 46.09%) as a solid. Prep HPLC Method: Mobile Phase A: 10 mM ammonium bicarbonate in water; Mobile Phase B: Acetonitrile; Column: Kromosil Phenyl (150×25 mm 10μ); Flow: 25 mL/min. MS (ESI) m/z [M+H]⁺: 360.11.

### Example S5. Synthesis of Compound 4a

The synthetic route for preparing Compound 4a is shown in Scheme 5.

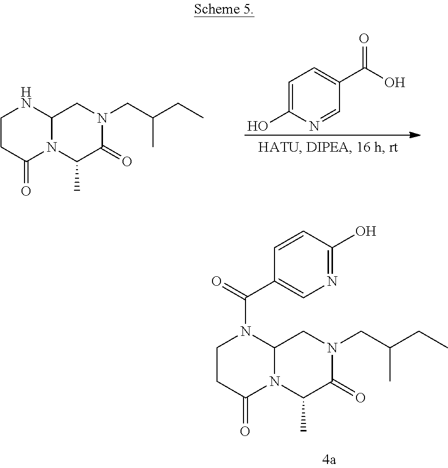 [SMILES: CCC(C)CN1CC2NCCC(=O)N2[C@@H](C)C1=O ; O=C(O)c1ccc(O)nc1 ; CCC(C)CN1CC2N(C(=O)c3ccc(O)nc3)CCC(=O)N2[C@@H](C)C1=O]

To a solution of 6-hydroxynicotinic acid (0.340 g 2.446 mmol) in DMF (15 mL) at room temperature was added DIPEA (1.30 mL, 7.338 mmol) and HATU (1.39 g, 3.669 mmol). The resulting reaction mixture was stirred for 30 min, then (6S)-6-methyl-8-(2-methylbutyl)hexahydro-4H-pyrazino[1,2-a]pyrimidine-4,7(6H)-dione (0.495 g, 1.956 mmol.) was added, and the mixture was stirred for 16 hours. Progress of the reaction was monitored by TLC and LC/MS (TLC system: 10% methanol/dichloromethane, Rf 0.15, Detection: UV). The reaction mixture was quenched with cold water (100 mL) and extracted with 10% methanol/dichloromethane (3×100 mL). The combined organic layers were washed with cold water (50 mL) and cold brine solution (50 mL), dried over sodium sulfate, filtered and concentrated under reduced pressure. The crude product was purified by Prep HPLC. The pure fractions were collected and lyophilized to afford 4a (160 mg, 21.8%) as a solid. Prep HPLC Method: Mobile Phase A: 0.01 mM ammonium bicarbonate in water; Mobile Phase B: acetonitrile; Column: X-Select phenyl hexyl (150×19 mm, 5μ); Flow: 15 mL/min. MS (ESI) m/z [M+H]⁺: 375.05.

### Example S6. Synthesis of Compound 5a

The synthetic route for preparing Compound 5a is shown in Scheme 6.

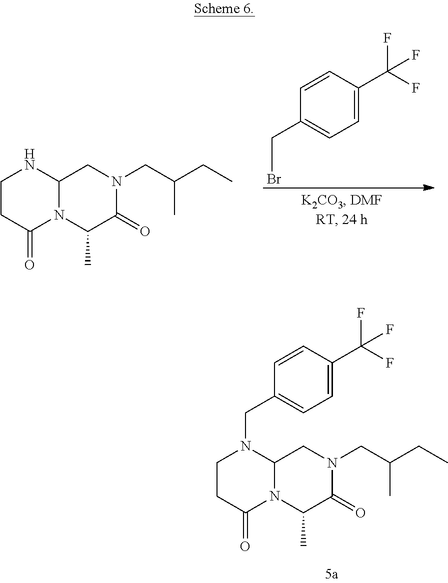

To a solution of (6S)-6-methyl-8-(2-methylbutyl)tetrahydro-1H-pyrazino[1,2-a]pyrimidine-4,7(6H, 8H)-dione (0.5 g, 1.97 mmol) and 1-(bromomethyl)-4-(trifluoromethyl)benzene (0.470 g, 1.97 mmol) stirred in DMF (20 mL) at room temperature was added K₂CO₃ (0.546 g, 3.95 mmol), and the mixture was stirred for 8 hr. The reaction progress was monitored by TLC. After completion, the mixture was quenched with water (100 mL) and extracted with ethyl acetate (3×50 mL). The combined organic layers were dried over Na₂SO₄, filtered and concentrated under reduced pressure. The crude compound was purified by Prep HPLC. The pure fractions were combined and concentrated under reduced pressure, then lyophilized to afford 5a (0.270 g, 63.8%) as a gum. Prep HPLC Method: Mobile Phase A: 10 mM ammonium bicarbonate in water; Mobile Phase B: Acetonitrile; Column: Kromosil Cis (150×25 mm 10μ); Flow: 25 mL/min. MS (ESI) m/z [M+H]⁺: 412.2.

### Example S7. Synthesis of Compound 6a

The synthetic route for preparing Compound 6a is shown in Scheme 7.

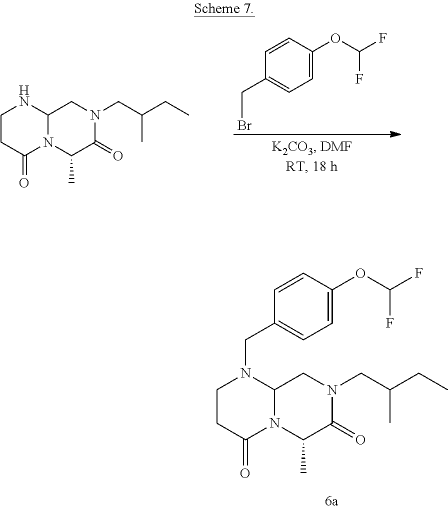 [SMILES: CCC(C)CN1CC2NCCC(=O)N2[C@@H](C)C1=O ; FC(F)Oc1ccc(CBr)cc1 ; CCC(C)CN1CC2N(Cc3ccc(OC(F)F)cc3)CCC(=O)N2[C@@H](C)C1=O]

To a solution of (6S)-6-methyl-8-(2-methylbutyl)tetrahydro-1H-pyrazino[1,2-a]pyrimidine-4,7(6H, 8H)-dione (0.500 g, 1.97 mmol) and 1-(bromomethyl)-4-(difluoromethoxy)benzene (0.466 g, 1.97 mmol) stirred in DMF (20 mL) at room temperature was added K₂CO₃ (0.546 g, 9.95 mmol). The reaction mixture was stirred at room temperature for 18 hr. The reaction progress was monitored by TLC. After completion, the reaction mixture was quenched with water (100 mL) and extracted with ethyl acetate (3×50 mL). The combined organic layers were dried over Na₂SO₄, filtered and concentrated under reduced pressure. The crude compound was purified by Prep HPLC. The pure fractions were combined and concentrated under reduced pressure, then lyophilized to afford 6a (0.178 g, 41.5%) as a semi-solid. Prep HPLC Method: Mobile Phase A: 10 mM ammonium bicarbonate in water; Mobile Phase B: acetonitrile; Column: X-Select Cis (250×19 mm, 5μ); Flow: 18 mL/min. MS (ESI) m/z [M+H]⁺: 410.11.

### Example S8. Synthesis of Compound 7a

The synthetic route for preparing Compound 7a is shown in Scheme 8.

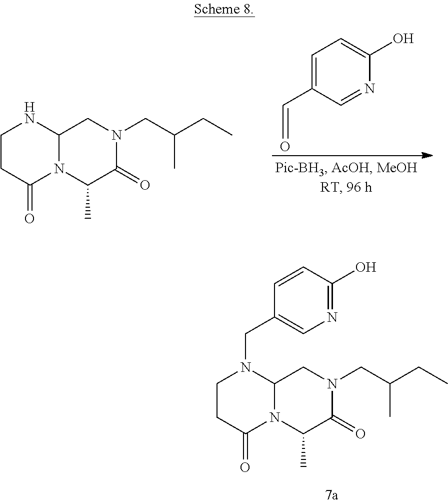 [SMILES: CCC(C)CN1CC2NCCC(=O)N2[C@@H](C)C1=O ; O=Cc1ccc(O)nc1 ; CCC(C)CN1CC2N(Cc3ccc(O)nc3)CCC(=O)N2[C@@H](C)C1=O]

To a solution of compound (6S)-6-methyl-8-(2-methylbutyl)tetrahydro-1H-pyrazino[1,2-a]pyrimidine-4,7(6H, 8H)-dione (0.500 g, 1.97 mmol) stirred in methanol (20 mL) at room temperature was added 6-hydroxynicotinaldehyde (0.243 g, 1.97 mmol) and acetic acid (0.25 mL, 3.95 mmol), and the mixture was stirred for 5 min. To this picoline borane (0.318 g, 2.96 mmol) was added and stirring was continued for 96 hours. The reaction progress was monitored by TLC. After completion, the reaction mixture was quenched with ice cold water (50 mL) and extracted with 10% methanol-dichloromethane (3×40 mL). The combined organic layers were dried over Na₂SO₄, filtered and concentrated under reduced pressure. The crude compound was purified by Prep HPLC. The pure fractions were collected and concentrated under reduced pressure, then lyophilized to afford 7a (0.164 g, 42%) as a solid. Prep HPLC Method: Mobile Phase A: 10 mM ammonium bicarbonate in water; Mobile Phase B: acetonitrile; Column: X-BRIDGE Cis (250×19 mm, 5μ); Flow: 18 mL/min. MS (ESI) m/z [M+H]⁺: 361.11.

### Example S9. Synthesis of Compound 8a

The synthetic route for preparing Compound 8a is shown in Scheme 9.

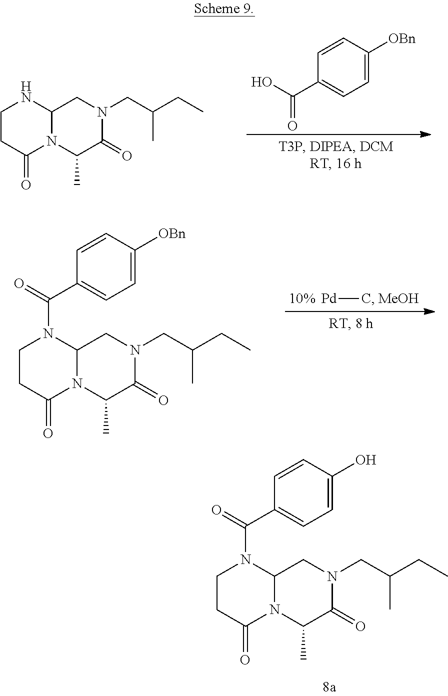

Step 1: Synthesis of (6S)-1-(4-(benzyloxy)benzoyl)-6-methyl-8-(2-methylbutyl)hexahydro-4H-pyrazino[1,2-a]pyrimidine-4,7(6H)-dione. To a solution of 4-(benzyloxy)benzoic acid (0.360 g, 1.42 mmol) stirred in dichloromethane (20 mL) at room temperature was added T₃P (1.2 mL, 1.7 mmol) and DIPEA (0.55 mL, 2.84 mmol), and the mixture was stirred for 15 min. To this (6S)-6-methyl-8-(2-methylbutyl)tetrahydro-1H-pyrazino[1,2-a]pyrimidine-4,7(6H, 8H)-dione (0.400 g, 1.42 mmol) was added, and stirring was continued at room temperature for 16 hours. The reaction progress was monitored by TLC. After completion, the reaction mixture was quenched with water (50 mL) and extracted with dichloromethane (2×50 mL). The combined organic layers were dried over Na₂SO₄, filtered and concentrated under reduced pressure to give 0.9 g of crude material. Analysis of the crude material by LC/MS showed 54.59% of the desired product. The crude material was used in the next step without purification.

Step 2: Synthesis of Compound 8a. To a solution of (6S)-1-(4-(benzyloxy)benzoyl)-6-methyl-8-(2-methylbutyl)tetrahydro-1H-pyrazino[1,2-a]pyrimidine-4,7(6H, 8H)-dione (0.900 g) stirred in methanol (20 mL) at room temperature was added 10% Pd—C(0.200 g), under N₂ atmosphere. The reaction mixture was stirred at room temperature under an H₂ balloon for 8 hr. The reaction progress was monitored by TLC. After completion, the reaction mixture was filtered through Celite and evaporated under reduced pressure to afford the crude compound. The crude compound was dissolved in dichloromethane (50 mL), washed with aqueous NaHCO₃ solution (20 mL) and brine solution (20 mL). The filtrate was dried over Na₂SO₄, filtered and concentrated under reduced pressure. The crude compound was triturated with diethyl ether to afford 8a (0.330 g, 82%) as a solid. MS (ESI) m/z [M+H]⁺: 374.11.

### Example S10. Synthesis of Compound 9

The synthetic route for preparing Compound 9 is shown in Scheme 10.

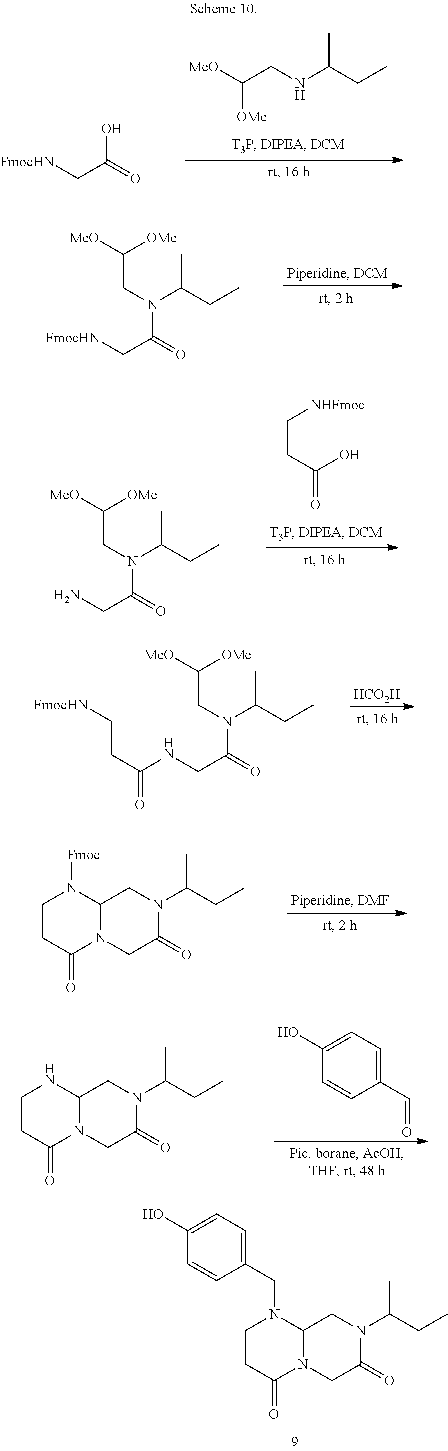

Step 1: Synthesis of (9H-fluoren-9-yl)methyl 2-(sec-butyl(2,2-dimethoxyethyl)amino)-2-oxoethylcarbamate. To a stirred solution of 2-(((9H-fluoren-9-yl)methoxy)carbonylamino)acetic acid (10 g, 33.6 mmol) in dichloromethane (100 mL), cooled to 0° C. were added DIPEA (11.88 mL, 67.3 mmol), N-(2,2-dimethoxyethyl)butan-2-amine (10.84 g, 67.3 mmol) and T₃P (53.0 mL, 84.1 mmol), and the reaction mixture was stirred for 16 hours at room temperature. Reaction progress was monitored by TLC. After completion of the reaction, ice cold water (100 mL) was added and extracted with ethyl acetate (2×150 mL). The combined organic layers were dried over anhydrous Na₂SO₄, filtered and concentrated under reduced pressure to obtain the desired crude product. The crude compound was purified by flash column chromatography (100-200 mesh silica gel) and eluted with 20-25% ethyl acetate in petroleum ether to afford (9H-fluoren-9-yl)methyl 2-(sec-butyl(2,2-dimethoxyethyl)amino)-2-oxoethylcarbamate (10.8 g, 72.9%) as a solid.

Step 2: Synthesis of 2-amino-N-sec-butyl-N-(2,2-dimethoxyethyl)acetamide. To a solution of (9H-fluoren-9-yl)methyl 2-(sec-butyl(2,2-dimethoxyethyl)amino)-2-oxoethylcarbamate (10.8 g, 24.5 mmol) in DMF (20 mL), cooled to 0° C., was added piperidine (2.4 mL) and the reaction mixture was stirred at room temperature for 2 hours. Progress of the reaction was monitored by TLC. After TLC indicated complete consumption of starting material, the reaction mixture was diluted with petroleum ether (2×100 mL), then water was added and the mixture was separated. The aqueous layer was extracted with dichloromethane (2×150 mL). The combined organic layers were dried over anhydrous Na₂SO₄, filtered and concentrated under reduced pressure to obtain the desired pure product 2-amino-N-sec-butyl-N-(2,2-dimethoxyethyl)acetamide (3.6 g, 67.2%) as a solid.

Step 3: Synthesis of (9H-fluoren-9-yl)methyl-3-(2-(sec-butyl(2,2-dimethoxyethyl)amino)-2-oxoethylamino)-3-oxopropylcarbamate. To a stirred solution of 2-amino-N-sec-butyl-N-(2,2-dimethoxyethyl)acetamide (3.6 g, 16.5 mmol) in dichloromethane (40 mL) were added DIPEA (31.91 mL, 49.5 mmol), 3-(((9H-fluoren-9-yl)methoxy)carbonylamino)propanoic acid (5.14 g, 16.5 mmol) and T₃P (39.13 g, 33 mmol) at 0° C. The reaction mixture was stirred at room temperature for 16 hours. Progress of the reaction was monitored by TLC. After completion of the reaction, the reaction water (100 mL) was added and the organic phase was separated. The aqueous phase was extracted with dichloromethane (2×150 mL). The combined organic layers were dried over Na₂SO₄, filtered and concentrated under reduced pressure to give the crude product. The crude product was purified by column chromatography using silica (230-400 mesh; 23-25% ethyl acetate/petroleum ether as eluent). Collected pure fractions were concentrated under reduced pressure to give (9H-fluoren-9-yl)methyl-3-(2-(sec-butyl(2,2-dimethoxyethyl)amino)-2-oxoethylamino)-3-oxopropylcarbamate (4.1 g, 48.6%) as a gum.

Step 4: Synthesis of (9H-fluoren-9-yl)methyl 8-sec-butyl-4,7-dioxooctahydro-1H-pyrazino[1,2-a]pyrimidine-1-carboxylate. To a solution of (9H-fluoren-9-yl)methyl-3-(2-(sec-butyl(2,2-dimethoxyethyl)amino)-2-oxoethylamino)-3-oxopropylcarbamate (4.1 g, 8.01 mmol) in acetic acid (2 mL) was stirred for 16 hours at room temperature. Progress of the reaction was monitored by TLC. After TLC indicated complete consumption of the starting material, the reaction mixture was concentrated and the resulting mass was diluted with water and extracted with dichloromethane (2×100 mL). The combined organic layers were dried over Na₂SO₄, filtered and concentrated under reduced pressure to give the product (9H-fluoren-9-yl)methyl 8-sec-butyl-4,7-dioxooctahydro-1H-pyrazino[1,2-a]pyrimidine-1-carboxylate. (3.2 g, 89.3%) as a gum.

Step 5: Synthesis of 8-sec-butyltetrahydro-1H-pyrazino[1,2-a]pyrimidine-4,7(6H, 8H)-dione. To a solution of (9H-fluoren-9-yl)methyl 8-sec-butyl-4,7-dioxooctahydro-1H-pyrazino[1,2-a]pyrimidine-1-carboxylate (3.2 g, 7.1 mmol) in DMF (20 mL), cooled to 0° C., was added piperidine (0.7 mL, 1.0 eq) and the reaction mixture was stirred at room temperature for 2 hours. Progress of the reaction was monitored by TLC. After TLC indicated complete consumption of starting material, the reaction mixture was washed with petroleum ether (2×50 mL) to remove the non-polar impurities. Cold water was added and extracted with dichloromethane (2×100 mL). The combined organic layers were dried over Na₂SO₄, filtered and concentrated under reduced pressure to give the pure product 8-sec-butyltetrahydro-1H-pyrazino[1,2-a]pyrimidine-4,7(6H, 8H)-dione (900 mg, 55.9%) as a solid.

Step 6: Synthesis of Compound 9. To a stirred solution of (8-(sec-butyl)hexahydro-4H-pyrazino[1,2-a]pyrimidine-4,7(6H)-dione (0.500 g, 2.2 mmol) and 4-hydroxybenzaldehyde (0.271 g, 2.2 mmol) in methanol (10 mL) was added acetic acid (0.27 mL, 2.0 eq.) and picoline borane (0.285 g, 2.6 mmol) at room temperature. The reaction mixture was stirred at room temperature for 48 hr. The reaction progress was monitored by TLC. After completion, the reaction mixture was quenched with ice cold water (10 mL) and extracted with ethyl acetate (2×20 mL). The combined organic layers were dried over Na₂SO₄, filtered and concentrated under reduced pressure to give crude product. The crude compound was analyzed by LC/MS. The crude LC/MS data showed 8.28% desired mass. The crude compound was purified by column chromatography over silica gel (100-200), and 50-70% ethyl acetate in petroleum ether eluted the desired compound. The LC/MS of the eluted fractions showed 72.16% desired mass, which was further purified by Prep HPLC. After Prep HPLC purification, the fractions were collected and concentrated under reduced pressure, then lyophilized to afford 9 (0.168 g, 22.8%) as a solid. Prep HPLC Method: Mobile Phase A: 10 mM ammonium bicarbonate in water; Mobile Phase B: acetonitrile; Column: X-BRIDGE Cis (150×19 mm 5μ); Flow: 18 mL/min. MS (ESI) m/z [M+H]⁺: 332.2.

### Example S11. Synthesis of Compound 10

The synthetic route for preparing Compound 10 is shown in Scheme 11.

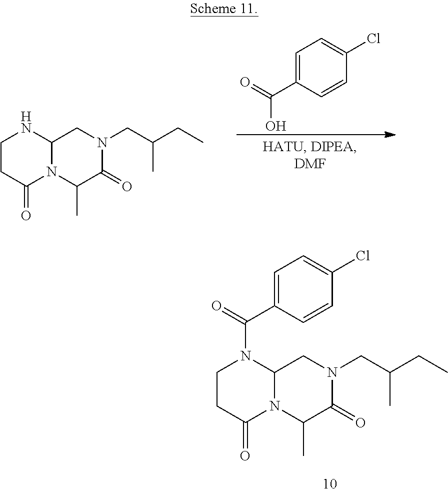 [SMILES: O=C(O)c1ccc(Cl)cc1 ; CCC(C)CN1CC2NCCC(=O)N2C(C)C1=O ; CCC(C)CN1CC2N(C(=O)c3ccc(Cl)cc3)CCC(=O)N2C(C)C1=O]

To a solution of 6-methyl-8-(2-methylbutyl)hexahydro-4H-pyrazino[1,2-a]pyrimidine-4,7(6H)-dione (250 mg, 0.98 mmol) and 4-chlorobenzoic acid (170 mg, 1.09 mmol) in DMF (4 mL) at 0° C. was added HATU (413 mg, 1.08 mmol) followed by DIPEA (0.35 mL, 1.97 mmol). The reaction mixture was stirred at room temperature for 12 h. After completion, the reaction mixture was quenched with ice cold water (50 mL) and the aqueous layer was extracted with EtOAc (50 mL×2). The organic layer was washed with cold H₂O (30 mL) followed by saturated brine (30 mL), dried over Na₂SO₄ and concentrated under reduced pressure. The crude obtained was purified by column chromatography (Silica 100-200 mesh; 30% EtOAc in hexanes) to afford 1-(4-chlorobenzoyl)-6-methyl-8-(2-methylbutyl)hexahydro-4H-pyrazino[1,2-a]pyrimidine-4,7(6H)-dione 10 (150 mg, 0.383 mmol, 39.2% yield) as a solid. MS (ESI) m/z [M+H]⁺: 392.05. ¹H NMR (400 MHz, DMSO-d₆) δ 0.66-0.89 (m, 6H) 0.91-1.42 (m, 4H) 1.57-1.78 (m, 1H) 2.16-2.35 (m, 2H) 2.55-2.65 (m, 2H) 3.08-3.23 (m, 2H) 3.28-3.40 (m, 1H) 3.51-3.64 (m, 2H) 4.76-4.89 (m, 1H) 5.88-6.02 (m, 1H) 7.46-7.56 (m, 4H).

### Example S12. Synthesis of Compound 11

The synthetic route for preparing Compound 11 is shown in Scheme 12.

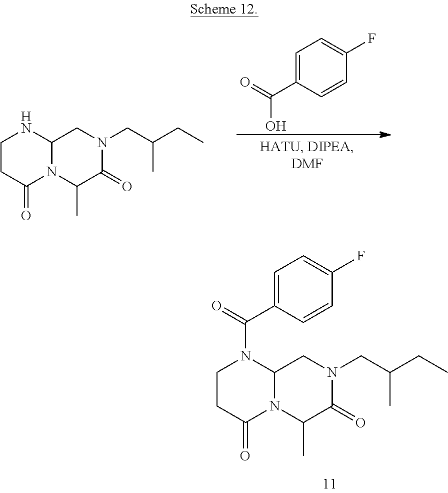 [SMILES: O=C(O)c1ccc(F)cc1 ; CCC(C)CN1CC2NCCC(=O)N2C(C)C1=O ; CCC(C)CN1CC2N(C(=O)c3ccc(F)cc3)CCC(=O)N2C(C)C1=O]

To a solution of 6-methyl-8-(2-methylbutyl)hexahydro-4H-pyrazino[1,2-a]pyrimidine-4,7(6H)-dione (250 mg, 0.98 mmol) and 4-fluorobenzoic acid (153 mg, 1.09 mmol) in DMF (4 mL) at 0° C. was added HATU (413 mg, 1.08 mmol) followed by DIPEA (0.35 mL, 1.97 mmol). The reaction mixture was stirred at room temperature for 12 h. After completion, the reaction mixture was quenched with ice cold water (50 mL) and the aqueous layer was extracted with EtOAc (50 mL×2). The organic layer was washed with cold H₂O (30 mL) followed by saturated brine (30 mL), dried over Na₂SO₄ and concentrated under reduced pressure. The crude obtained was purified by column chromatography (Silica 100-200 mesh; 30% EtOAc in hexanes) to afford 1-(4-fluorobenzoyl)-6-methyl-8-(2-methylbutyl)hexahydro-4H-pyrazino[1,2-a]pyrimidine-4,7(6H)-dione 11 (140 mg, 0.37 mmol, 38.0% yield) as a solid. MS (ESI) m/z [M+H]⁺: 376.05. ¹H NMR (400 MHz, DMSO-d₆) δ 0.69-0.81 (m, 3H) 0.86 (t, J=7.23 Hz, 3H) 0.95-1.14 (m, 2H) 1.20-1.43 (m, 4H) 1.59-1.80 (m, 2H) 2.26 (d, J=16.95 Hz, 1H) 2.55-2.72 (m, 1H) 3.20-3.31 (m, 2H) 3.35-3.39 (m, 1H) 3.52-3.70 (m, 2H) 4.73-4.89 (m, 1H) 7.33 (t, J=8.73 Hz, 2H) 7.61 (dd, J=8.23, 5.73 Hz, 2H).

### Example S13. Synthesis of Compound 12

The synthetic route for preparing Compound 12 is shown in Scheme 13.

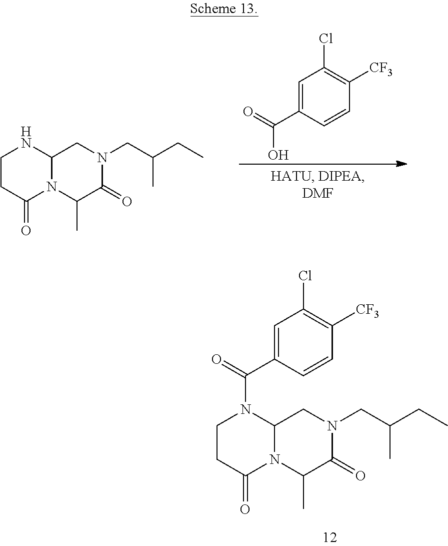 [SMILES: CCC(C)CN1CC2NCCC(=O)N2C(C)C1=O ; O=C(O)c1ccc(C(F)(F)F)c(Cl)c1 ; CCC(C)CN1CC2N(C(=O)c3ccc(C(F)(F)F)c(Cl)c3)CCC(=O)N2C(C)C1=O]

To a solution of 6-methyl-8-(2-methylbutyl)hexahydro-4H-pyrazino[1,2-a]pyrimidine-4,7(6H)-dione (250 mg, 0.98 mmol) and 3-chloro-4-(trifluoromethyl)benzoic acid (242 mg, 1.09 mmol) in DMF (4 mL) at 0° C. was added HATU (413 mg, 1.08 mmol) followed by DIPEA (0.35 mL, 1.97 mmol). The reaction mixture was stirred at room temperature for 12 h. After completion, the reaction mixture was quenched with ice cold water (50 mL) and the aqueous layer was extracted with EtOAc (50 mL×2). The organic layer was washed with cold H₂O (30 mL) followed by saturated brine (30 mL), dried over Na₂SO₄ and concentrated under reduced pressure. The crude obtained was purified by column chromatography (Silica 100-200 mesh; 30% EtOAc in hexanes) to afford 1-(3-chloro-4-(trifluoromethyl)benzoyl)-6-methyl-8-(2-methylbutyl)hexahydro-4H-pyrazino[1,2-a]pyrimidine-4,7(6H)-dione 12 (250 mg, 0.55 mmol, 55.2% yield) as a solid. MS (ESI) m/z [M+H]⁺: 460.0. ¹H NMR (400 MHz, DMSO-d₆) δ 0.74-0.93 (m, 6H) 0.98-1.19 (m, 2H) 1.28-1.46 (m, 3H) 1.64-1.81 (m, 1H) 2.22 (d, J=17.45 Hz, 1H) 2.57-2.70 (m, 1H) 3.14 (dd, J=13.21, 6.23 Hz, 1H) 3.25-3.31 (m, 2H) 3.44-3.57 (m, 1H) 3.61-3.87 (m, 2H) 4.78-4.90 (m, 1H) 5.89-6.05 (m, 1H) 7.72 (d, J=7.98 Hz, 1H) 7.90-8.02 (m, 2H).

### Example S14. Synthesis of Intermediate Compound 8-(4-methoxybenzyl)-6-methylhexahydro-4H-pyrazino[1,2-a]pyrimidine-4,7(6H)-dione

The synthetic route for preparing this intermediate compound is shown in Scheme 14.

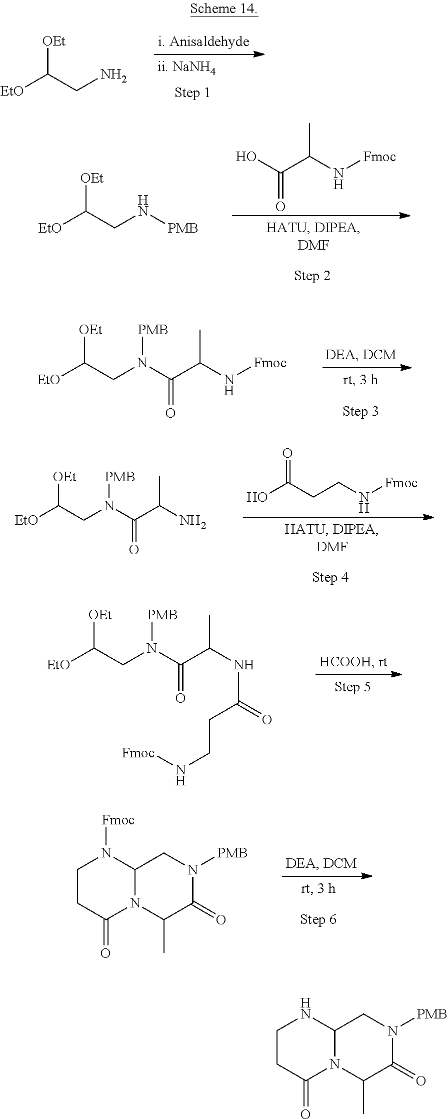

Step 1: Synthesis of 2,2-diethoxy-N-(4-methoxybenzyl)ethan-1-amine. A 500 mL round bottom flask was charged with anisaldehyde (12 mL, 90.22 mmol) and 2,2-diethoxyethanamine (10 g, 75.18 mmol). The reaction mixture was heated at 100° C. for 1 h. The reaction mixture was allowed to cool at room temperature and to this was added EtOH (100 mL) followed by NaBH₄ (4.28 g, 112.7 mmol). The resulting reaction mixture was stirred at room temperature for 16 h. After complete consumption of starting material (monitored by TLC), the reaction mixture was concentrated under reduced vacuum. The crude obtained was dissolved in EtOAc (300 mL). The organic layer was washed with brine (100 mL), dried over Na₂SO₄ and concentrated under vacuum to give crude product. The crude product obtained was purified by column chromatography (silica 100-200 mesh; 70% EtOAc in hexanes) to obtain 2,2-diethoxy-N-(4-methoxybenzyl)ethan-1-amine (15 g, 59.28 mmol, 78% yield) as a liquid. MS (ESI) m/z [M+H]⁺: 254.3.

Step 2: (9H-fluoren-9-yl)methyl (1-((2,2-diethoxyethyl)(4-methoxybenzyl)amino)-1-oxopropan-2-yl)carbamate. To a stirred solution of (((9H-fluoren-9-yl)methoxy)carbonyl)alanine (32 g, 102.76 mmol) in dry DMF (140 mL) maintained at 0° C. was added HATU (42 g, 110.67 mmol), DIPEA (21.06 mL, 118.57 mmol), followed by 2,2-diethoxy-N-(4-methoxybenzyl)ethan-1-amine (20 g, 79.05 mmol). The reaction mixture was stirred at room temperature for 16 h. After complete consumption of starting material, the reaction mixture was quenched with ice cold water (300 mL) and the aqueous layer was extracted with EtOAc (200 mL×2). The organic layer was washed with cold H₂O (200 mL) followed by brine (100 mL), dried over Na₂SO₄ and concentrated under reduced pressure to give crude product. The crude obtained was purified by column chromatography (Silica 100-200 mesh; 50% EtOAc in hexanes) to afford (9H-fluoren-9-yl)methyl (1-((2,2-diethoxyethyl)(4-methoxybenzyl)amino)-1-oxopropan-2-yl)carbamate (28 g, 51.22 mmol, 64.8% yield) as a gummy liquid. MS (ESI) m/z [M+H-EtOH]⁺: 501.2.

Step 3: Synthesis of 2-amino-N-(2,2-diethoxyethyl)-N-(4-methoxybenzyl)propanamide. To a solution of (9H-fluoren-9-yl) methyl (1-((2,2-diethoxyethyl)(4-methoxybenzyl)amino)-1-oxopropan-2-yl)carbamate (28 g, 51.22 mmol) in CH₂C₁₂ (30 mL) was added diethylamine (200 mL). The reaction mixture was stirred at room temperature for 3 h. After complete consumption of the starting material (monitored by TLC), the reaction mixture was concentrated and the crude obtained was purified by column chromatography (Silica 100-200 mesh; 5% MeOH in DCM) to afford 2-amino-N-(2,2-diethoxyethyl)-N-(4-methoxybenzyl)propanamide (14.5 g, 44.75 mmol, 87% yield) as a viscous liquid. MS (ESI) m/z [M+H-EtOH]⁺: 279.05.

Step 4: Synthesis of (9H-fluoren-9-yl)methyl (3-((1-((2,2-diethoxyethyl)(4-methoxybenzyl)amino)-1-oxopropan-2-yl)amino)-3-oxopropyl)carbamate. To a stirred solution of 3-((((9H-fluoren-9-yl)methoxy)carbonyl)amino)propanoic acid (14.78 g, 47.53 mmol) in dry DMF (120 mL) maintained at 0° C. was added HATU (18.06 g, 47.53 mmol), DIPEA (9.21 mL, 51.85 mmol) followed by 2-amino-N-(2,2-diethoxyethyl)-N-(4-methoxybenzyl)propanamide (14 g, 43.20 mmol). The reaction mixture was stirred for 16 h at room temperature. After completion, the reaction mixture was quenched with ice cold water (200 mL) and the aqueous layer was extracted with EtOAc (200 mL×2). The organic layer was washed with cold H₂O (500 mL) followed by saturated brine (200 mL), dried over Na₂SO₄ and concentrated under reduced pressure. The crude obtained was purified by column chromatography (Silica 100-200 mesh; 30% EtOAc in hexanes) to afford (9H-fluoren-9-yl)methyl (3-((1-((2,2-diethoxyethyl)(4-methoxybenzyl)amino)-1-oxopropan-2-yl)amino)-3-oxopropyl)carbamate (18 g, 29.14 mmol, 67.44% yield) as a viscous liquid. MS (ESI) m/z [M+H-EtOH]⁺: 572.

Step 5: Synthesis of (9H-fluoren-9-yl)methyl 8-(4-methoxybenzyl)-6-methyl-4,7-dioxohexahydro-2H-pyrazino[1,2-a]pyrimidine-1(6H)-carboxylate. A solution of (9H-fluoren-9-yl)methyl (3-((1-((2,2-diethoxyethyl)(4-methoxybenzyl)amino)-1-oxopropan-2-yl)amino)-3-oxopropyl)carbamate (18 g, 29.14 mmol) in formic acid (120 mL) was stirred at room temperature for 12 h. After completion, the reaction mixture was concentrated and the crude obtained was purified by column chromatography (Silica 100-200 mesh; 30% EtOAc in hexanes) to afford (9H-fluoren-9-yl)methyl 8-(4-methoxybenzyl)-6-methyl-4,7-dioxohexahydro-2H-pyrazino[1,2-a]pyrimidine-1(6H)-carboxylate (14.5 g, 27.58 mmol, 94% yield) as a solid. MS (ESI) m/z [M+H]⁺: 526.

Step 6: Synthesis of 8-(4-methoxybenzyl)-6-methylhexahydro-4H-pyrazino[1,2-a]pyrimidine-4,7(6H)-dione. To a solution of (9H-fluoren-9-yl)methyl 8-(4-methoxybenzyl)-6-methyl-4,7-dioxohexahydro-2H-pyrazino[1,2-a]pyrimidine-1(6H)-carboxylate (14 g, 26.63 mmol) in CH₂Cl₂ (150 mL) was added diethyl amine (100 mL) and the reaction mixture was stirred at room temperature for 3 h. After complete consumption of the starting material (monitored by TLC), the reaction mixture was concentrated and the crude obtained was purified by column chromatography (Silica 100-200 mesh; 5% MeOH in DCM) to afford 8-(4-methoxybenzyl)-6-methylhexahydro-4H-pyrazino[1,2-a]pyrimidine-4,7(6H)-dione (7 g, 23.07 mmol, 87% yield) as a sticky solid. MS (ESI) m/z [M+H]⁺: 304.

### Example S15. Synthesis of Intermediate Compound 8-(4-methoxybenzyl)-6-methylhexahydro-4H-pyrazino[1,2-a]pyrimidine-4,7(6H)-dione

The synthetic route for preparing this intermediate compound is shown in Scheme 15.

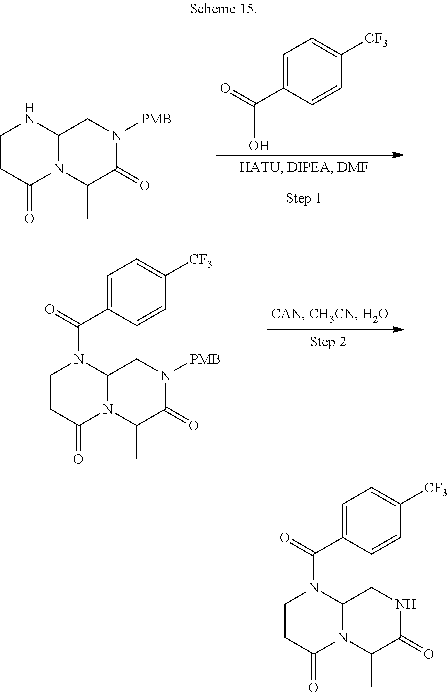

Step 1: Synthesis of 8-(4-methoxybenzyl)-6-methyl-1-(4-(trifluoromethyl)benzoyl)hexahydro-4H-pyrazino[1,2-a]pyrimidine-4,7(6H)-dione. To a solution of 4-(trifluoromethyl)benzoic acid (5.26 g, 27.69 mmol) in DMF (100 mL) maintained at 0° C. was added HATU (10.52 g, 27.69 mmol), DIPEA (12.30 mL, 69.23 mmol) followed by 8-(4-methoxybenzyl)-6-methylhexahydro-4H-pyrazino[1,2-a]pyrimidine-4,7(6H)-dione (7 g, 23.07 mmol), and the reaction mixture was stirred at room temperature for 12 h. After completion, the reaction mixture was quenched with ice cold water (200 mL) and the aqueous layer was extracted with EtOAc (200 mL×2). The organic layer was washed with cold H₂O (200 mL) followed by saturated brine (150 mL), dried over Na₂SO₄ and concentrated under reduced pressure. The crude obtained was purified by column chromatography (Silica 100-200 mesh; 30% EtOAc in hexanes) to afford 8-(4-methoxybenzyl)-6-methyl-1-(4-(trifluoromethyl)benzoyl)hexahydro-4H-pyrazino[1,2-a]pyrimidine-4,7(6H)-dione (9 g, 18.92 mmol, 82.04% yield) as a solid. MS (ESI) m/z [M+H]⁺: 476.15 and MS (ESI) m/z [M+Na]⁺: 498.05.

Step 2: Synthesis of 6-methyl-1-(4-(trifluoromethyl)benzoyl)hexahydro-4H-pyrazino[1,2-a]pyrimidine-4,7(6H)-dione. To a solution of 8-(4-methoxybenzyl)-6-methyl-1-(4-(trifluoromethyl)benzoyl)hexahydro-4H-pyrazino[1,2-a]pyrimidine-4,7(6H)-dione (9 g, 18.92 mmol) in CH₃CN:H₂O (2:1, 150 mL) maintained at 0° C., was added CAN (31.15 g, 56.82 mmol) and the reaction mixture was allowed to stir at room temperature for 3 h. Progress of the reaction was monitored by TLC. After completion, the reaction mixture was quenched with saturated solution of aq. NaHCO₃ (200 mL) and extracted with EtOAc (200 mL×2). Combined organic layer was washed with H₂O (200 mL) followed by saturated brine solution (150 mL), dried over Na₂SO₄ and concentrated under reduced pressure. The crude obtained was purified by column chromatography (Silica 100-200 mesh; 10% MeOH in DCM) to afford 6-methyl-1-(4-(trifluoromethyl)benzoyl)hexahydro-4H-pyrazino[1,2-a]pyrimidine-4,7(6H)-dione (3.5 g, 9.85 mmol, 52.8% yield) as a solid. MS (ESI) m/z [M+H+CH₃CN]⁺: 397.0. ¹H NMR (400 MHz, DMSO-d₆) δ 1.25-1.46 (m, 3H) 2.15-2.30 (m, 1H) 2.56-2.69 (m, 1H) 3.16 (d, J=4.99 Hz, 1H) 3.22-3.30 (m, 1H) 3.42-3.72 (m, 2H) 4.70-4.87 (m, 1H) 5.85-5.95 (m, 1H) 7.75 (d, J=7.98 Hz, 2H) 7.86 (d, J=7.98 Hz, 2H) 8.11 (brs, 1H).

### Example S16. General Procedure A for the Synthesis of Final Compounds

To a solution of 6-methyl-1-(4-(trifluoromethyl)benzoyl)hexahydro-4H-pyrazino[1,2-a]pyrimidine-4,7(6H)-dione (200 mg, 0.56 mmol) in DMF (2 mL) was added KO^{t}Bu (1M in THF, 1.69 mmol, 1.69 mL) followed by alkyl halide (1.12 mmol), and the reaction mixture was exposed to microwave irradiation at 120° C. for 1 h. The reaction mixture was cooled to room temperature and quenched with H₂O (25 mL). The aqueous layer was extracted with EtOAc (10 mL×3). The combined organic layers were washed with brine and concentrated. The crude product obtained was purified by CombiFlash.

### Example S17. Synthesis of Compound 15

Compound 15 was synthesized by General Procedure A using (bromomethyl)cyclopentane as the alkyl halide. MS (ESI) m/z [M+H]⁺: 438.65. ¹H NMR (400 MHz, DMSO-d₆) δ 1.02-1.26 (m, 3H) 1.28-1.42 (m, 2H) 1.44-1.76 (m, 6H) 1.80-2.08-2.33 (m, 2H) 2.55-2.71 (m, 1H) 3.22 (dd, J=12.96, 7.48 Hz, 1H) 3.26-3.32 (m, 1H) 3.39 (d, J=6.98 Hz, 1H) 3.49-3.57 (m, 1H) 3.59-3.74 (m, 1H) 3.76-3.91 (m, 1H) 4.80-4.90 (m, 1H) 5.95-6.05 (m, 1H) 7.72-7.79 (m, 2H) 7.84-7.91 (m, 2H).

### Example S18. Synthesis of Compound 16

Compound 16 was synthesized by General Procedure A using bromomethylcyclobutane as the alkyl halide. MS (ESI) m/z [M+H]⁺: 424.15. ¹H NMR (400 MHz, DMSO-d₆) δ 1.29-1.44 (m, 2H) 1.58-1.89 (m, 4H) 1.90-2.08 (m, 2H) 2.16-2.31 (m, 1H) 2.55-2.70 (m, 2H) 3.18-3.31 (m, 1H) 3.25-3.26 (m, 1H) 3.34-3.42 (m, 1H) 3.36-3.57 (m, 2H) 3.60-3.69 (m, 1H) 3.71-3.83 (m, 1H) 4.75-4.89 (m, 1H) 5.90-6.05 (m, 1H) 7.70-7.79 (m, 2H) 7.87 (d, J=8.31 Hz, 2H).

### Example S19. Synthesis of Compound 19

Compound 19 was synthesized by General Procedure A using (2-bromoethyl)cyclopentane as the alkyl halide. MS (ESI) m/z [M+H]⁺: 452.35. ¹H NMR (400 MHz, DMSO-d₆) δ 0.94-1.18 (m, 3H) 1.26-1.61 (m, 9H) 1.66-1.83 (m, 2H) 2.16-2.31 (m, 1H) 2.56-2.70 (m, 1H) 3.16-3.28 (m, 1H) 3.35-3.56 (m, 3H) 3.60-3.73 (m, 1H) 3.77-3.90 (m, 1H) 4.72-4.92 (m, 1H) 5.94-6.06 (m, 1H) 7.77 (d, J=7.98 Hz, 2H) 7.87 (d, J=7.98 Hz, 2H).

### Example S20. Synthesis of Compound 20

Compound 20 was synthesized by General Procedure A using (2-bromoethyl)cyclobutane as the alkyl halide. MS (ESI) m/z [M+H]⁺: 438.25. ¹H NMR (400 MHz, DMSO-d₆) δ 1.27-1.44 (m, 3H) 1.50-1.71 (m, 4H) 1.71-1.88 (m, 2H) 1.93-2.09 (m, 2H) 2.13-2.34 (m, 2H) 2.56-2.70 (m, 2H) 3.25-3.32 (m, 1H) 3.35-3.42 (m, 1H) 3.45-3.55 (m, 1H) 3.59-3.72 (m, 1H) 3.74-3.90 (m, 1H) 4.75-4.89 (m, 1H) 5.94-6.05 (m, 1H) 7.71-7.79 (m, 2H) 7.87 (d, J=8.31 Hz, 2H).

### Example S21. Synthesis of Compound 21

Compound 21 was synthesized by General Procedure A using 1-bromobutane as the alkyl halide. MS (ESI) m/z [M+H]⁺: 412.20. ¹H NMR (400 MHz, DMSO-d₆) δ 0.81-0.97 (m, 3H) 1.15-1.57 (m, 7H) 2.15-2.31 (m, 1H) 2.57-2.69 (m, 1H) 3.14-3.28 (m, 1H) 3.35-3.60 (m, 3H) 3.62-3.73 (m, 1H) 3.74-3.92 (m, 1H) 4.75-4.91 (m, 1H) 5.94-6.06 (m, 1H) 7.76 (d, J=7.34 Hz, 2H) 7.87 (d, J=7.83 Hz, 2H).

### Example S22. Synthesis of Compound 22

Compound 22 was synthesized by General Procedure A using 4-bromobut-1-ene as the alkyl halide. MS (ESI) m/z [M+H]⁺: 410.20. ¹H NMR (400 MHz, DMSO-d₆) δ 1.28-1.45 (m, 3H) 2.14-2.38 (m, 3H) 2.55-2.69 (m, 1H) 3.36-3.57 (m, 4H) 3.58-3.72 (m, 1H) 3.75-3.89 (m, 1H) 4.75-4.90 (m, 1H) 4.98-5.19 (m, 2H) 5.69-5.84 (m, 1H) 5.93-6.05 (m, 1H) 7.76 (d, J=7.98 Hz, 2H) 7.88 (d, J=7.98 Hz, 2H).

### Example S23. Synthesis of Compound 23

Compound 23 was synthesized by General Procedure A using 1-bromo-2-methylpropane as the alkyl halide. MS (ESI) m/z [M+H]⁺: 412.25. ¹H NMR (400 MHz, DMSO-d₆) δ 0.80-0.96 (m, 6H) 1.30-1.48 (m, 3H) 1.85-2.03 (m, 1H) 2.15-2.31 (m, 1H) 2.57-2.70 (m, 1H) 3.06-3.16 (m, 1H) 3.18-3.28 (m, 1H) 3.36-3.45 (m, 1H) 3.44-3.57 (m, 1H) 3.60-3.74 (m, 1H) 3.73-3.87 (m, 1H) 4.77-4.92 (m, 1H) 5.93-6.07 (m, 1H) 7.76 (d, J=7.48 Hz, 2H) 7.87 (d, J=7.48 Hz, 2H).

### Example S24. Synthesis of Compound 24

Compound 24 was synthesized by General Procedure A using 2-bromopropane as the alkyl halide. MS (ESI) m/z [M+H]⁺: 398.55. ¹H NMR (400 MHz, DMSO-d₆) δ 1.10 (d, J=5.49 Hz, 6H) 1.28-1.45 (m, 3H) 2.16-2.24 (m, 1H) 2.56-2.71 (m, 1H) 3.34-3.40 (m, 1H) 3.44-3.79 (m, 3H) 4.59-4.72 (m, 1H) 4.75-4.90 (m, 1H) 5.86-6.00 (m, 1H) 7.79 (d, J=7.98 Hz, 2H) 7.83-7.92 (m, 2H).

### Example S25. Synthesis of Intermediate Compound 1-(4-(difluoromethoxy)benzoyl)-6-methylhexahydro-4H-pyrazino[1,2-a]pyrimidine-4,7(6H)-dione

The synthetic route for preparing this intermediate compound is shown in Scheme 16.

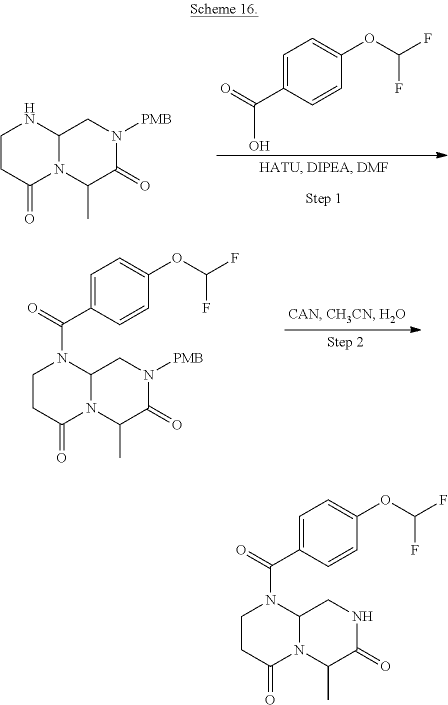

Step 1: Synthesis of 1-(4-(difluoromethoxy)benzoyl)-8-(4-methoxybenzyl)-6-methylhexahydro-4H-pyrazino[1,2-a]pyrimidine-4,7(6H)-dione. To a solution of 4-(difluoromethoxy)benzoic acid (1.71 g, 9.08 mmol) in DMF (25 mL) maintained at 0° C. was added HATU (3.45 g, 9.08 mmol), DIPEA (4.34 mL, 24.8 mmol) followed by 8-(4-methoxybenzyl)-6-methylhexahydro-4H-pyrazino[1,2-a]pyrimidine-4,7(6H)-dione (2.5 g, 8.25 mmol) and reaction mixture was stirred at room temperature for 12 h. After completion, the reaction mixture was quenched with ice cold water (50 mL) and the aqueous layer was extracted with EtOAc (100 mL×2). The organic layer was washed with cold H₂O (100 mL) followed by saturated brine (100 mL), dried over Na₂SO₄ and concentrated under reduced pressure. The crude obtained was purified by column chromatography (Silica 100-200 mesh; 30% EtOAc in hexanes) to afford 1-(4-(difluoromethoxy)benzoyl)-8-(4-methoxybenzyl)-6-methylhexahydro-4H-pyrazino[1,2-a]pyrimidine-4,7(6H)-dione (3.5 g, 7.38 mmol, 89.5% yield) as a solid. MS (ESI) m/z [M+H]⁺: 474.12.

Step 2: Synthesis of 1-(4-(difluoromethoxy)benzoyl)-6-methylhexahydro-4H-pyrazino[1,2-a]pyrimidine-4,7(6H)-dione. To a solution of 1-(4-(difluoromethoxy)benzoyl)-8-(4-methoxybenzyl)-6-methylhexahydro-4H-pyrazino[1,2-a]pyrimidine-4,7(6H)-dione (3.0 g, 6.34 mmol) in CH₃CN:H₂O (2:1, 45 mL) maintained at 0° C., was added CAN (12.0 g, 21.90 mmol) and the reaction mixture was allowed to stir at room temperature for 3 h. Progress of the reaction was monitored by TLC. After completion, the reaction mixture was quenched with saturated solution of aq. NaHCO₃ (100 mL) and extracted with EtOAc (200 mL×2). The combined organic layer was washed with H₂O (250 mL) followed by saturated brine solution (250 mL), dried over Na₂SO₄ and concentrated under reduced pressure. The crude obtained was purified by column chromatography (Silica 100-200 mesh; 10% MeOH in DCM) to afford 1-(4-(difluoromethoxy)benzoyl)-6-methylhexahydro-4H-pyrazino[1,2-a]pyrimidine-4,7(6H)-dione (2.0 g, 5.66 mmol, 89.6% yield) as a solid. MS (ESI) m/z [M+H]⁺: 353.95. ¹H NMR (400 MHz, DMSO-d₆) δ 1.10-1.39 (m, 3H) 2.17-2.18 (m, 1H) 2.52-2.68 (m, 1H) 3.18-3.27 (m, 2H) 3.44-3.71 (m, 2H) 4.69-4.83 (m, 1H) 5.75-5.92 (m, 1H) 7.24 (d, J=7.83 Hz, 2H) 7.32 (t, J=72.0 Hz, 1H) 7.57 (d, J=8.31 Hz, 2H) 8.04 (brs, 1H).

### Example S26. General Procedure B for the Synthesis of Final Compounds

To a solution of 1-(4-(difluoromethoxy)benzoyl)-6-methylhexahydro-4H-pyrazino[1,2-a]pyrimidine-4,7(6H)-dione (200 mg, 0.56 mmol) in DMF (4 mL) maintained at 0° C. was added NaH (122 mg, 2.8 mmol, 55% dispersion in mineral oil) and the reaction mixture was stirred at the same temperature for 15 minutes. To this reaction mixture was added alkyl halide (1.6 mmol) and the reaction mixture was allowed to warm to room temperature and stirred for 3 h. After completion, the reaction mixture was quenched with ice cold H₂O (15 mL) and aqueous layer was extracted with EtOAc (15 mL×3). The combined organic layer was washed with brine and concentrated. The crude product obtained was purified by CombiFlash.

### Example S27. Synthesis of Compound 13

Compound 13 was synthesized by General Procedure B using (bromomethyl)cyclopentane as the alkyl halide. MS (ESI) m/z [M+H]⁺: 436.05. ¹H NMR (400 MHz, DMSO-d₆) δ 1.07-1.16 (m, 3H) 1.32 (d, J=6.48 Hz, 3H) 1.41-1.73 (m, 7H) 2.06-2.21 (m, 1H) 2.21-2.34 (m, 1H) 2.54-2.70 (m, 1H) 3.14-3.29 (m, 1H) 3.35-3.45 (m, 1H) 3.52-3.69 (m, 1H) 3.75-3.93 (m, 1H) 4.75-4.91 (m, 1H) 5.88-5.99 (m, 1H) 7.27 (d, J=8.48 Hz, 2H) 7.35 (t, J=72.0 Hz, 1H) 7.61 (d, J=8.98 Hz, 2H).

### Example S28. Synthesis of Compound 14

Compound 14 was synthesized by General Procedure B using (bromomethyl)cyclobutane as the alkyl halide. MS (ESI) m/z [M+H]⁺: 421.14. ¹H NMR (400 MHz, DMSO-d₆) δ 1.16-1.25 (m, 1H) 1.27-1.43 (m, 3H) 1.54-1.73 (m, 2H) 1.73-1.86 (m, 2H) 1.89-2.03 (m, 2H) 2.24 (d, J=17.12 Hz, 1H) 2.53-2.69 (m, 2H) 3.20-3.28 (m, 1H) 3.29-3.40 (m, 1H) 3.40-3.66 (m, 2H) 3.69-3.87 (m, 1H) 4.75-4.86 (m, 1H) 5.74-6.02 (m, 1H) 7.26 (d, J=8.31 Hz, 2H)) 7.33 (t, J=72.0 Hz, 1H) 7.59 (d, J=8.31 Hz, 2H).

### Example S29. Synthesis of Compound 17

Compound 20 was synthesized by General Procedure B using 1-bromobutane as the alkyl halide. MS (ESI) m/z [M+H]⁺: 410.0. ¹H NMR (400 MHz, DMSO-d₆) δ 0.81-0.96 (m, 3H) 1.15-1.39 (m, 4H) 1.40-1.55 (m, 2H) 2.26 (d, J=16.95 Hz, 1H) 2.53-2.70 (m, 2H) 3.12-3.30 (m, 2H) 3.38-3.46 (m, 1H) 3.56-3.74 (m, 2H) 3.75-3.92 (m, 1H) 4.84 (q, J=6.81 Hz, 1H) 5.86-6.06 (m, 1H) 7.28 (d, J=7.98 Hz, 2H) 7.36 (t, J=72.0 Hz, 1H) 7.62 (d, J=8.48 Hz, 2H).

### Example S30. Synthesis of Compound 18

Compound 18 was synthesized by General Procedure B using 4-bromobut-1-ene as the alkyl halide. MS (ESI) m/z [M+H]⁺: 408.06. ¹H NMR (400 MHz, DMSO-d₆) δ 1.16-1.45 (m, 3H) 2.18-2.33 (m, 3H) 2.53-2.70 (m, 1H) 3.36-3.46 (m, 3H) 3.51-3.72 (m, 2H) 3.74-3.90 (m, 1H) 4.84 (q, J=6.65 Hz, 1H) 4.91-5.15 (m, 2H) 5.67-5.84 (m, 1H) 5.86-6.03 (m, 1H) 7.29 (d, J=8.48 Hz, 2H) 7.36 (t, J=72.0 Hz, 1H) 7.61 (d, J=8.48 Hz, 2H).

### Example S31. Synthesis of Compound 27

Compound 27 was synthesized by General Procedure B using 2-(bromomethyl)tetrahydrofuran as the alkyl halide. MS (ESI) m/z [M+H]⁺: 438.1. ¹H NMR (400 MHz, CDCl₃) δ 7.48-7.55 (m, 2H), 7.20-7.30 (m, 2H), 6.40-6.76 (m, 1H), 5.90-6.20 (m, 1H), 5.16-5.26 (m, 1H), 4.06-4.17 (m, 2H), 3.82-3.92 (m, 4H), 3.61-3.77 (m, 2H), 2.83-2.99 (m, 1H), 2.47-2.59 (m, 2H), 2.01-2.12 (m, 4H), 1.49 (s, 3H).

### Example S32. Synthesis of Compound 28

Compound 28 was synthesized by General Procedure B using (2-bromoethyl)benzene as the alkyl halide. MS (ESI) m/z [M+H]⁺: 458.10. ¹H NMR (400 MHz, CDCl₃) δ 7.40-7.50 (m, 2H), 7.20-7.28 (m, 2H), 7.33-7.43 (m, 5H), 6.40-6.76 (m, 1H), 5.90-6.20 (m, 1H), 5.16-5.26 (m, 1H), 3.72-3.96 (m, 2H), 3.44-3.52 (m, 1H), 3.25-3.35 (m, 2H), 2.83-2.99 (m, 2H), 2.47-2.59 (m, 1H), 2.42-2.60 (m, 1H), 2.30-2.57 (m, 1H), 1.49 (s, 3H).

### Example S33. Synthesis of Compound 29

Compound 29 was synthesized by General Procedure B using 4-(2-bromoethyl)pyridine as the alkyl halide. MS (ESI) m/z [M+H]⁺: 459.10. ¹H NMR (400 MHz, CDCl₃) δ 8.50-8.58 (m, 2H), 7.24-7.46 (m, 4H), 7.18 (d, J=7.99 Hz, 2H), 6.40-6.76 (m, 1H), 5.90-6.20 (m, 1H), 5.16-5.26 (m, 1H), 3.72-3.96 (m, 2H), 3.44-3.52 (m, 1H), 3.25-3.35 (m, 2H), 2.83-2.99 (m, 2H), 2.47-2.59 (m, 1H), 2.42-2.60 (m, 1H), 2.30-2.57 (m, 1H), 1.49 (s, 3H).

### Example S34. Synthesis of Compound 30

Compound 30 was synthesized by General Procedure B using (3-bromopropyl)cyclopropane as the alkyl halide. MS (ESI) m/z [M+H]⁺: 459.10. ¹H NMR (400 MHz, CDCl₃) δ 8.50-8.58 (m, 2H), 7.24-7.46 (m, 4H), 7.18 (d, J=7.99 Hz, 2H), 6.40-6.76 (m, 1H), 5.90-6.20 (m, 1H), 5.16-5.26 (m, 1H), 3.72-3.96 (m, 2H), 3.44-3.52 (m, 1 H), 3.25-3.35 (m, 2H), 2.83-2.99 (m, 2H), 2.47-2.59 (m, 1H), 2.42-2.60 (m, 1H), 2.30-2.57 (m, 1H), 1.49 (s, 3H).

### Example S35. Synthesis of Compound 31

Compound 31 was synthesized by General Procedure B using (2-bromoethyl)cyclopropane as the alkyl halide. MS (ESI) m/z [M+H]⁺: 422.2. ¹H NMR (400 MHz, CDCl₃) δ 7.48 (d, J=8.01 Hz, 2H), 7.20-7.28 (m, 2H), 6.40-6.76 (m, 1H), 5.90-6.20 (m, 1H), 5.16-5.26 (m, 1H), 3.72-3.96 (m, 1H), 3.46-3.64 (m, 5H), 2.46-2.64 (m, 2H), 1.43-1.56 (m, 5H), 0.43-0.65 (m, 2H), 0.75-0.85 (m, 2H).

### Example S36. Synthesis of Compound 32

Compound 32 was synthesized by General Procedure B using 1-bromo-2-methoxyethane as the alkyl halide. MS (ESI) m/z [M+H]⁺: 412.1. ¹H NMR (400 MHz, DMSO-d₆) δ 7.52-7.62 (m, 2H), 7.16-7.34 (m, 3H), 5.85-5.95 (m, 1H), 4.80-4.90 (m, 1H), 3.85-3.95 (m, 1H), 3.70-3.80 (m, 2H), 3.25-3.46 (m, 5H), 3.22 (s, 3H), 2.62-2.72 (m, 1H), 2.20-2.30 (m, 1H), 1.49 (s, 3H).

### Example S37. Synthesis of Compound 33

Compound 33 was synthesized by General Procedure B using 1-bromo-3-methoxypropane as the alkyl halide. MS (ESI) m/z [M+H]⁺: 426.20. ¹H NMR (400 MHz, DMSO-d₆) δ 7.52-7.62 (m, 2H), 7.16-7.34 (m, 3H), 5.85-5.95 (m, 1H), 4.80-4.90 (m, 1H), 3.85-3.95 (m, 1H), 3.70-3.80 (m, 2H), 3.58-3.68 (m, 2H), 3.45-3.55 (m, 4H), 3.22 (s, 3H), 2.62-2.72 (m, 1H), 2.20-2.30 (m, 2H), 1.49 (s, 3H).

### Example S38. Synthesis of Compound 36

Compound 36 was synthesized by General Procedure B using (2-bromoethyl)methylsulfone as the alkyl halide. MS (ESI) m/z [M+H]⁺: 459.95. ¹H NMR (400 MHz, CHLOROFORM) δ 7.49 (d, J=8.01 Hz, 2H), 7.15-7.26 (m, 2H), 6.40-6.76 (m, 1H), 5.90-6.20 (m, 1H), 5.15-5.25 (m, 1H), 3.86-3.97 (m, 3H), 3.66-3.77 (m, 2H), 3.38-3.49 (m, 3H), 2.97 (s, 3H), 2.59-2.69 (m, 2H), 1.49 (s, 3H).

### Example S39. Synthesis of Compound 34

Step 1. To a solution of 1-(4-(difluoromethoxy)benzoyl)-6-methylhexahydro-4H-pyrazino[1,2-a]pyrimidine-4,7(6H)-dione (0.300 g, 0.849 mmol) in DMF (6 mL) was added Cs₂CO₃ (0.827 g, 2.547 mmol) followed by (2-bromoethoxy)(tert-butyl)dimethylsilane (0.243 g, 1.018 mmol) at 0° C. and the reaction mixture was heated at 120° C. in sealed tube for 1 h. Progress of the reaction was monitored by TLC. After completion, the reaction mixture was slowly quenched with ice cold water (30 mL) and extracted with EtOAc (50 mL). Combined organic layer was washed with ice cold brine solution (3×30 mL), dried over Na₂SO₄ and concentrated under reduced pressure to afford 8-(2-((tert-butyldimethylsilyl)oxy)ethyl)-1-(4-(difluoromethoxy)benzoyl)-6-methylhexahydro-4H-pyrazino[1,2-a]pyrimidine-4,7(6H)-dione (0.250 g, crude). The crude compound was as such used for next reaction without carried out further purification. MS (ESI) m/z [M+H]⁺: 512.10.

Step 2. To a solution of 8-(2-((tert-butyldimethylsilyl)oxy)ethyl)-1-(4-(difluoromethoxy)benzoyl)-6-methylhexahydro-4H-pyrazino[1,2-a]pyrimidine-4,7(6H)-dione (0.250 g, 0.4886 mmol) in THE (5 mL) was added TBAF (3 mL) 0° C. temperature. The reaction mixture was allowed to attain room temperature and stirred for 6 h. Progress of the reaction was monitored by TLC. After completion, the reaction mixture was slowly quenched with ice cold water (5 mL) and extracted with EtOAc (2×10 mL). Combined organic layer was washed with ice cold brine solution (10 mL), dried over Na₂SO₄ and concentrated under reduced pressure to get crude compound. The crude compound obtained was purified by column chromatography (Silicagel 60-120 mesh; 10% MeOH in DCM) to afford 1-(4-(difluoromethoxy)benzoyl)-8-(2-hydroxyethyl)-6-methylhexahydro-4H-pyrazino[1,2-a]pyrimidine-4,7(6H)-dione (0.102 g, 52% yield) white solid. MS (ESI) m/z [M+H]⁺: 398.2. ¹H NMR (400 MHz, DMSO-d₆) δ 7.52-7.62 (m, 2H), 7.16-7.34 (m, 3H), 5.92-6.02 (m, 1H), 6.78-6.88 (m, 2H), 3.86-3.92 (m, 1H), 3.47-3.62 (m, 6H), 3.21-3.31 (m, 1H), 2.57-2.67 (m, 1H), 2.25-3.35 (m, 1H), 1.49 (s, 3H).

### Example S40. Synthesis of Compound 35

Step 1. To a solution of 1-(4-(difluoromethoxy)benzoyl)-6-methylhexahydro-4H-pyrazino[1,2-a]pyrimidine-4,7(6H)-dione (0.300 g, 0.849 mmol) in DMF (6 mL) was added NaH (0.050 g, 1.274 mmol) followed by 2-bromoacetonitrile (0.112 g, 0.933 mmol) at 0° C. and the reaction mixture was allowed to stand for room temperature for 1 h. Progress of the reaction was monitored by TLC. After completion, the reaction mixture was slowly quenched with ice cold water (70 mL) and extracted with EtOAc (100 mL). Combined organic layer was washed with ice cold brine solution (100 mL), dried over Na₂SO₄ and concentrated under reduced pressure to get crude compound. The crude compound obtained was purified by column chromatography (Silicagel 60-120 mesh; 10% MeOH in DCM) to afford 2-(1-(4-(difluoromethoxy)benzoyl)-6-methyl-4,7-dioxooctahydro-8H-pyrazino[1,2-a]pyrimidin-8-yl)acetonitrile (0.120 g, 36% yield) white solid. MS (ESI) m/z [M+H]⁺: 393.05.

Step 2. To a solution of 2-(1-(4-(difluoromethoxy)benzoyl)-6-methyl-4,7-dioxooctahydro-8H-pyrazino[1,2-a]pyrimidin-8-yl)acetonitrile (0.120 g, 0.305 mmol) in ethanol (5 mL) was added Conc. HCl (0.100 mL) followed by Platinum oxide (0.012 g, 0.030 mmol) at room temperature and the reaction mixture was heated under Hydrogen gas atmosphere for 3 h. Progress of the reaction was monitored by TLC. After completion, the reaction mixture was filtered through a pad of Celite. The Celite pad was washed with ethanol (20 mL) and filtrate was concentrated under reduced pressure to get crude compound. The crude compound was triturated with n pentane to afford 8-(2-aminoethyl)-1-(4-(difluoromethoxy)benzoyl)-6-methylhexahydro-4H-pyrazino[1,2-a]pyrimidine-4,7(6H)-dione (0.110 g, 90%) yield) white solid. MS (ESI) m/z [M+H]⁺: 397.05. ¹H NMR (400 MHz, DMSO d₆) δ 7.96 (s, 2H), 7.55-7.65 (m, 2H), 7.20-7.35 (m, 3H), 5.90-6.20 (m, 1H), 4.85-4.95 (m, 1H), 3.82-3.92 (m, 1H), 3.55.-3.85 (m, 2H), 3.35-3.45 (m, 3H), 2.95-3.05 (m, 2H), 2.60-2.70 (m, 1H), 2.20-2.30 (m, 1H), 1.35 (s, 3H).

### Example S41. General Procedure C for the Synthesis of Final Compounds

To a solution of 1-(4-(difluoromethoxy)benzoyl)-6-methylhexahydro-4H-pyrazino[1,2-a]pyrimidine-4,7(6H)-dione (0.200 g, 0.566 mmol) in DMF (5 mL) was added Cs₂CO₃ (0.735 g, 2.264 mmol, 4 eq) followed by alkyl halide (0.679 mmol, 1.2 eq) at 0° C. and the reaction mixture was heated at 50° C. under microwave irradiation for 1 h. Progress of the reaction was monitored by TLC. After completion, the reaction mixture was slowly quenched with ice cold water (6 mL) and extracted with EtOAc (20 mL×3). The combined organic layer was washed with saturated brine solution (10 mL), dried over Na₂SO₄ and concentrated under reduced pressure to get crude compound. The crude compound obtained was purified by column chromatography to provide the final compound.

### Example S42. Synthesis of Compound 25

Compound 25 was synthesized by General Procedure C using 2-(2-iodoethyl)furan as the alkyl halide. MS (ESI) m/z [M+H]⁺: 448.10. ¹H NMR: δ 7.40-7.50 (m, 2H), 7.28-7.38 (m, 1H), 7.15-7.25 (m, 2H), 6.39-6.78 (m, 1H), 6.25-6.35 (m, 1H), 5.90-6.12 (m, 2H), 5.25-5.35 (m, 1H), 5.10-5.20 (m, 1H), 3.70-3.80 (m, 1H), 3.50-3.60 (m, 1H), 3.20-3.40 (m, 2H), 2.95-3.05 (m, 3H), 2.45-2.60 (m, 2H), 1.59 (s, 3H).

### Example S43. Synthesis of Compound 26

Compound 26 was synthesized by General Procedure C using 2-(2-bromoethyl)thiophene as the alkyl halide. MS (ESI) m/z [M+H]⁺: 464.1. ¹H NMR (400 MHz, CDCl₃) δ 7.40-7.48 (m, 2H), 7.15-7.26 (m, 3H), 6.85-6.95 (m, 2H), 6.39-6.95 (m, 2H), 5.90-6.20 (m, 1H), 5.15-5.25 (m, 1H), 3.72-3.96 (m, 2H), 3.47-3.54 (m, 1H), 3.32-3.42 (m, 3H), 3.10-3.20 (m, 2H), 2.42-2.56 (m, 2H), 1.49 (s, 3H).

### Example S44. Synthesis of Intermediate Compound 1-(4-(difluoromethoxy)benzyl)-6-methylhexahydro-4H-pyrazino[1,2-a]pyrimidine-4,7(6H)-dione

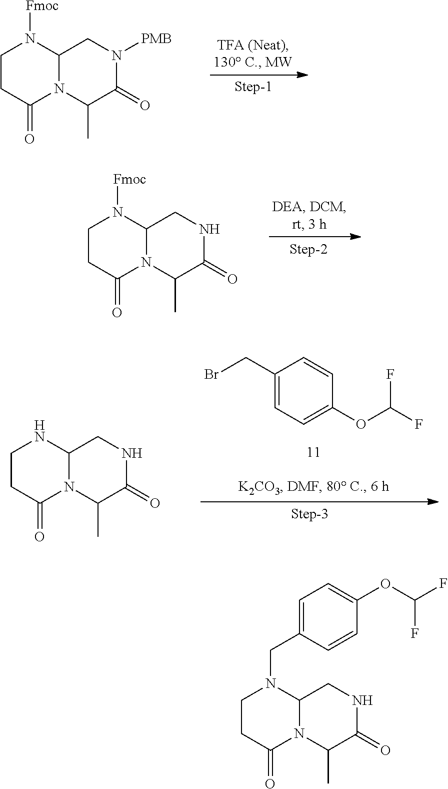

Step 1: Synthesis of (9H-fluoren-9-yl)methyl 6-methyl-4,7-dioxohexahydro-2H-pyrazino[1,2-a]pyrimidine-1(6H)-carboxylate. A solution of (9H-fluoren-9-yl)methyl 8-(4-methoxybenzyl)-6-methyl-4,7-dioxohexahydro-2H-pyrazino[1,2-a]pyri midine-1(6H)-carboxylate (1.0 g, 26.63 mmol) in TFA (10 mL) was stirred at 130° C. for 2 h in microwave. After complete consumption of the starting material (monitored by TLC), the reaction mixture was concentrated under vacuum and the crude product was extracted with ethylacetate (100 ml) and saturated solution of sodium bicarbonate. The organic layer was dried over anhydrous Na₂SO₄ and concentrated under vacuum, and purified by column chromatography (Silica 100-200 mesh; 5% MeOH in DCM) to afford (9H-fluoren-9-yl)methyl 6-methyl-4,7-dioxohexahydro-2H-pyrazino[1,2-a]pyrimidine-1(6H)-carboxylate (300 mg, 42% yield) as a sticky solid. MS (ESI) m/z [M+H]⁺: 406.

Step 2: Synthesis of 6-methylhexahydro-4H-pyrazino[1,2-a]pyrimidine-4,7(6H)-dione. To a solution of (9H-fluoren-9-yl)methyl 6-methyl-4,7-dioxohexahydro-2H-pyrazino[1,2-a]pyrimidine-1(6H)-carboxylate (300 mg, 0.74 mmol) in CH₂C₂ (5 mL) was added diethylamine (6 mL). The reaction mixture was stirred at room temperature for 3 h. After complete consumption of the starting material (monitored by TLC), the reaction mixture was concentrated and the crude product was purified by column chromatography (Silica 100-200 mesh; 10% MeOH in DCM) to afford 6-methylhexahydro-4H-pyrazino[1,2-a]pyrimidine-4,7(6H)-dione (120 mg, 92% yield) as a white solid. MS (ESI) m/z [M+H]⁺: 184.

Step 3: Synthesis of 1-(4-(difluoromethoxy)benzyl)-6-methylhexahydro-4H-pyrazino[1,2-a]pyrimidine-4,7(6H)-dione. To a solution of 6-methylhexahydro-4H-pyrazino[1,2-a]pyrimidine-4,7(6H)-dione (0.700 g, 3.820 mmol) in DMF (8.0 mL) was added K₂CO₃ (1.58 g, 11.46 mmol) at room temperature and stirred for 10 min. To the resulting reaction mixture was added 1-(bromomethyl)-4-(difluoromethoxy)benzene (1.086 g, 4.584 mmol) and the reaction mixture was heated at 80° C. for 6 h. Progress of the reaction was monitored by TLC. After completion, the reaction mixture was cooled to room temperature, quenched with water (50 mL) and extracted with EtOAc (50 mL×2). The combined organic layers were washed with saturated brine solution (20 mL), dried over Na₂SO₄ and concentrated under reduced pressure. The crude product was purified by column chromatography (Silica 100-200 mesh; 5% MeOH in DCM) to afford 1-(4-(difluoromethoxy)benzyl)-6-methylhexahydro-4H-pyrazino[1,2-a]pyrimidine-4,7(6H)-dione (0.550 g, 43.0% yield) as an off-white solid. MS (ESI) m/z [M+H]⁺: 340.34.

### Example S45. General Procedure D for Synthesis of Final Compounds

To a solution of 1-(4-(difluoromethoxy)benzyl)-6-methylhexahydro-4H-pyrazino[1,2-a]pyrimidine-4,7(6H)-dione (0.100 g, 0.2949 mmol) in DMF (2 mL) was added NaH (0.021 g, 0.8847 mmol) at 0° C. followed by the appropriate alkyl halide (2 eq.) and the reaction mixture was allowed to warm to room temperature and stirred for 5 h. Progress of the reaction was monitored by TLC. After completion, the reaction mixture was slowly quenched with saturated solution of aq. NaHCO₃ (2 mL) and extracted with EtOAc (10 mL×2). The combined organic layers were washed with H₂O (5 mL) followed by saturated brine solution (5 mL), dried over Na₂SO₄ and concentrated under reduced pressure. The crude material was purified by combiflash column chromatography (5% MeOH in DCM) to afford the final product.

### Example S46. Synthesis of Compound 37

Compound 37 was synthesized by General Procedure D using 4-bromo-1,1,1-trifluorobutane as the alkyl halide. MS (ESI) m/z [M+H]⁺: 354.2. ¹H NMR (400 MHz, CDCl₃): δ 1.41 (d, J=7.13 Hz, 3H), 1.71-1.86 (m, 2H), 2.01-2.15 (m, 2H), 2.26-2.35 (m, 1H), 2.60-2.67 (m, 1H), 2.89-3.01 (m, 1H), 3.07-3.15 (m, 1H), 3.21-3.34 (m, 2H), 3.46-3.65 (m, 2H), 3.81-3.95 (m, 2H), 4.35-4.41 (m, 1H), 5.20-5.29 (m, 1H), 6.53 (t, J=72.0 Hz, 1H), 7.08-7.16 (m, 2H), 7.30-7.36 (m, 2H).

### Example S47. Synthesis of Compound 38

Compound 38 was synthesized by General Procedure D using (2-bromoethyl)cyclopentane as the alkyl halide. MS (ESI) m/z [M+H]⁺: 436.2. ¹H NMR (400 MHz, CDCl₃) δ 1.01-1.15 (m, 2H), 1.41 (d, J=7.13 Hz, 3H), 1.45-1.62 (m, 8H), 1.66-1.80 (m, 2H), 2.23-2.34 (m, 1H), 2.58-2.72 (m, 1H), 2.89-2.98 (m, 1H), 3.04-3.18 (m, 2H), 3.23-3.33 (m, 1H), 3.43-3.54 (m, 1H), 3.55-3.65 (m, 1H), 3.78-3.93 (m, 1H), 4.31-4.39 (m, 1H), 5.15-5.26 (m, 1H), 6.53 (t, J=72.0 Hz, 1H), 7.13 (d, J=8.50 Hz, 2H), 7.34 (d, J=8.50 Hz, 2H).

### Example S48. Synthesis of Compound 39

Compound 39 was synthesized by General Procedure D using 4-bromobut-1-ene as the alkyl halide. MS (ESI) m/z [M+H]⁺: 394.2. ¹H NMR (400 MHz, CDCl₃) δ 1.41 (d, J=7.13 Hz, 3H), 2.23-2.35 (m, 3H), 2.60-2.71 (m, 1H), 2.92-3.01 (m, 1H), 3.06-3.14 (m, 1H), 3.22-3.46 (m, 3H), 3.53-3.64 (m, 1H), 3.79-3.93 (m, 2H), 4.28-4.38 (m, 1H), 4.91-5.00 (m, 2H), 5.16-5.26 (m, 1H), 5.64-5.76 (m, 1H), 6.52 (t, J=72.0 Hz, 1H), 7.10-7.16 (m, 2H), 7.30-7.36 (m, 2H).

### Example S49. Synthesis of Compound 40

Compound 40 was synthesized by General Procedure D using (2-bromoethyl)cyclobutene as the alkyl halide. MS (ESI) m/z [M+H]⁺: 422.25 ¹H NMR (400 MHz, CDCl₃) δ 1.41 (d, J=7.13 Hz, 3H), 1.56-1.65 (m, 4H), 1.73-1.92 (m, 2H), 1.95-2.07 (m, 2H), 2.14-2.25 (m, 1H), 2.26-2.35 (m, 1H), 2.59-2.72 (m, 1H), 2.91-2.99 (m, 1H), 3.04-3.14 (m, 2H), 3.23-3.43 (m, 2H), 3.53-3.63 (m, 1H), 3.87 (q, J=13.38 Hz, 2H), 4.29-4.39 (m, 1H), 5.17-5.24 (m, 1H), 6.53 (t, J=72.0 Hz, 1H), 7.13 (d, J=8.63 Hz, 2H), 7.30-7.37 (m, 2H).

### Example S50. Synthesis of Compound 41

Compound 41 was synthesized by General Procedure D using 1-bromobutane as the alkyl halide. MS (ESI) m/z [M+H]⁺: 396.05. ¹H NMR (400 MHz, DMSO-d₆) δ 0.86 (t, J=7.34 Hz, 3H), 1.14-1.24 (m, 2H), 1.24-1.30 (m, 2H), 1.38-1.50 (m, 2H), 1.98-2.10 (m, 1H), 2.53-2.61 (m, 2H), 2.64-2.77 (m, 2H), 3.07-3.25 (m, 3H), 3.32-3.41 (m, 1H), 3.62-3.73 (m, 1H), 3.87-3.93 (m, 2H), 4.49-4.58 (m, 1H), 4.84-4.94 (m, 1H), 7.15 (d, J=8.56 Hz, 2H), 7.22 (t, J=72.0 Hz, 1H), 7.43 (d, J=8.56 Hz, 1H).

### Example S51. Synthesis of Compound 52

Compound 52 was synthesized by General Procedure D using 2-trifluromethyl-1-bromoethane as the alkyl halide. MS (ESI) m/z [M+H]⁺: 420.16. ¹H NMR (400 MHz, CDCl₃) δ ppm 7.31-7.38 (m, 2H), 7.11-7.16 (m, 2H), 6.31-6.73 (m, 1H), 5.26 (q, J=7.21 Hz, 1H), 4.23-4.44 (m, 2H), 3.98-4.13 (m, 1H), 3.80-3.93 (m, 3H), 3.59 (t, J=11.07 Hz, 1H), 3.10 (dd, J=11.51, 3.75 Hz, 1H), 2.90-2.99 (m, 1H), 2.62-2.72 (m, 1H), 2.32 (dd, J=4.38, 2.38 Hz, 1H), 2.28 (dd, J=4.31, 2.31 Hz, 1H), 1.48 (d, J=7.25 Hz, 1H), 1.41 (d, J=7.13 Hz, 3H).

### Example S52. General Procedure E for the Synthesis of Final Compounds

To a solution of 6-methyl-8-(2-methylbutyl)hexahydro-4H-pyrazino[1,2-a]pyrimidine-4,7(6H)-dione (0.300 g, 1.184 mmol) in DMF (6 mL) stirred in a flask immersed in an ice/water bath was added cesium carbonate (0.771 g, 2.368 mmol, 2 eq,) followed by the appropriate alkyl halide (1.1 eq.). The flask was removed from the bath and stirred until TLC indicated complete consumption of starting material. The reaction mixture was poured in ice-cold water (70 mL) and aqueous layer was extracted with EtOAc (100 mL). The organic layer was washed with ice cold brine (50 mL×3), dried over anhydrous Na₂SO₄ and concentrated under reduced pressure. The crude product was purified by preparative HPLC to afford to give the final compound.

### Example S53. Synthesis of Compound 42

Compound 42 was synthesized by General Procedure E using 4-(bromomethyl)-2-chloro-1-(trifluoromethyl)benzene as the alkyl halide. MS (ESI) m/z [M+H]⁺: 362.2. ¹H NMR (400 MHz, DMSO-d₆) δ 0.75-0.89 (m, 3H), 0.82-0.87 (m, 3H), 0.96-1.13 (m, 1H), 1.23-1.31 (m, 4H), 1.64-1.75 (m, 1H), 2.06-2.09 (m, 1H), 2.55-2.62 (m, 1H), 2.65-2.76 (m, 1H), 3.05-3.15 (m, 1H), 3.15-3.26 (m, 3H), 3.64-3.74 (m, 1H), 3.84-3.95 (m, 2H), 4.52-4.60 (m, 1H), 4.86-4.94 (m, 1H), 7.17 (t, J=8.76 Hz, 2H), 7.41 (dd, J=8.19, 5.82 Hz, 2H).

### Example S54. Synthesis of Compound 43

Compound 43 was synthesized by General Procedure E using 4-(bromomethyl)-2-chloro-1-(trifluoromethyl)benzene as the alkyl halide. MS (ESI) m/z [M+H]⁺: 446.2. ¹H NMR (400 MHz, DMSO-d₆) 0.72-0.80 (m, 3H), 0.80-0.87 (m, 3H), 0.96-1.10 (m, 1H), 1.21-1.27 (m, 1H), 1.28-1.34 (m, 3H), 1.62-1.79 (m, 1H), 2.00-2.13 (m, 1H), 2.53-2.65 (m, 1H), 2.66-2.76 (m, 1H), 3.00-3.10 (m, 1H), 3.17-3.29 (m, 3H), 3.62-3.72 (m, 1H), 4.00-4.08 (m, 2H), 4.55-4.65 (m, 1H), 4.85-4.95 (m, 1H), 7.52-7.60 (m, 1H), 7.73 (s, 1H), 7.80-7.88 (m, 1H).

### Example S55. Synthesis of Compound 44

To a solution of 6-methyl-8-(2-methylbutyl)hexahydro-4H-pyrazino[1,2-a]pyrimidine-4,7(6H)-dione (0.420 g, 1.657 mmol) and 1H-indole-3-carbaldehyde (0.264 g, 1.823 mmol) in DCE (15 mL) was added acetic acid (1 mL, 1.657 mmol) and heated the reaction mixture at 80° C. for 1 h. To the resulting reaction mixture was added portion wise NaBH₄ (0.188 g, 4.973 mmol) and the reaction mixture was heated at 80° C. and stirred for 4 h. When TLC analysis (5% MeOH in DCM) indicated complete consumption of the starting material the reaction mixture was diluted with water (40 mL) and aqueous layer was extracted with DCM (100 mL). The organic layer was washed with brine (50 mL), dried over anhydrous Na₂SO₄ and concentrated under reduced pressure. The crude obtained was purified by column chromatography (Silica 100-200 mesh; 5% MeOH in DCM) followed by washing with water (30 mL) and dried under reduced pressure to afford compound 44 (0.250 g, 39% yield) as an off white solid. MS (ESI) m/z [M+H]⁺: 383.4. ¹H NMR (400 MHz, DMSO-d₆) δ 0.70 (t, J=7.09 Hz, 3H), 0.75-0.82 (m, 3H), 0.91-1.11 (m, 1H), 1.22-1.31 (m, 3H), 1.57-1.72 (m, 1H), 1.97-2.07 (m, 1H), 2.55-2.70 (m, 2H), 2.83 (dt, J=10.91, 2.74 Hz, 1H), 2.95-3.07 (m, 1H), 3.10-3.26 (m, 3H), 3.54-3.69 (m, 1H), 3.96-4.04 (m, 1H), 4.06-4.15 (m, 1H), 4.54-4.64 (m, 1H), 4.84-4.95 (m, 1H), 6.94-7.02 (m, 1H), 7.04-7.13 (m, 1H), 7.29-7.40 (m, 2H), 7.65 (d, J=7.95 Hz, 1H), 10.95 (s, 1H).

### Example S56. Synthesis of Intermediate Compound 1-(4-fluorobenzyl)-6-methylhexahydro-4H-pyrazino[1,2-a]pyrimidine-4,7(6H) dione

To a solution of 6-methylhexahydro-4H-pyrazino[1,2-a]pyrimidine-4,7(6H)-dione (250 mg, 1.40 mmol) in DMF (3 mL) was added potassium carbonate (580 mg, 4.20 mmol) followed by 4-fluorobenzylbromide (0.320 g, 1.70 mmol) and stirred at 80° C. temperature for 3 h. After completion, the reaction mixture was monitored by TLC (5% MeOH in DCM). The reaction mixture was poured in ice-cold water (50 mL) and aqueous layer was extracted with EtOAc (50 mL). The organic layer was dried over anhydrous Na₂SO₄ and concentrated under reduced pressure. The crude obtained was purified by column chromatography (Silica 100-200 mesh; 5% MeOH in DCM) to afford 1-(4-fluorobenzyl)-6-methylhexahydro-4H-pyrazino[1,2-a]pyrimidine-4,7(6H) dione (160 mg, 70% yield) as a white solid. MS (ESI) m/z [M+H]⁺: 292.

### Example S57. Synthesis of Compound 45

To a solution of 1-(4-fluorobenzyl)-6-methylhexahydro-4H-pyrazino[1,2-a]pyrimidine-4,7(6H) dione (80 mg, 0.2739 mmol) in DMF (3 mL) under an ice cold bath at 0° C. was added NaH (20 mg, 0.2739 mmol) and stirred for 20 min then added (2-bromoethyl)cyclobutane (67 mg, 0.41 mmol) after 3 h. After complete consumption of starting material (monitored by TLC), the reaction mixture was quenched with ice cold water and extracted with ethyl acetate. The organic layer was dried over anhydrous Na₂SO₄ and concentrated under reduced pressure. The crude obtained was purified by column chromatography (Silica 100-200 mesh; 5% MeOH in DCM) to afford 8-(2-cyclobutylethyl)-1-(4-fluorobenzyl)-6-methylhexahydro-4H-pyrazino[1,2-a]pyrimidine-4,7(6H)-dione (13 mg, 16% yield) as a gummy liquid. MS (ESI) m/z [M+H]⁺: 374. ¹H NMR (400 MHz, CD3Cl₃): δ 7.30-7.40 (m, 2H), 7.00-7.10 (m, 2H), 5.15-5.25 (m, 1H), 4.25-4.35 (m, 1H), 3.80-3.95 (m, 2H), 3.55-3.65 (m, 1H), 3.25-3.45 (m, 2H), 3.05-3.20 (m, 2H), 2.90-3.0 (m, 1H), 2.60-2.70 (m, 1H), 2.15-2.40 (m, 2H), 1.75-2.10 (m, 4H), 1.55-1.65 (m, 4H), 1.20-1.30 (m, 3H).

### Example S58. Synthesis of Compound 46

To a solution of 1-(4-fluorobenzyl)-6-methylhexahydro-4H-pyrazino[1,2-a]pyrimidine-4,7(6H) dione (80 mg, 0.2739 mmol) in DMF (3 mL) under an ice cold bath at 0° C. was added NaH (20 mg, 0.2739 mmol) and stirred for 20 min, then was added (2-bromoethyl)cyclopentane (72 mg, 0.41 mmol) after 3 hours, completion of starting material monitored by TLC, the reaction mixture was quenched with ice cold water and extracted with ethyl acetate. The organic layer was dried over anhydrous Na₂SO₄ and concentrated under reduced pressure. The crude obtained was purified by column chromatography (Silica 100-200 mesh; 5% MeOH in DCM) to afford 8-(2-cyclopentylethyl)-1-(4-fluorobenzyl)-6-methylhexahydro-4H-pyrazino[1,2-a]pyrimidine-4,7(6H)-dione as a gummy liquid.

### Example S59. Synthesis of Intermediate Compound 6-(fluoromethyl)-8-(2-methylbutyl)hexahydro-4H-pyrazino[1,2-a]pyrimidine-4,7(6H)-dione Hydrochloride salt

Step 1: Synthesis of N-(2,2-diethoxyethyl)-2-methylbutan-1-amine. To stirred neat 2,2-diethoxyethan-1-amine (20.0 g, 0.137 mmol) was added 2-methylbutanal (11.60 g, 0.137 mmol) at room temperature and the reaction mixture was heated to 100° C. for 3 h. To the resulting reaction mixture was slowly added ethanol (200 mL) followed by NaBH₄ (15.40 g, 0.413 mmol) at room temperature and the reaction mixture was stirred for 16 h. After complete consumption of starting material (monitored by TLC). The reaction mixture was cooled to room temperature and slowly quenched with a saturated solution of NH₄Cl (100 mL). The aq. layer was extracted with EtOAc (200 mL×2). The combined organic layer was washed with brine (400 mL), dried over Na₂SO₄, filtered and concentrated under reduced pressure to get crude compound. The crude obtained was purified by column chromatography (silica 100-200 mesh; 10% MeOH in DCM) to obtain N-(2,2-diethoxyethyl)-2-methylbutan-1-amine (25.8 g, 88% yield) colorless liquid. MS (ESI) m/z [M+H]: 204.3. ¹H NMR (400 MHz, DMSO-d₆) δ 0.80-0.89 (m, 6H) 1.11 (t, J=6.98 Hz, 6H) 1.35-1.48 (m, 2H) 2.28-2.32 (m, 1H) 2.41-2.45 (m, 1H) 2.55 (d, J=5.49 Hz, 2H) 3.42-3.52 (m, 2H) 3.57-3.65 (m, 2H) 4.49 (t, J=5.49 Hz, 1H).

Step 2: (9H-fluoren-9-yl)methyl (1-((2,2-diethoxyethyl)(2-methylbutyl)amino)-3-hydroxy-1-oxopropan-2-yl)carbamate. To a stirred solution of (((9H-fluoren-9-yl)methoxy)carbonyl)serine (15.0 g, 45.81 mmol) in dry DMF (150 mL) maintained at 0° C. was added HATU (26.0 g, 68.80 mmol), DIPEA (23.92 mL, 137.61 mmol) followed by N-(2,2-diethoxyethyl)-2-methylbutan-1-amine (12.10 g, 59.63 mmol). The reaction mixture was stirred at room temperature for 4 h. After complete consumption of starting material, the reaction mixture was quenched with ice cold water (500 mL) and the aqueous layer was extracted with EtOAc (250 mL×2). The combined organic layer was washed with cold H₂O (200 mL) followed by brine (200 mL), dried over Na₂SO₄ and concentrated under reduced pressure to provide the crude product. The crude material was purified by column chromatography (Silica 100-200 mesh; 80% EtOAc in hexanes) to afford (9H-fluoren-9-yl)methyl (1-((2,2-diethoxyethyl)(2-methylbutyl)amino)-3-hydroxy-1-oxopropan-2-yl)carbamate (21.0 g, 89.43% yield) as yellow sticky solid. MS (ESI) m/z [M+Na]⁺: 535.35.

Step 3: Synthesis of 2-amino-N-(2,2-diethoxyethyl)-3-hydroxy-N-(2-methylbutyl)propenamide. To a stirred solution of (9H-fluoren-9-yl)methyl (1-((2,2-diethoxyethyl)(2-methylbutyl)amino)-3-hydroxy-1-oxopropan-2-yl)carbamate (21.0 g, 41.01 mmol) in dry DCM (110 mL) maintained at 0° C. was added diethylamine (58 mL, 2.80 volume) and reaction mixture was stirred at room temperature for 3 h. After complete consumption of starting material (monitored by TLC), the reaction mixture was concentrated under reduced pressure to get crude product. The crude obtained was purified by column chromatography (Silica 100-200 mesh; 5% MeOH in DCM) to afford 2-amino-N-(2,2-diethoxyethyl)-3-hydroxy-N-(2-methylbutyl)propenamide (9.50 g, 80% yield) as yellow sticky solid. MS (ESI) m/z [M+H]⁺: 291.4.

Step 4: Synthesis of (9H-fluoren-9-yl)methyl (3-((1-((2,2-diethoxyethyl)(2-methylbutyl)amino)-3-hydroxy-1-oxopropan-2-yl)amino)-3-oxopropyl)carbamate. To a stirred solution of 3-((((9H-fluoren-9-yl)methoxy)carbonyl)amino)propanoic acid (9.50 g, 30.54 mmol) in dry DMF (95 mL) maintained at 0° C. was added HATU (17.40 g, 45.81 mmol), DIPEA (16.0 mL, 91.62 mmol) followed by 2-amino-N-(2,2-diethoxyethyl)-3-hydroxy-N-(2-methylbutyl)propanamide (13.20 g, 45.81 mmol) at room temperature and the reaction mixture was stirred for 16 h. After completion, the reaction mixture was quenched with ice cold water (200 mL) and the aqueous layer was extracted with EtOAc (200 mL×2). The organic layer was washed with cold H₂O (500 mL) followed by saturated brine (200 mL), dried over Na₂SO₄ and concentrated under reduced pressure. The crude compound was purified by column chromatography (Silica 100-200 mesh; 80% EtOAc in Hexanes) to afford (9H-fluoren-9-yl)methyl (3-((1-((2,2-diethoxyethyl)(2-methylbutyl)amino)-3-hydroxy-1-oxopropan-2-yl)amino)-3-oxopropyl)carbamate (8.0 g, 31.0% yield) as a viscous yellow oil. MS (ESI) m/z [M−H]: 582.2.

Step 5: Synthesis of (9H-fluoren-9-yl)methyl 6-(hydroxymethyl)-8-(2-methylbutyl)-4,7-dioxohexahydro-2H-pyrazino[1,2-a]pyrimidine-1(6H)-carboxylate. A stirred solution of (9H-fluoren-9-yl)methyl (3-((1-((2,2-diethoxyethyl)(2-methylbutyl)amino)-3-hydroxy-1-oxopropan-2-yl)amino)-3-oxopropyl)carbamate (8.0 g, 13.77 mmol) in formic acid (48.0 mL, 6.0 volume) at room temperature and reaction mixture was stirred for 16 h. After completion, the reaction mixture was concentrated under reduced pressure to afford (9H-fluoren-9-yl)methyl 6-(hydroxymethyl)-8-(2-methylbutyl)-4,7-dioxohexahydro-2H-pyrazino[1,2-a]pyrimidine-1(6H)-carboxylate (6.0 g, crude) as brown semi-solid. The crude compound was used as such for next reaction without further purification. MS (ESI) m/z [M+H]⁺: 492.2.

Step 6: Synthesis of 6-(hydroxymethyl)-8-(2-methylbutyl)hexahydro-4H-pyrazino[1,2-a]pyrimidine-4,7(6H)-dione. To a solution of (9H-fluoren-9-yl)methyl 6-(hydroxymethyl)-8-(2-methylbutyl)-4,7-dioxohexahydro-2H-pyrazino[1,2-a]pyrimidine-1(6H)-carboxylate (6.0 g, 12.20 mmol) in CH₂Cl₂ (36.0 mL) was added diethylamine (18.0 mL) at 0° C. and the reaction mixture was stirred at room temperature for 3 h. After complete consumption of the starting material (monitored by TLC), the reaction mixture was concentrated under reduced pressure to obtain the crude compound. The crude material was purified by column chromatography (Silica 100-200 mesh; 5% MeOH in DCM) to afford 6-(hydroxymethyl)-8-(2-methylbutyl)hexahydro-4H-pyrazino[1,2-a]pyrimidine-4,7(6H)-dione (3.0 g, 93.75% yield) as a viscous colorless oil. MS (ESI) m/z [M+H]⁺: 270.20.

Step 7: Synthesis of tert-butyl 6-(hydroxymethyl)-8-(2-methylbutyl)-4,7-dioxohexahydro-2H-pyrazino[1,2-a]pyrimidine-1(6H)-carboxylate. To a solution of 6-(hydroxymethyl)-8-(2-methylbutyl)hexahydro-4H-pyrazino[1,2-a]pyrimidine-4,7(6H)-dione (3.0 g, 11.15 mmol) in CH₂Cl₂ (60 mL) was added triethylamine (4.5 mL, 33.45 mmol) followed by Boc anhydride (3.78 mL, 16.72 mmol) at 0° C. and the reaction mixture was stirred at room temperature for 16 h. After complete consumption of the starting material (monitored by TLC), the reaction mixture was slowly quenched with ice cold water (30 mL) and extracted with DCM (40 mL). The organic layer was washed with brine (30 mL), dried over Na₂SO₄ and concentrated under reduced pressure. The crude compound was purified by column chromatography (Silica 100-200 mesh; 10% MeOH in DCM) to afford tert-butyl 6-(hydroxymethyl)-8-(2-methylbutyl)-4,7-dioxohexahydro-2H-pyrazino[1,2-a]pyrimidine-1(6H)-carboxylate (8.0 g, 31.0% yield) as a viscous yellow oil. MS (ESI) m/z [M+H]⁺: 370.25.

Step 8: Synthesis of tert-butyl 6-(fluoromethyl)-8-(2-methylbutyl)-4,7-dioxohexahydro-2H-pyrazino[1,2-a]pyrimidine-1(6H)-carboxylate. To a solution of 6-(hydroxymethyl)-8-(2-methylbutyl)-4,7-dioxohexahydro-2H-pyrazino[1,2-a]pyrimidine-1(6H)-carboxylate (1.50 g, 4.065 mmol) in DCM (30 mL) was added DAST (1.97 g, 12.19 mmol) at −78° C. and stirred for 15 min. The reaction mixture was allowed to warm to room temperature and stirred for 3 h. After completion of the reaction (monitored by TLC), the reaction mixture was quenched with saturated NaHCO₃ solution (15 mL) and the aqueous layer was extracted with EtOAc (100 mL×2). The combined organic layer was washed with saturated brine (50 mL), dried over Na₂SO₄ and concentrated under reduced pressure to obtain crude compound. The crude material was purified by column chromatography (Silica 100-200 mesh; 5% MeOH in DCM) to afford tert-butyl 6-(fluoromethyl)-8-(2-methylbutyl)-4,7-dioxohexahydro-2H-pyrazino[1,2-a]pyrimidine-1(6H)-carboxylate (0.800 g, 72.0% yield) as a colorless viscous oil. MS (ESI) m/z [M+H]⁺: 372.2.

Step 9: Synthesis of 6-(fluoromethyl)-8-(2-methylbutyl)hexahydro-4H-pyrazino[1,2-a]pyrimidine-4,7(6H)-dione Hydrochloride salt. To a stirred solution of tert-butyl 6-(fluoromethyl)-8-(2-methylbutyl)-4,7-dioxohexahydro-2H-pyrazino[1,2-a]pyrimidine-1(6H)-carboxylate (1.0 g, 2.695 mmol) in 1,4-dioxane (5 mL) was added 4 M HCl in dioxane (5 mL) at 0° C. and the reaction mixture was stirred at room temperature for 3 h. After complete consumption of the starting material (monitored by TLC), the reaction mixture was quenched with saturated solution of sodium bicarbonate (10 mL) and extracted with ethyl acetate (20 mL×3). The combined organic layer was washed with saturated brine (10 mL), dried over Na₂SO₄ and concentrated under reduced pressure to afford 6-(fluoromethyl)-8-(2-methylbutyl)hexahydro-4H-pyrazino[1,2-a]pyrimidine-4,7(6H)-dione hydrochloride salt (0.630 g, crude) as a brown sticky oil. MS (ESI) m/z [M+H]⁺ free base: 271.00.

### Example S60. Synthesis of Compound 47

To a solution of 6-(fluoromethyl)-8-(2-methylbutyl)hexahydro-4H-pyrazino[1,2-a]pyrimidine-4,7(6H)-dione hydrochloride salt (0.150 g, 0.550 mmol) in DMF (1.5 mL) was added K₂CO₃ (0.381 g, 2.760 mmol) followed by 1-(bromomethyl)-4-(difluoromethoxy)benzene (0.261 g, 1.100 mmol) and the reaction mixture was stirred at room temperature for 16 h. After completion (monitored by TLC), the reaction mixture was slowly quenched with ice cold water (6 mL) and extracted with EtOAc (20 mL×3). The combined organic layer was washed with saturated brine solution (10 mL), dried over Na₂SO₄ and concentrated under reduced pressure to get crude compound. The crude compound was purified by Prep HPLC to afford 1-(4-(difluoromethoxy)benzyl)-6-(fluoromethyl)-8-(2-methylbutyl)hexahydro-4H-pyrazino[1,2-a]pyrimidine-4,7(6H)-dione (0.040 g, 17.0% yield) as a white solid. MS (ESI) m/z [M+H]⁺: 428.10. ¹H NMR (400 MHz, CDCl₃) δ 7.34 (d, J=8.01, 2 H), 7.11 (d, J=8.01, 2H), 6.32-6.69 (m, 1H), 5.14-5.25 (m, 2H), 4.60-4.76 (m, 2H), 3.84-3.97 (m, 2H), 3.35-3.45 (m, 2H), 3.12-3.40 (m, 4H), 2.85-3.05 (m, 1H), 2.65-2.75 (m, 1H), 2.29-2.34 (m, 1H), 1.65-1.75 (m, 1H), 1.30-1.40 (m, 1H), 1.05-1.18 (m, 1H), 0.80-0.90 (m, 6H).

### Example S61. Synthesis of Compound 48

To a solution of 6-(fluoromethyl)-8-(2-methylbutyl)hexahydro-4H-pyrazino[1,2-a]pyrimidine-4,7(6H)-dione hydrochloride salt (0.340 g, 1.253 mmol) in DMF (3.4 mL) was added Cs₂CO₃ (0.814 g, 2.506 mmol) followed by 1-(bromomethyl)-4-(trifluoromethyl)benzene (0.598 g, 2.506 mmol), and reaction mixture was stirred at room temperature for 16 h. After completion (monitored by TLC), the reaction mixture was slowly quenched with ice cold water (6 mL) and extracted with EtOAc (20 mL×3). The combined organic layer was washed with saturated brine solution (10 mL), dried over Na₂SO₄ and concentrated under reduced pressure to get crude compound. The crude compound was purified by prep HPLC to afford 6-(fluoromethyl)-8-(2-methylbutyl)-1-(4-(trifluoromethyl)benzyl)hexahydro-4H-pyrazino[1,2-a]pyrimidine-4,7(6H)-dione (0.045 g, 8.0% yield) as a white solid. MS (ESI) m/z [M+H]⁺: 430.10. ¹H NMR (400 MHz, CDCl₃) δ 7.34 (d, J=8.01, 2H), 7.11 (d, J=8.01, 2H), 5.14-5.25 (m, 2H), 4.60-4.76 (m, 2H), 3.84-3.97 (m, 2H), 3.35-3.45 (m, 2H), 3.12-3.40 (m, 4H), 2.85-3.05 (m, 1H), 2.65-2.75 (m, 1H), 2.29-2.34 (m, 1H), 1.65-1.75 (m, 1H), 1.30-1.40 (m, 1H), 1.05-1.18 (m, 1H), 0.80-0.90 (m, 6H).

### Example S62. Synthesis of Intermediate Compound methyl 2-(1-(4-(difluoromethoxy)benzyl)-8-(2-methylbutyl)-4,7-dioxooctahydro-2H-pyrazino[1,2-a]pyrimidin-6-yl)acetate

Step 1: Synthesis of methyl 3-((((9H-fluoren-9-yl)methoxy)carbonyl)amino)-4-((2,2-diethoxyethyl)(2-methylbutyl)amino)-4-oxobutanoate. To a solution 2-((((9H-fluoren-9-yl)methoxy)carbonyl)amino)-4-methoxy-4-oxobutanoic acid (1.90 g, 9.475 mmol) stirred at 0° C. in dry DMF (30 mL) was added HATU (3.60 g, 1.137 mmol) followed by DIPEA (2.70 mL, 1.895 mmol), and the reaction mixture was stirred at same temperature for 10 min. To the resulting reaction mixture was added N-(2,2-diethoxyethyl)-2-methylbutan-1-amine (3.50 g, 9.475 mmol), then the mixture was allowed to warm to room temperature and stirred for 6 h. After complete consumption of the starting material (monitored by TLC), the reaction mixture was quenched with ice cold water (100 mL) and the aqueous layer was extracted with EtOAc (50 mL×2). The combined organic layer was washed with cold H₂O (50 mL) followed by brine (50 mL), dried over Na₂SO₄ and concentrated under reduced pressure to get crude product. The crude material was purified by CombiFlash column chromatography using 50% EtOAc in n-hexanes to afford methyl 3-((((9H-fluoren-9-yl)methoxy)carbonyl)amino)-4-((2,2-diethoxyethyl)(2-methylbutyl)amino)-4-oxobutanoate (4.30 g, 83.0% yield) as a white solid. MS (ESI) m/z [M+H-EtOH]⁺: 509.2.

Step 2: Synthesis of methyl 3-amino-4-((2,2-diethoxyethyl)(2-methylbutyl)amino)-4-oxobutanoate. To a solution of methyl 3-((((9H-fluoren-9-yl)methoxy)carbonyl)amino)-4-((2,2-diethoxyethyl)(2-methylbutyl)amino)-4-oxobutanoate (1.36 g, 2.451 mmol) in CH₂Cl₂ (27.0 mL) was added diethylamine (1.53 mL, 14.71 mmol) at room temperature and the reaction mixture was stirred for 3 h. After complete consumption of the starting material (monitored by TLC), the reaction mixture was concentrated under reduced pressure to obtained crude compound. The crude compound was purified by CombiFlash column chromatography using 5% MeOH in DCM to afford methyl 3-amino-4-((2,2-diethoxyethyl)(2-methylbutyl)amino)-4-oxobutanoate (0.700 g, 86% yield) as yellow viscous liquid. MS (ESI) m/z [M+H-EtOH]⁺: 287.68.

Step 3: Synthesis of methyl 3-(3-((((9H-fluoren-9-yl)methoxy)carbonyl)amino)propanamido)-4-((2,2-diethoxyethyl)(2-methylbutyl)amino)-4-oxobutanoate. To a stirred solution of 3-((((9H-fluoren-9-yl)methoxy)carbonyl)amino)propanoic acid (0.490 g, 1.594 mmol) in dry DMF (10 mL) maintained at 0° C. was added HATU (0.720 g, 1.913 mmol), DIPEA (0.555 mL, 3.188 mmol) followed by the addition of methyl 3-amino-4-((2,2-diethoxyethyl)(2-methylbutyl)amino)-4-oxobutanoate (0.530 g, 1.594 mmol). The reaction mixture was allowed to warm to room temperature and stirred for 6 h. After completion, the reaction mixture was quenched with ice cold water (20 mL) and the aqueous layer was extracted with EtOAc (20 mL×2). The organic layer was washed with cold H₂O (10 mL) followed by saturated brine (20 mL), dried over Na₂SO₄ and concentrated under reduced pressure. The crude compound was purified by Combiflash column chromatography using 5% MeOH in DCM to afford methyl 3-(3-((((9H-fluoren-9-yl)methoxy)carbonyl)amino)propanamido)-4-((2,2-diethoxyethyl)(2-methylbutyl)amino)-4-oxobutanoate (0.630 g, 70% yield) as an off-white solid. MS (ESI) m/z [M+H-EtOH]⁺: 580.20.

Step 4: Synthesis of (9H-fluoren-9-yl)methyl 6-(2-methoxy-2-oxoethyl)-8-(2-methylbutyl)-4,7-dioxohexahydro-2H-pyrazino[1,2-a]pyrimidine-1(6H)-carboxylate. To a stirred solution of methyl 3-(3-((((9H-fluoren-9-yl)methoxy)carbonyl)amino)propanamido)-4-((2,2-diethoxyethyl)(2-methylbutyl)amino)-4-oxobutanoate (0.300 g, 0.4794 mmol) was added formic acid (1.5 mL) at room temperature and the reaction mixture was stirred for 16 h. After completion, the reaction mixture was concentrated and the crude obtained was purified by column chromatography (Silica 100-200 mesh; 0-5% MeOH in DCM) to afford (9H-fluoren-9-yl)methyl 6-(2-methoxy-2-oxoethyl)-8-(2-methylbutyl)-4,7-dioxohexahydro-2H-pyrazino[1,2-a]pyrimidine-1(6H)-carboxylate (0.200 g, 80% yield) as a yellow solid. MS (ESI) m/z [M+H]⁺: 534.67

Step 5: Synthesis of methyl 2-(8-(2-methylbutyl)-4,7-dioxooctahydro-2H-pyrazino[1,2-a]pyrimidin-6-yl)acetate. To a solution of (9H-fluoren-9-yl)methyl 6-(2-methoxy-2-oxoethyl)-8-(2-methylbutyl)-4,7-dioxohexahydro-2H-pyrazino[1,2-a]pyrimidine-1(6H)-carboxylate (0.240 g, 0.4499 mmol) in CH₂C₁₂ (0.5 mL) was added diethylamine (0.280 mL) and the reaction mixture was stirred at room temperature for 3 h. After complete consumption of the starting material (monitored by TLC), the reaction mixture was concentrated and the crude material was purified by combiflash column chromatography using 0-5% MeOH in DCM to afford methyl 2-(8-(2-methylbutyl)-4,7-dioxooctahydro-2H-pyrazino[1,2-a]pyrimidin-6-yl)acetate (0.130 g, 93% yield) as white solid. MS (ESI) m/z [M−H]⁺: 310.4.

Step 6: Synthesis of methyl methyl 2-(1-(4-(difluoromethoxy)benzyl)-8-(2-methylbutyl)-4,7-dioxooctahydro-2H-pyrazino[1,2-a]pyrimidin-6-yl)acetate. To a solution of methyl 2-(8-(2-methylbutyl)-4,7-dioxooctahydro-2H-pyrazino[1,2-a]pyrimidin-6-yl)acetate (3.08 g, 9.890 mmol) in DMF (30 mL) was added K₂CO₃ (4.10 g, 29.66 mmol) at room temperature, and reaction mixture stirred at 80° C. for 15 min. To the resulting reaction mixture was added 1-(bromomethyl)-4-(difluoromethoxy)benzene (3.48 g, 14.36 mmol) and the stirred mixture was heated to 80° C. for 2 h. After completion, the reaction mixture was quenched with ice cold water (200 mL) and the aqueous layer was extracted with EtOAc (200 mL×2). The organic layer was washed with cold H₂O (200 mL) followed by saturated brine (150 mL), dried over Na₂SO₄ and concentrated under reduced pressure. The crude compound obtained was purified by Combiflash column chromatography (5% MeOH in DCM) to afford methyl 2-(1-(4-(difluoromethoxy)benzyl)-8-(2-methylbutyl)-4,7-dioxooctahydro-2H-pyrazino[1,2-a]pyrimidin-6-yl)acetate (2.20 g, 48% yield) as a yellow solid. MS (ESI) m/z [M-CH₃]⁺: 454.10.

### Example S63. Synthesis of Compound 49

To a solution of methyl 2-(1-(4-(difluoromethoxy)benzyl)-8-(2-methylbutyl)-4,7-dioxooctahydro-2H-pyrazino[1,2-a]pyrimidin-6-yl)acetate (2.20 g, 4.705 mmol) in THE (22.0 mL) was added NaOH (0.560 g, 14.11 mmol) followed by water (4 mL) and the reaction mixture was stirred at room temperature for 3 h. Progress of the reaction was monitored by TLC. After completion, the reaction mixture was concentrated under reduced pressure. The crude residue was dissolved in water (10 mL), slowly acidified with 6N HCl (10 mL) and stirred for 5 min. The obtained solid precipitate was filtered through a Buchner funnel and dried under reduced pressure to afford 2-(1-(4-(difluoromethoxy)benzyl)-8-(2-methylbutyl)-4,7-dioxooctahydro-2H-pyrazino[1,2-a]pyrimidin-6-yl)acetic acid (0.85 g, 40% yield) as a white solid. MS (ESI) m/z [M+H]⁺: 454.10. ¹H NMR (400 MHz, CDCl₃) δ 7.28-7.38 (m, 2H), 7.11 (d, J=7.99 Hz, 2H), 6.33-6.71 (m, 1H), 5.36-5.40 (m, 1H), 4.70-4.80 (m, 1H), 4.65-4.75 (m, 1H), 3.80-4.00 (m, 2H), 3.55-3.65 (m, 1H), 3.35-3.45 (m, 1H), 2.85-3.30 (m, 6H), 2.70-2.80 (m, 1H), 2.25-2.35 (m, 1H), 1.65-1.76 (m, 1H), 1.25-1.35 (m, 1H), 1.10-1.20 (m, 1H), 0.8-0.9 (m, 6H).

### Example S64. Synthesis of Compound 50

To a solution of 2-(1-(4-(difluoromethoxy)benzyl)-8-(2-methylbutyl)-4,7-dioxooctahydro-2H-pyrazino[1,2-a]pyrimidin-6-yl)acetic acid (0.470 g, 1.036 mmol) in THE (5 mL) was added 1,1′-carbonyldiimidazole (0.500 g, 3.109 mmol) at room temperature and the reaction mixture was stirred for 15 min. To the resulting reaction mixture was added aq. NH₃ (10 mL) and reaction mixture was stirred at room temperature for 3 h. Progress of the reaction was monitored by TLC. After completion, the reaction mixture was slowly quenched with ice cold water (6 mL) and extracted with EtOAc (20 mL×3). The combined organic layer was washed with saturated brine solution (10 mL), dried over Na₂SO₄ and concentrated under reduced pressure to provide the crude compound. The crude compound obtained was purified by Combiflash column chromatography using 5% MeOH in DCM followed by PREP HPLC to afford 2-(1-(4-(difluoromethoxy)benzyl)-8-(2-methylbutyl)-4,7-dioxooctahydro-2H-pyrazino[1,2-a]pyrimidin-6-yl)acetamide (0.070 g, 15% yield) as a white solid. MS (ESI) m/z [M+H]⁺: 453.20. ¹H NMR (400 MHz, CDCl₃) δ 7.30-7.40 (m, 2H), 7.05-7.15 (m, 2H), 6.39-6.70 (m, 1H), 5.20-5.40 (m, 2H), 4.75-4.85 (m, 1H), 3.95-4.05 (m, 1H), 3.75-3.85 (m, 1H), 3.50-3.60 (m, 1H), 3.30-3.40 (m, 1H), 3.05-3.25 (m, 2H), 2.85-2.95 (m, 2H), 2.55-2.70 (m, 1H), 2.25-2.35 (m, 1H), 1.70-1.80 (m, 2H), 1.30-1.40 (m, 2H), 1.05-1.20 (m, 2H), 0.75-0.90 (m, 6H).

### Example S65. Synthesis of Intermediate Compound 1-(3-chloro-4-(trifluoromethyl)benzyl)-6-methylhexahydro-4H-pyrazino[1,2-a]pyrimidine-4,7(6H)-dione

To a solution of 6-methylhexahydro-4H-pyrazino[1,2-a]pyrimidine-4,7(6H)-dione (500 mg, 2.732 mmol) in DMF (7 mL) was added potassium carbonate (1.13 g, 8.196 mmol) followed by 4-(bromomethyl)-2-chloro-1-(trifluoromethyl)benzene (0.894 g, 3.278 mmol) and stirred at 80° C. temperature for 12 h. After completion of the reaction, monitored by TLC (5% MeOH in DCM). The reaction mixture was poured in ice-cold water (50 mL) and the aqueous layer was extracted with EtOAc (50 mL). The organic layer was dried over anhydrous Na₂SO₄ and concentrated under reduced pressure. The crude obtained was purified by column chromatography (Silica 100-200 mesh; 5% MeOH in DCM) to afford 1-(3-chloro-4-(trifluoromethyl)benzyl)-6-methylhexahydro-4H-pyrazino[1,2-a]pyrimidine-4,7(6H)-dione (320 mg, 42% yield) as a white solid. MS (ESI) m/z [M+H]⁺: 376.34.

### Example S66. General Procedure F for the Synthesis of Final Compounds

To a solution of 1-(3-chloro-4-(trifluoromethyl)benzyl)-6-methylhexahydro-4H-pyrazino[1,2-a]pyrimidine-4,7(6H)-dione (150 mg, 0.400 mmol) in DMF (2 mL) at 0° C. was added Cs₂CO₃ (4 eq) and stirred for 20 min, then was added the appropriate alkyl halide (1.2 eq) at room temperature and the reaction mixture was heated at 80° C. and stirred for 12 h. After consumption of starting material (monitored by TLC), the reaction mixture was quenched with ice cold water and extracted with ethyl acetate. The organic layer was dried over anhydrous Na₂SO₄ and concentrated under reduced pressure. The crude obtained was purified by column chromatography (Silica 100-200 mesh; 5% MeOH in DCM) to give the final compounds.

### Example S67. Synthesis of Compound 51

Compound 51 was synthesized by General Procedure F using (2-bromoethyl)cyclobutane as the alkyl halide. MS (ESI) m/z [M+H]⁺: 458.2. ¹H NMR (400 MHz, CDCl₃) δ ppm 7.70 (d, J=8.07 Hz, 1H), 7.54 (s, 1H), 7.33 (d, J=8.68 Hz, 1H), 4.34 (dd, J=10.64, 3.55 Hz, 1H), 3.87-3.99 (m, 2H), 3.61 (t, J=11.13 Hz, 1H), 3.25-3.42 (m, 2H), 3.08-3.19 (m, 2H), 2.89-2.98 (m, 1H), 2.64-2.74 (m, 1H) 2.29-2.38 (m, 1H) 2.17-2.27 (m, 1H) 1.97-2.09 (m, 2H) 1.72-1.92 (m, 3H) 1.58-1.66 (m, 4H) 1.55 (br. s, 3H).

### Example S68. Synthesis of Compound 54

Compound 54 was synthesized by General Procedure F using (2-bromoethyl)cyclopentane as the alkyl halide. MS (ESI) m/z [M+H]⁺: 472.15. ¹H NMR (400 MHz, CDCl₃) δ ppm 7.69 (d, J=8.11 Hz, 1H) 7.52-7.56 (m, 1H) 7.33 (d, J=7.89 Hz, 1H), 5.23 (q, J=7.23 Hz, 1H), 4.36 (dd, J=10.52, 3.29 Hz, 1H), 3.87-3.98 (m, 2H), 3.63 (t, J=11.07 Hz, 1H), 3.46-3.56 (m, 1H), 3.25-3.35 (m, 1H), 3.12-3.23 (m, 2H), 2.87-2.97 (m, 1H), 2.60-2.70 (m, 1H), 2.29-2.37 (m, 1H), 1.66-1.82 (m, 2H), 1.54-1.63 (m, 1H), 1.51 (d, J=, 2.63 Hz, 2H), 1.42 (d, J=7.23 Hz, 3H), 1.26 (br. s, 2H) 1.04-1.16 (m, 2H) 0.80-0.92 (m, 2H).

### Example S69. Synthesis of Compound 53

Step 1: Synthesis of (9H-fluoren-9-yl)methyl (1-((2,2-diethoxyethyl)(2-methylbutyl)amino)-4-methyl-1-oxopentan-2-yl)carbamate. To a stirred solution of (((9H-fluoren-9-yl)methoxy)carbonyl)leucine (20.0 g, 56.58 mmol) in dry DMF (200 mL) was added HATU (21.50 g, 56.58 mmol) followed by DIPEA (10.62 mL, 61.10 mmol) at 0° C. and the reaction mixture was stirred at same temperature for 10 min. To the resulting reaction mixture was added N-(2,2-diethoxyethyl)-2-methylbutan-1-amine (11.48 g, 56.58 mmol) at room temperature and the reaction mixture was stirred for 3 h. After complete consumption of the starting material (monitored by TLC), the reaction mixture was quenched with ice cold water (100 mL) and the aqueous layer was extracted with EtOAc (50 mL×4). The combined organic layers were washed with cold H₂O (50 mL×2) followed by brine (50 mL), dried over Na₂SO₄ and concentrated under reduced pressure to get crude product. The crude product was purified by CombiFlash column chromatography using 5% MeOH in DCM to afford (9H-fluoren-9-yl)methyl (1-((2,2-diethoxyethyl)(2-methylbutyl)amino)-4-methyl-1-oxopentan-2-yl)carbamate (14.5 g, 47.57% yield) as a white solid. MS (ESI) m/z [M+H]⁺: 539.04.

Step 2: Synthesis of 2-amino-N-(2,2-diethoxyethyl)-4-methyl-N-(2-methylbutyl)pentanamide. To a solution of (9H-fluoren-9-yl)methyl (1-((2,2-diethoxyethyl)(2-methylbutyl)amino)-4-methyl-1-oxopentan-2-yl)carbamate (8.50 g, 15.77 mmol) in CH₂C₁₂ (50 mL) was added diethylamine (16 mL, 157.7 mmol) at room temperature and the reaction mixture was stirred for 3 h. After complete consumption of the starting material (monitored by TLC), the reaction mixture was concentrated under reduced pressure to obtained crude compound. The crude compound was purified by CombiFlash column chromatography using 5% MeOH in DCM to afford 2-amino-N-(2,2-diethoxyethyl)-4-methyl-N-(2-methylbutyl)pentanamide (3.60 g, 72% yield) as a yellow viscous liquid. MS (ESI) m/z [M+H-EtOH]⁺: 272.10.

Step 3: Synthesis of (9H-fluoren-9-yl)methyl (3-((1-((2,2-diethoxyethyl)(2-methylbutyl)amino)-4-methyl-1-oxopentan-2-yl)amino)-3-oxopropyl)carbamate. To a stirred solution of 3-((((9H-fluoren-9-yl)methoxy)carbonyl)amino)propanoic acid (3.80 g, 12.28 mmol) in dry DMF (35 mL) maintained at 0° C. was added HATU (6.48 g, 17.05 mmol) and DIPEA (4.90 mL, 28.42 mmol), followed by the addition of 2-amino-N-(2,2-diethoxyethyl)-4-methyl-N-(2-methylbutyl)pentanamide (3.60 g, 11.37 mmol). The reaction mixture was allowed to attain room temperature and stirred for 3 h. After completion, the reaction mixture was quenched with ice cold water (20 mL) and the aqueous layer was extracted with EtOAc (30 mL×2). The organic layer was washed with cold H₂O (10 mL) followed by saturated brine (20 mL), dried over Na₂SO₄ and concentrated under reduced pressure. The crude compound was purified by Combiflash column chromatography using 5% MeOH in DCM to afford (9H-fluoren-9-yl)methyl (3-((1-((2,2-diethoxyethyl)(2-methylbutyl)amino)-4-methyl-1-oxopentan-2-yl)amino)-3-oxopropyl)carbamate (3.8 g, 55% yield) as an off-white solid. MS (ESI) m/z [M+H-EtOH]⁺: 565.30.

Step 4: Synthesis of (9H-fluoren-9-yl)methyl 6-isobutyl-8-(2-methylbutyl)-4,7-dioxohexahydro-2H-pyrazino[1,2-a]pyrimidine-1(6H)-carboxylate. To a stirred solution of (9H-fluoren-9-yl)methyl 6-isobutyl-8-(2-methylbutyl)-4,7-dioxohexahydro-2H-pyrazino[1,2-a]pyrimidine-1(6H)-carboxylate (3.80 g, 6.231 mmol) was added formic acid (20 mL) at room temperature and the reaction mixture was stirred for 16 h. After completion, the reaction mixture was concentrated under reduced pressure. The crude compound was purified by column chromatography (Silica 100-200 mesh; 0-5% MeOH in DCM) to afford (9H-fluoren-9-yl)methyl 6-isobutyl-8-(2-methylbutyl)-4,7-dioxohexahydro-2H-pyrazino[1,2-a]pyrimidine-1(6H)-carboxylate (3.60 g, 94% yield) as a yellow solid. MS (ESI) m/z [M+H]⁺: 518.23.

Step 5: Synthesis of 6-isobutyl-8-(2-methylbutyl)hexahydro-4H-pyrazino[1,2-a]pyrimidine-4,7(6H)-dione. To a solution of (9H-fluoren-9-yl)methyl 6-isobutyl-8-(2-methylbutyl)-4,7-dioxohexahydro-2H-pyrazino[1,2-a]pyrimidine-1(6H)-carboxylate (3.60 g, 6.954 mmol) in CH₂C₁₂ (36 mL) was added diethylamine (6.8 mL, 69.54 mmol) and the reaction mixture was stirred at room temperature for 16 h. After complete consumption of the starting material (monitored by TLC), the reaction mixture was concentrated under reduced pressure and the crude product was purified by combiflash column chromatography using 10-50% ethyl acetate in n-hexane to afford 6-isobutyl-8-(2-methylbutyl)hexahydro-4H-pyrazino[1,2-a]pyrimidine-4,7(6H)-dione (1.20 g, 60% yield) as a white solid. MS (ESI) m/z [M+H]⁺: 296.10.

Step 6: Synthesis of 1-(4-(difluoromethoxy)benzyl)-6-isobutyl-8-(2-methylbutyl)hexahydro-4H-pyrazino[1,2-a]pyrimidine-4,7(6H)-dione. To a solution of 6-isobutyl-8-(2-methylbutyl)hexahydro-4H-pyrazino[1,2-a]pyrimidine-4,7(6H)-dione (0.170 g, 0.576 mmol) in DMF (5 mL) was added K₂CO₃ (0.159 g, 1.152 mmol) at 0° C., and the reaction mixture was stirred for 10 min. To the resulting reaction mixture was added 1-(bromomethyl)-4-(difluoromethoxy)benzene (0.150 g, 0.632 mmol) at room temperature and stirred for 3 h. After completion, the reaction mixture was quenched with ice cold water (200 mL) and the aqueous layer was extracted with EtOAc (20 mL×2). The organic layer was washed with cold H₂O (20 mL) followed by saturated brine (15 mL), dried over Na₂SO₄ and concentrated under reduced pressure. The resulting crude compound was purified by PREP HPLC to afford 1-(4-(difluoromethoxy)benzyl)-6-isobutyl-8-(2-methylbutyl)hexahydro-4H-pyrazino[1,2-a]pyrimidine-4,7(6H)-dione (0.103 g, 40% yield) as a white solid. MS (ESI) m/z [M+H]: 452.3. ¹H NMR (400 MHz, DMSO d₆) δ 7.42 (d, J=8.8 Hz, 2H), 7.14-7.24 (m, 3H), 5.0-5.10 (m, 1H), 4.50-4.60 (m, 1H), 3.90-4.00 (m, 2H), 3.60-3.70 (m, 1H), 3.02-3.40 (m, 4H), 2.70-2.85 (m, 2H), 2.0-2.10 (m, 1H), 1.50-1.70 (m, 4H), 1.20-1.35 (m, 1H), 1.0-1.10 (m, 1H), 0.70-0.98 (m, 12H).

### Example 1: Treating Long COVID

### A. Overview

### ATH-1017

ATH-1017 is administered by subcutaneous (SC) injection once-daily (OD) preferably during daytime to a patient in need of treatment for long COVID. The first SC injection of ATH-1017 is performed at site under supervision.

Exemplary are pre-filled syringes of ATH-1017 at 40 mg contain 1.0 mL of 40 mg/mL ATH-1017 in a solution of 10 mM sodium phosphate and 0.5% NaCl. Pre-filled syringes of ATH-1017 at 70 mg contain 1.0 mL of 70 mg/mL ATH-1017 in a solution of 10 mM sodium phosphate. Each pre-filled syringe of placebo contains 1.0 mL of a solution of 10 mM sodium phosphate and 1.1% NaCl. All solutions are adjusted to pH of between about 7.0 and about 7.6.

ATH-1017 and placebo are administered subcutaneously.

### Compound of Formula (I) or a Pharmaceutically Acceptable Salt

A Compound of Formula (I), such as Compound 2a or Compound 1a, or a pharmaceutically acceptable salt thereof, is administered orally to a patient in need of treatment for long COVID.

### B. Screening Assessments

### Alzheimer's Disease Cooperative Study—Activities of Daily Living, 23-Item Version (ADCS-ADL23)

In some embodiments, a patient in need of treatment for long COVID is assessed with ADCS-ADL23. The ADCS-ADL23 (Galasko, 1997) is a 23-item assessment of functional impairment in terms of activities of daily living administered to the support person/caregiver. It comprises 23 questions about the subject's involvement and level of performance across items representing daily living. The questions range from basic to instrumental activities of daily living. Each item is rated from the highest level of independent performance to complete loss. The total score range is from 0 to 78, with lower scores indicating greater functional impairment.

### Alzheimer's Disease Cooperative Study—Activities of Daily Living, 19-Item Version (ADCS-ADL19)

In some embodiments, a patient in need of treatment for long COVID is assessed with ADCS-ADL19. The ADCS-ADL19 (Galasko, 1997) is a 19-item assessment of functional impairment in terms of activities of daily living administered to the support person/caregiver. It comprises 19 questions about the subject's involvement and level of performance across items representing daily living. The questions range from basic to instrumental activities of daily living. Each item is rated from the highest level of independent performance to complete loss. The total score range is from 0 to 54, with lower scores indicating greater functional impairment.

### Financial Capacity Instrument (FCI)

In some embodiments, a patient in need of treatment for long COVID is assessed with FCI. The financial Capacity Instrument (FCI) (Marson, 2000) is a prototype psychometric instrument that tests financial capacity using 14 tasks of financial ability comprising six clinically relevant domains of financial activity: bank statement management, basic monetary skills, cash transactions, checkbook management, financial conceptual knowledge, and financial judgment. Its usefulness is its sensitivity in very early stages of cognitive impairment.

### Mini-Mental State Examination (AMISE)

In some embodiments, a patient in need of treatment for long COVID is assessed with MMSE. The Mini-Mental State Examination (MMSE) (Folstein, 1975) is a widely used test of overall cognitive function, assessing memory, orientation and praxis in a short series of tests. The score is from 0 to 30 with 30 being the best possible and 0 being the worst possible score. The MMSE is used as a screening instrument to determine severity of cognitive impairment, with scores of 26 or less being by convention considered pathological.

### Alzheimer's Disease Assessment Scale—Cognitive Subscale (ADAS-Cog₁₁)

In some embodiments, a patient in need of treatment for long COVID is assessed with ADAS-Cog₁₁. The original ADAS-Cog₁₁ was designed to measure incremental changes in cognitive impairment in subjects with AD (Rosen, 1984), but has been used in other conditions of impaired cognition as well. The cognitive areas covered can be summarized as memory and new learning, language, and praxis. The standard 11 items are word recall, commands, constructional praxis, naming objects and fingers, ideational praxis, orientation, word recognition, spoken language ability, comprehension of spoken language, word-finding difficulty, and remembering test instructions. The test includes 7 performance items and 4 clinician-rated items, with a total score ranging from 0 (no impairment) to 70 (severe impairment). Therefore, higher scores indicate more severe cognitive impairment. Later versions (ADAS-Cog13, 14) added test items to increase sensitivity in less impaired cases.

Due to known circadian fluctuations of cognitive capacity (Hilt, 2015), ADAS-Cog₁₁ is assessed in the morning at approximately the same time of day as the baseline assessment to reduce variance in all subsequent tests.

### Montreal Cognitive Assessment Test (MoCA)

In some embodiments, a patient in need of treatment for long COVID is assessed with MoCA. The MOCA was designed as a rapid screening instrument for mild cognitive dysfunction. It assesses different cognitive domains: attention and concentration, executive functions, memory, language, visuoconstructional skills, conceptual thinking, calculations, and orientation. See Nasreddine Z S, et al. The Montreal Cognitive Assessment, MoCA: a brief screening tool for mild cognitive impairment. J Am Geriatr Soc. 2005 April; 53(4):695-9. Erratum in: J Am Geriatr Soc. 2019 Sep; 67(9):1991). The total possible score is 30 points; a final total score of 26 and above is considered normal.

### Digit Symbol Substitution Test (DSST)

In some embodiments, a patient in need of treatment for long COVID is assessed with DSST. The DSST is a timed neuropsychological test sensitive to brain damage, dementia, age and depression. See Jaeger, J. J Clin Psychopharmacol. 2018 October; 38(5): 513-519. The test is not sensitive to the location of brain-damage (except for damage comprising part of the visual field). It consists of (e.g. nine) digit-symbol pairs (e.g. 1/-, 2/⊥ . . . 7/Λ, 8/X, 9/=) followed by a list of digits. Under each digit the subject should write down the corresponding symbol as fast as possible. The number of correct symbols within the allowed time (e.g. 90 or 120 sec) is measured. The test procedure is language-free and patient results indicate unspecific cognitive performance impairment with high sensitivity.

### Disability Assessment for Dementia (DAD)

In some embodiments, patient in need of treatment for long COVID is assessed with DAD. The objectives of the DAD Scale are to quantitatively measure functional abilities in activities of daily living (ADL) in individuals with cognitive impairments such as dementia and to help delineate areas of cognitive deficits which may impair performance in ADL. See Gélinas I, et al. Development of a functional measure for persons with Alzheimer's disease: the disability assessment for dementia. Am J Occup Ther. 1999 Sep-Oct; 53(5):471-81. Basic and instrumental activities of daily living are examined in relation to executive skills to permit identification of the problematic areas and inform treatment decisions. Therefore, the activities of daily living have been subdivided and are assessed according to executive functions which have showed regression patterns in dementias. These are initiation, planning and organization, and effective performance.

### Neuropsychiatric Inventory (NPI)

In some embodiments, a patient in need of treatment for long COVID is assessed with NPI. The NPI is an interview-based rating scale of psychiatric and behavioral disturbances associated with dementia (Cummings, 1994). The support person/caregiver is interviewed by the qualified NPI rater about the presence or absence of 12 symptoms, including delusions, hallucinations, agitation/aggression, depression/dysphoria, anxiety, elation/euphoria, apathy/indifference, disinhibition, irritability/lability, aberrant motor behavior, sleep and nighttime behavior disorders, and appetite and eating disorders. For those symptoms present, the support person/caregiver rates the frequency, severity, and distress of each symptom. A total score and a caregiver distress score are calculated. A higher score indicates more severe psychopathology.

### Controlled Oral Word Association Test (COWAT)

In some embodiments, a patient in need of treatment for long COVID is assessed with COWAT, which is applied to assess executive memory function. The Controlled Oral Word Association Test (COWAT) is a timed oral verbal fluency test in which the subject is required to make verbal associations to different letters of the alphabet by saying all the words which they can think of beginning with a given letter. Individuals are given 1 minute to name as many words as possible beginning with each of the letters. The procedure is then repeated for the remaining two letters (Benton, 1994; Strauss, 2006). The test score is the total number of different words produced for all 3 letters.

### ERP P300

A patient in need of treatment for long COVID is assessed with ERP P300. ERP P300 is a quantitative, unbiased, non-invasive method of recording task-related brain processing speed, elicited by external stimuli, e.g., an oddball auditory stimulus, and is a well-established functional biomarker, particularly of working memory access (Ally, 2006). ERP P300 is characterized by a stereotyped series of voltage deflections occurring after the respective odd tone to be counted, with early features (<100 msec) corresponding to unconscious sensory transmission (auditory cortex, N100), and later features produced by cognitive processing in the ventral attentional network, i.e., P300, referring to the large positive deflection at roughly 300 msec in healthy adults (young or elderly). The P300 latency is sensitive to detecting reduced synaptic transmission related to cognitive decline in AD patients and other dementias (Olichney, 2011).

To assess the P300 wave (latency and amplitude), the subject has to perform a task related to auditory stimuli. The stimulus can, for example, consist of an oddball paradigm with 2 sound stimuli. Stimuli are presented through headphones and auditory stimulation for P300 is assessed in repeat recordings lasting in total up to 10 minutes.

### C. Results

Treatment with ATH-1017, Compound 2a or a pharmaceutically acceptable salt thereof, or Compound 1a or a pharmaceutically acceptable salt thereof, reduces the rate of decline, stabilizes, or improves patient outcome as measured by one or more of the above screening assessments, while having acceptable safety and tolerability.

### Example B2: Neuroprotection Against LPS Toxic Inflammatory Challenge

To assess the neuroprotective effect of compound treatment against inflammatory insult, such as that associated with long COVID, primary cultured cortical neurons were subjected to lipopolysaccharide (LPS) treatment for 24 h along with various compound doses of compounds of Formula (I) or compound A19. LPS is a potent activator of toll-like receptor 4 (TLR4), which is expressed on both neurons and microglia, and can initiate the release of several pro-inflammatory mediators. For example, LPS induces strong release of tumor necrosis factor-α (TNFα) and IL-6 in primary mouse neuronal cultures, both of which are hallmark pro-inflammatory mediators associated with neurodegeneration and cytotoxicity. To demonstrate the neuroprotective potential of the test compounds against inflammation-induced cytotoxicity, a Cell Titer-Glo Luminescent Cell Viability Assay (Promega, Cat #G7571) was utilized. Rat primary cortical neuron cultures were grown at 37° C., 5% CO₂ in Complete Neurobasal Medium supplemented with B27/GDNF/BDNF containing 10% FBS until maturity. 25 μl of culture volume were seeded in a 384-well plate at 5,000 cells/well, and treated with 1 μM, 100 nM, 10 nM, 1 nM, or 0.1 nM of test compound for 15 minutes. Cells were then subjected to 1 μM LPS challenge for 24 hours. Luminescence measurements to assess relative levels of ATP were performed on an Envision plate reader (Perkin Elmer). Data for DMSO control were set to 100% cell viability, and each group (n=4) were averaged and presented as mean±SEM.

The results are shown in Table 2. Compounds 1a, 2a, 5a, 6a, and A19 active metabolite all protected primary neurons from LPS-stimulated inflammatory toxicity at at least one dose tested. Statistical analyses were performed using one-way ANOVA with Tukey's Multiple comparison test. ++ indicates p<0.01 and +++ indicates p<0.001 against DMSO+LPS.

TABLE 2
Neuroprotection of rat cortical neurons against LPS challenge

| | | Significance of |
|---|---|---|
| | | neuroprotection vs 1 |
| Treatment | Dose | uM LPS |
| A19, active | 1 μM | ++ |
| metabolite | 100 nM | ++ |
| | 10 nM | +++ |
| | 1 nM | ++ |
| | 0.1 nM | +++ |
| Compound la | 1 μM | +++ |
| | 100 nM | +++ |
| | 10 nM | +++ |
| | 1 nM | +++ |
| | 0.1 nM | +++ |
| Compound 2a | 1 μM | +++ |
| | 100 nM | +++ |
| | 10 nM | +++ |
| | 1 nM | +++ |
| | 0.1 nM | +++ |
| Compound 5a | 1 μM | +++ |
| | 100 nM | +++ |
| | 10 nM | +++ |
| | 1 nM | +++ |
| | 0.1 nM | +++ |
| Compound 6a | 1 μM | +++ |
| | 100 nM | +++ |
| | 10 nM | +++ |
| | 1 nM | +++ |
| | 0.1 nM | +++ |

### Example B3: Neurotrophic Effects on Neurite Outgrowth and Synaptogenesis

Neurological disease progression is associated with a loss of neural network connectivity, which can have a variety of causes, including infection with SARS-CoV-2. Loss of intact brain circuitry is a primary driver of cognitive impairment, regardless of the source of the degenerative cause. Neural connectivity is driven by extension of axonal or dendritic processes, collectively termed dendrites, and the formation of functional chemical connections between neurons, collectively termed synapses. The ability of test compounds to stimulate neurons to extend neurite processes and form synapses in culture was tested.

Rat hippocampal neurons from newborn pups were isolated and cultured in brain neuronal culture medium (neurobasal medium supplemented with 5% FBS, 2% B-27 supplement, and 0.003% gentamycin) at 37° C. under 5% CO₂. At 1 day in vitro (DIV1), cells were transferred to a poly-D-lysine-coated 24-well plate at approximately 10,000 cells/well. Cells were then grown in 3 mL of serum-free medium and treated with the following: 0.1% dimethyl sulfoxide (DMSO; vehicle), 10 nM dihydrotestosterone (positive control), or Compound A19 active metabolite at the following concentrations: 0, 1, 1, or 10 nM. Compound A19 is a prodrug, therefore when testing this compound in vitro it is more appropriate to administer the active metabolite form. All treatments contained a final concentration of 0.1% DMSO. For the neurite outgrowth assay, treatment media were refreshed on DIV3, and cells were processed for analysis on DIV4. For the synaptogenesis assay, treatment media were refreshed on DIV3 and DIV6, and cells were processed for analysis on DIV8. Processing for both the neurite outgrowth and the synaptogenesis assays was performed as follows: media were removed, and cells were fixed with 100% methanol for 20 minutes at room temperature (RT) before methanol was aspirated and each well was washed twice with Dulbecco's phosphate-buffered saline (DPBS). Cells were permeabilized using 0.02% Triton X-1000 (ThermoFisher, Waltham, Massachusetts) in DPBS for 2 minutes at RT without shaking, and plates were blocked with 5% bovine serum albumin (BSA) in DPBS for 1 hour at RT. Plates were then incubated overnight at 4° C. with gentle shaking with beta-III tubulin antibody (ab201740; Abcam, Boston, Massachusetts) to assess neurite outgrowth or with 1:500 rabbit anti-synaptobrevin-II antibody (Synaptic Systems, cat #104 202, Göttingen, Germany) to assess synaptogenesis. After overnight incubation, plates were washed 3 times with DPBS and incubated for 1 hour at RT with anti-rabbit immunoglobulin G-secondary antibody conjugated to Alexa Fluor 488 (Life Technologies, Carlsbad, California, 1:500).

Imaging was performed using the BX43 Olympus microscope driven by the standard “CellSens” software by Olympus. Images were taken under 60×water-dipping objective using a DP74 camera (Olympus, Center Valley, Pennsylvania). Ten images of single cells from a total of 2 assays were analyzed using NeuroJ plug-in for ImageJ to assess neurite length. For the synaptogenesis assay, synaptic counts (number of synapses) were quantified for each cell. Data were assessed for normality (Shapiro-Wilk) and homoscedasticity (Brown-Forsythe). For data analysis, the average of each treatment group was compared with the average of the vehicle (0.1% DMSO) group and analyzed using 1-way ANOVA/Dunnett's posttest. A value of p<0.05 was considered to represent a statistically significant difference.

The results are shown in Table 3. Treatment with compound A19 active metabolite promoted the extension of neurites in this study.

TABLE 3
Compound A19 active metabolite promotes extension
of neurites in primary neurons

| | | Significant increase in: | |
|---|---|---|---|
| Treatment | Dose | Neurite length | Synapses |
| DHT | 10 nM | + | NS |
| Cmpd A19 active | 0.1 nM | NS | NS |
| metabolite | 1 nM | NS | NS |
| | 10 nM | ++++ | NS |

DHT = alpha-dihydrotestosterone, positive control compound.
Cpmd = Compound.
Statistics applied: 1-way ANOVA with Dunnett's multiple comparison's post-test compared to vehicle control.
NS = not significant, + = p < 0.05, ++++ = p < 0.0001.

An identical study was carried out to test the ability of additional test compounds to stimulate neurite outgrowth and synapse formation. Compounds 1a, 2a, and 5a also demonstrated the ability to increase the length of neurites and number of synapses in cultured primary neurons. In this study, Compound 1a was found to increase both measures at all doses tested. Compound 2a was found to increase neurite length at a concentration of 10 nM and increase synapse counts at all concentrations tested. Compound 5a was found to increase neurite length at 1 nM and increase synapse counts at doses of 1 nM and 100 nM (Table 4). HGF at a dose of 5 ng/ml was also used as a positive control and was found to increase both neurite length and synapse count. These results are shown in Table 4.

TABLE 4
Compound la, Compound 2a, and Compound 5a promote extension
of neurites and formation of synapses in primary neurons

| | | Significant increase in: | |
|---|---|---|---|
| Treatment | Dose | Neurite length | Synapses |
| DHT | 10 nM | + | +++ |
| HGF | 5 ng/ml | +++ | ++++ |
| Cmpd la | 1 nM | ++++ | ++++ |
| | 10 nM | ++++ | ++++ |
| | 100 nM | + | ++++ |
| Cmpd 2a | 1 nM | NS | ++++ |
| | 10 nM | ++++ | ++++ |
| | 100 nM | NS | ++++ |
| Cmpd 5a | 1 nM | ++++ | ++++ |
| | 10 nM | NS | NS |
| | 100 nM | NS | ++++ |

DHT = alpha-dihydrotestosterone, positive control compound, HGF = hepatocyte growth factor.
Statistics applied: 1-way ANOVA with Dunnett's multiple comparison's post-test compared to vehicle control.
NS = not significant, + = p < 0.05, +++ = p < 0.0005 ++++ = p < 0.0001.

### Example B4: Rodent Lipopolysaccharide Model of Brain Inflammation

Inflammation of the brain (encephalitis) that may result in brain swelling fever, headache, confusion, and seizures. The source of the neuroinflammation can be widely varied, including due to viral, bacterial, parasitic, or fungal infection or due to autoimmune diseases. Neuroinflammation has been reported in cases of long COVID. Lipopolysaccharide (LPS) is a bacterial endotoxin that induces an intense neuroinflammatory reaction and has been associated with cognitive decline. The ability of Compound A19 to reverse cognitive decline induced by LPS in a mouse model of encephalitis was studied, using a T-maze assay. In a separate identical assay, Compounds 1a and 5a were also assessed.

Spontaneous and continuous alternation is the innate tendency of rodents to alternate free choices in a T-maze (T-CAT) over a series of successive runs. This sequential procedure relies on working memory and is sensitive to various pharmacological manipulations affecting memory processes. The present studies were conducted to evaluate the ability of Compound A19, Compound 1a, and Compound 5a to reverse LPS-induced cognitive deficit in the T-maze assay.

Compound A19 was dissolved in 0.9% NaCl (saline) and vortexed periodically until complete dissolution and with pH adjusted between 7.4 and 7.8. Compound A19 was used in concentrations of 0.125, 0.1, 0.05, 0.025 and 0.0125 mg/ml which when given in a volume of 10 ml/kg result in doses of 1.25, 1, 0.5, 0.25 and 0.125 mg/kg, respectively. Compound 1a was dissolved at 160 mg/ml in 100% DMSO and vortexed until complete dissolution. Then the above stock solution was diluted in 20% PEG-400, and 78% 0.9% NaCl whereby the stock solution comprised 2% of the final solution. Compound 1a was used in concentrations of 0.05, 0.2, 0.8, 1.6 and 3.2 mg/ml which when given in a volume of 10 ml/kg result in doses of 0.5, 2, 8, 16 and 32 mg/kg, respectively. Compound 5a was dissolved at 100 mg/ml in 100% DMSO and vortexed until complete dissolution. Then the above stock solution was diluted in 20% PEG-400, and 78% 0.9% NaCl whereby the stock solution comprised 2% of the final solution. Compound 5a was used in concentrations of 0.125, 0.25, 0.5, 1 and 2 mg/ml which when given in a volume of 10 ml/kg result in doses of 1.25, 2.5, 5, 10 and 20 mg/kg, respectively.

Memantine was dissolved in saline. Memantine was used in concentration of 0.1 and 0.01 mg/ml, which when given in a volume of 10 ml/kg result in a dose of 1 and 0.1 mg/kg, respectively. Memantine is approved for symptomatic treatment of Alzheimer's disease was tested as a reference compound.

LPS was dissolved in saline and prepared at a concentration of 0.0125 mg/ml given i.p. at a dosage volume of 20 ml/kg to yield a dose of 0.25 mg/kg (250 μg/kg).

Four to five weeks old male CD-1 mice (Janvier; Le Genest St Isle-France) were used for the study.

Compound A19 was tested at the doses of 1.25, 1, 0.5, 0.25 and 0.125 mg/kg administrated subcutaneously (s.c.) for 14 days with last treatment given 60 min before the T-maze trial. Compound 1a was tested at the doses of 0.5, 2, 8, 16, and 32 mg/kg administered orally (p.o.) for 14 days with last treatment given 60 min before the T-maze trial. Compound 5a was tested at the doses of 1.25, 2.5, 5, 10, and 20 mg/kg administered orally (p.o.) for 14 days with last treatment given 60 min before the T-maze trial. LPS was injected i.p. 2 weeks prior the T-maze trial. Table 5 shows the administration route and treatment schedule for each treatment group.

TABLE 5
Treatment Schedule

| Group No. | Description | route | Treatment & Time before trial (min) |
|---|---|---|---|
| 1 | Saline/Vehicle | ip/sc | Once daily from the LPS |
| 2 | LPS/Vehicle | ip/sc | administration for 14 days. Last |
| | | | treatment 60 min before T-maze |
| 3 | LPS/Memantine 0.1 mg/kg | ip/ip | Once daily from the LPS |
| 4 | LPS/Memantine 1 mg/kg | ip/ip | administration for 14 days. Last |
| | | | treatment 60 min before T-maze |
| 5 | LPS/A19 1.25 mg/kg | ip/sc | Once daily from the LPS |
| 6 | LPS/A19 1 mg/kg | ip/sc | administration for 14 days. Last |
| 7 | LPS/A19 0.5 mg/kg | ip/sc | treatment 60 min before T-maze |
| 8 | LPS/A19 0.25 mg/kg | ip/sc | |
| 9 | LPS/A19 0.125 mg/kg | ip/sc | |
| 10 | LPS/Compound la 0.5 mg/kg | ip/po | |
| 11 | LPS/Compound la 2 mg/kg | ip/po | |
| 12 | LPS/Compound la 8 mg/kg | ip/po | |
| 13 | LPS/Compound la 16 mg/kg | ip/po | |
| 14 | LPS/Compound la 32 mg/kg | ip/po | |
| 15 | LPS/Compound 5a 1.25 mg/kg | ip/po | |
| 16 | LPS/Compound 5a 2.5 mg/kg | ip/po | |
| 17 | LPS/Compound 5a 5 mg/kg | ip/po | |
| 18 | LPS/Compound 5a 10 mg/kg | ip/po | |
| 19 | LPS/Compound 5a 20 mg/kg | ip/po | |

The T-maze apparatus was made of gray Plexiglas with a main stem (55 cm long×10 cm wide×20 cm high) and two arms (30 cm long×10 cm wide×20 cm high) positioned at a 90-degree angle relative to the main stem. A start box (15 cm long×10 cm wide) was separated from the main stem by a guillotine door. Horizontal doors were also provided to close specific arms during the force choice alternation task.

The experimental protocol consisted of a single session, which started with one “forced-choice” trial, followed by 14 “free-choice” trials. In the “forced-choice” trial, the animal was confined 5 seconds in the start arm and was then released while either the left or right goal arm was blocked by a horizontal door. It negotiated the maze but eventually it entered the open goal arm and returned to the start position. Immediately after the animal returned to the start position; the left or right goal door was opened and the animal was allowed to choose freely between the left and right goal arm (“free choice trials”). The animal was considered have entered into an arm when it placed its four paws in the arm. A session was terminated, and the animal was removed from the maze as soon as 14 free-choice trials had been performed or 10 min had elapsed, whichever event occurred first.

The percentage of alternation (number of alternation/14*100) over the 14 free-choice trials was determined for each mouse and was used as an index of working memory performance. This percentage was defined as entry in a different arm of the T-maze over successive trials (i.e. left-right-left-right. etc).

Analysis of variance (ANOVA) was performed on the result data. Fisher's PLSD test was used for pairwise comparisons and p value≤0.05 were considered significant. The drug-induced reversion of LPS-induced memory deficit was calculated by setting the respective response of the saline/vehicle as 100% and that of the group LPS/vehicle as 0% reversion.

A single injection of 250 μg/kg LPS resulted in a significant reduction of spontaneous alternation of mice (about 24% reduction in the study assessing A19 and 44% reduction in the study assessing Compound 1a and Compound 5a) as compared to the performance of saline-injected mice (LPS-free) when assessed 2 weeks following injection. Indeed, in the study assessing A19 the alternation rate was 45 and 69% for LPS/Vehicle and Saline/Vehicle groups, respectively. In the study assessing Compounds 1a and 5a, the alternation rate was 44% and 70% for the LPS/Vehicle and Saline/Vehicle groups, respectively. The reduction in the alternation rate of LPS-mice suggests a cognitive deficit. Table 6 shows the spontaneous alternation and recovery percentage for each treatment group.

As shown in Table 6, Compound A19 produced a dose-dependent reversion of the LPS-induced deficit in the mouse T-maze alternation test. Indeed, the alternation rate was 61, 61, 64, 60 and 42% for the doses of 1.25, 1, 0.5, 0.25 and 0.125 mg/kg Compound A19, respectively. These alternation rates correspond to 65, 65, 79, 62 and −12% recovery in the cognitive performance of LPS-mice for the doses of 1.25, 1, 0.5, 0.25 and 0.125 mg/kg Compound A19, respectively. The effect of Compound A19 was significant at all doses except at 0.125 mg/kg. Additionally, Compound 1a produced a dose-dependent reversion of the LPS-induced deficit in the mouse T-maze alternation test. The alternation rate was 49, 59, 56, 51, and 56% for 0.5, 2, 8, 16 and 32 mg/kg, respectively. The reversal rate was 17, 56, 47, 28 and 44% for 0.5, 2, 8, 16 and 32 mg/kg, respectively. Compound 5a was also assessed for reversal of LPS-induced deficits in the mouse T-maze alternation test. The alternation rate was 59, 58, 61, 58, and 60% for 1.25, 2.5, 5, 10, and 20 mg/kg, respectively. The reversal rate was 58, 53, 64, 53 and 61% for 1.25, 2.5, 5, 10 and 20 mg/kg, respectively.

LPS-mice treated with memantine (1 and 0.1 mg/kg) showed 54 and 64% alternation rate, respectively (see Table 6). These rates correspond to 38 and 76% recovery in the cognitive performance of LPS treated mice. The effect of Memantine was significant at both doses.

In line with literature findings, memantine produced a significant increase in the spontaneous alternation of LPS-treated mice, which suggests an improved cognitive performance in the T-maze assay. While 0.125 mg/kg Compound A19 (the lowest tested dose) was ineffective in reversing LPS-induced cognitive deficit, higher dose levels (up to 1.25 mg/kg) significantly improved the cognitive performance of LPS-mice.

The results for Compounds 1a and 5a are shown in Table 7. Compound 1a exhibited significant reversal of LPS-induced cognitive deficits at 2 mg/kg, and was trending at 8 and 32 mg/kg. Compound 5a resulted in significant reversal of LPS-induced cognitive deficits at all doses.

The results indicate that Compound A19, Compound 1a, and Compound 5a reversed the LPS-induced cognitive disruption in the mouse T-maze assay.

TABLE 6
Spontaneous Alternation and Recovery Percentage

| | Spontaneous Alternation (%) | | | |
|---|---|---|---|---|
| Group Names | Mean | s.e.m. | n | Recovery (%) |
| Vehicle/Saline | 69.3 | 2.1 | 10 | 100 |
| Vehicle/LPS 250 μg/kg | 45.0 | 2.1 | 10 | 0 |
| A19 1.25 mg/kg/LPS 250 μg/kg | 60.7 | 1.9 | 10 | 58 |
| A19 1 mg/kg/LPS 250 μg/kg | 60.7 | 2.4 | 10 | 65 |
| A19 0.5 mg/kg/LPS 250 μg/kg | 64.3 | 3.8 | 10 | 79 |
| A19 0.25 mg/kg/LPS 250 μg/kg | 60.0 | 3.0 | 10 | 62 |
| A19 0.125 mg/kg/LPS 250 μg/kg | 42.1 | 2.5 | 10 | −12 |
| Memantine 1 mg/kg/LPS 250 μg/kg | 54.3 | 3.2 | 10 | 38 |
| Memantine 0.1 mg/kg/LPS 250 μg/kg | 63.6 | 4.3 | 10 | 76 |

TABLE 7
Spontaneous Alternation and Recovery percentage

| | Spontaneous alternation | | | |
|---|---|---|---|---|
| | (%) | | | Recovery |
| Group names | Mean | s.e.m. | n | (%) |
| Vehicle/Saline | 70.0 | 2.3 | 10.0 | 100 |
| Vehicle/LPS 250 μg/kg | 44.3 | 3.0 | 10.0 | 0 |
| Cmpd la 0.5 mg/kg/LPS 250 μg/kg | 48.6 | 5.8 | 10.0 | 17 |
| Cmpd la 2 mg/kg/LPS 250 μg/kg | 58.6 | 2.8 | 10.0 | 56 |
| Cmpd la 8 mg/kg/LPS 250 μg/kg | 56.4 | 4.0 | 10.0 | 47 |
| Cmpd la 16 mg/kg/LPS 250 μg/kg | 51.4 | 1.8 | 10.0 | 28 |
| Cmpd la 32 mg/kg/LPS 250 μg/kg | 55.7 | 2.6 | 10.0 | 44 |
| Cmpd 5a 1.25 mg/kg/LPS 250 | 59.3 | 3.0 | 10.0 | 58 |
| μg/kg | | | | |
| Cmpd 5a 2.5 mg/kg/LPS 250 μg/kg | 57.9 | 2.7 | 10.0 | 53 |
| Cmpd 5a 5 mg/kg/LPS 250 μg/kg | 60.7 | 2.7 | 10.0 | 64 |
| Cmpd 5a 10 mg/kg/LPS 250 μg/kg | 57.9 | 2.9 | 10.0 | 53 |
| Cmpd 5a 20 mg/kg/LPS 250 μg/kg | 60.0 | 2.4 | 10.0 | 61 |

### Example B5: Reduction of Pro-Inflammatory Cytokine Expression in Macrophage-Like Cell Cultures

Neuroinflammation plays a crucial role in disease progression, such as long COVID, and is a complex phenomenon involving various cells that affect many extra- and intracellular signaling pathways and cytokine production. The macrophage of the brain (microglia) is the main agent of innate immunity in the CNS and perform various functions, including neuroprotection, in response to inflammatory signals. Activated microglia participate in developing neuroinflammation, responding to toxic exogenous substances (like LPS) or endogenous substances (like amyloid-beta). Here, we use THP1 cells as an in-vitro cell model to evaluate mechanisms relevant to CNS inflammation. Differentiation with PMA causes THP1 monocyte cells to acquire functional and morphological resemblance to macrophages. LPS interacts with THP-1 differentiated macrophage through the toll-like receptor 4, triggering the inflammatory response and stimulating pro-inflammatory cytokine release and eventually leading to cellular death. Here we investigated whether test compounds have anti-inflammatory effects on LPS-challenged macrophage cultures. THP1-differentiated macrophages were treated with test compounds for 20 minutes, followed by 24 hrs of LPS challenge. Culture supernatants were then collected to analyzed for the presence of pro-inflammatory cytokines: interleukin 1β (IL-1β), interleukin 6 (IL-6) and tumor necrosis factor α (TNF-α).

Cytokine quantification in cell culture supernatants was accomplished by HTRF kits to assess levels of IL-1β (Human IL-1β kit, #62HIL1BPEG, Cisbio) and IL-6 (Human IL-6 kit, 62HIL06PET, Cisbio), and by ELISA (Human TNF-α ELISA kit, KHC3011, ThermoFisher) to determine the levels of TNF-α.

Data analysis was performed via 1-way ANOVA with Tukey post-test compared to LPS treatment cultures using Prism statistical software (GraphPad). Tabular data indicates significance of reduction in culture supernatants for the indicated analyte at the indicated dose.

The results are collected in Table 8. Treatment with Compounds 1a, 5a, 6a, and Compound A19 active metabolite (dihexa) all significantly reduced the expression of IL-1β and TNF-α at at least one tested dose. Treatment with Compounds 1a, 5a, and A19 active metabolite all significantly reduced the expression of IL-6 at at least one tested dose “NS” indicates a non-significant reduction in the indicated cytokine. “+” indicates a p value<0.05 in Tukey post test. “++” indicates a p value<0.01 in Tukey post test.

TABLE 8
Significant reduction in pro-inflammatory cytokine
expression by THP-1 immune cells in vitro
Compound 1a

| Dose | Inflammatory Analyte | | |
|---|---|---|---|
| (nM) | IL-1β | IL-6 | TNF-α |
| 0.1 | ++ | NS | ++ |
| 10 | ++ | ++ | ++ |
| 1000 | ++ | + | ++ |

Compound 5a

| Dose | Inflammatory Analyte | | |
|---|---|---|---|
| (nM) | IL-1β | IL-6 | TNF-α |
| 0.1 | ++ | NS | NS |
| 10 | ++ | + | NS |
| 1000 | ++ | ++ | + |

Compound 6a

| Dose | Inflammatory Analyte | | |
|---|---|---|---|
| (nM) | IL-1β | IL-6 | TNF-α |
| 0.1 | NS | NS | NS |
| 10 | NS | NS | NS |
| 1000 | + | NS | + |

Compound A19, active metabolite

| Dose | Inflammatory Analyte | | |
|---|---|---|---|
| (nM) | IL-1β | IL-6 | TNF-α |
| 0.1 | NS | NS | NS |
| 10 | ++ | + | + |
| 1000 | + | ++ | ++ |

### Example B6: Treatment of Bleomycin-Induced Pulmonary Fibrosis with Compound 1a

The ability of Compound 1a to reduce pulmonary fibrosis was tested in a model of bleomycin-induced pulmonary fibrosis. Studies of some long COVID patients develop pulmonary fibrosis.

Briefly, Male C₅₇BL/6 mice (Envigo) with their tails removed were used. The mice were 3.5-4 months old and ˜24-30 g by weight. On day −1 animals were randomized based on body weight, and the animals were assigned with a permanent number by ear notching. Cages were identified by cage cards indicating the study code, group number, sex, dose, cage number, number of animals and animal number details.

On day 0, all animals (except the sham control animals) were administered bleomycin (0.03 U in 50 l normal saline) intratracheally (i.t.) under Ketamine+Xylazine anesthesia. The sham control animals were dosed with 50 l of normal saline intratracheally. Pirfenidone was used as a positive control; this compound is known to downregulate growth factors and procollagens to reduce fibrosis and is an approved drug for the treatment of idiopathic pulmonary fibrosis.

Vehicle, pirfenidone and test Compound 1a dosing was initiated from day 3 to day 21 as described in the following Table 9. On day 21 whole blood was collected 1-hour post-dose into EDTA and then processed to plasma. Right lungs were then collected, weighed, snap frozen and later homogenized for biochemical analysis. Hydroxyproline, collagen and TGF-Beta levels were measured in the right lung homogenates.

The study groups were as follows:

TABLE 9
Study Design

| | | Inducing Agent | Treatment | Dose | Number of |
|---|---|---|---|---|---|
| Group | Group Name | (i.t.) | Regimen | (mg/kg) | Animals |
| 1 | Sham control | 50 μl Normal | | NA | 10 |
| | | Saline | | | |
| 2 | BLM control | 0.03U BLM in | po, QD | NA | 10 |
| | | 50 μl saline | | | |
| 3 | Pirfenidone | 0.03U BLM in | po, BID | 100 | 10 |
| | | 50 μl saline | | | |
| 4 | Cmpd la | 0.03U BLM in | po, QD | 0.2 | 10 |
| | | 50 μl saline | | | |
| 5 | Cmpd la | 0.03U BLM in | po, QD | 0.8 | 10 |
| | | 50 μl saline | | | |
| 6 | Cmpd la | 0.03U BLM in | po, QD | 2 | 10 |
| | | 50 μl saline | | | |
| 7 | Cmpd la | 0.03U BLM in | po, QD | 8 | 10 |
| | | 50 μl saline | | | |

Abbreviations:
Cmpd la means Compound la;
BLM means bleomycin;
i.t. means intratracheally

Disease induction in this mouse model by bleomycin caused significant weight reduction, as determined by daily body weight monitoring. Treatment with Compound 1a or pirfenidone did not protect against body weight loss. Body weight loss was similar in the bleomycin alone, bleomycin plus pirfenidone and bleomycin plus Compound 1a groups over the study period. Mice treated with Compound 1a at 0.8 mg/k exhibited slightly greater weight loss as compared to the other treatment groups.

The weight of whole right lungs at termination (day 21) was less in the treatment groups using the highest doses of Compound 1a, 8 mg/kg and 2 mg/kg, and was less than the positive control, pirfenidone-treated animals. Because fibrosis increases lung weight, this result indicated that Compound 1a was active in reducing fibrosis in this model.

Collagen is deposited at the sites of injury and fibrosis in this model. Hydroxyproline concentration, a major component of protein collagen and a biomarker of fibrosis, was measured in right lung homogenates at termination. The treatment groups using the highest doses of Compound 1a, 8 mg/kg and 2 mg/kg, had lower concentrations of hydroxyproline; the hydroxyproline concentration in these groups was less than the positive control, pirfenidone-treated animals. Similarly, the collagen concentration in right lung homogenates at 8 mg/kg of Compound 1a was reduced compared to the pirfenidone positive control. Treatment with 2 mg/kg of Compound 1a and with pirfenidone resulted in about the same levels of collagen in right lung homogenate.

TGF-β promotes fibrosis in this model by increasing macrophage densities at the site of injury. At day 21, all doses of Compound 1a and of pirfenidone significantly protected against bleomycin-induced increases in TGF-β in right lung homogenates. Table 10 summarizes the changes in bleomycin-induced (BLM-induced) alterations in this model.

TABLE 10
Improvement in BLM-induced alterations versus disease control

| | Pirfenidone | Compound la | | | |
|---|---|---|---|---|---|
| Dose | 100 | 8 | 2 | 0.8 | 0.2 |
| Body Weight | | | | | |
| Right Lung | + | ++ | + | + | |
| Weight | | | | | |
| Hydroxyproline | | + | ++ | | |
| Collagen | ++ | ++- | ++ | + | |
| TGF-Beta | +++ | + | +++ | +++ | +++ |

+ => 5-25% improvement v disease control;
++ => 26-50% improvement v disease control;
+++ => 51-100% improvement v disease control

These data indicate that Compound 1a was active in treating pulmonary fibrosis in the model.

### Example B7: Treatment of Acute Pulmonary Inflammation as a Precursor to Pulmonary Fibrosis

Acute pulmonary inflammation can be effectively modeled in rats by intratracheal administration of bacterial lipopolysaccharides (LPS). Pulmonary inflammation that is left unresolved can lead to pulmonary fibrosis, which has been reported in long Covid patients. The normal process of inflammation is regulated and promoted by expression, secretion, and distribution of inflammatory signaling proteins. Therapeutics intended to reduce inflammation are commonly identified by their ability to change the expression levels of inflammatory signaling molecules, either by disruption of the inflammatory pathway directly, or resolution of the inflammatory signaling cascade. The severity of inflammatory response can be measured by quantifying the amount of immune cell infiltration and secretion of cytokine signaling proteins in bronchoalveolar lavage fluid (BALF). Activation of the HGF/MET signaling system by HGF/MET positive modulators is expected to reduce the inflammatory response. Two test compounds, Compound 1a and Compound 5a, were evaluated for their ability to reduce acute pulmonary inflammation in an LPS model.

In this study, acute pulmonary inflammation was induced in healthy male Sprague-Dawley rats (7-9 weeks old at study start) by intratracheal instillation of 20 g LPS in saline. Test animals were treated with test compounds twice, at 24 hours and 0.5 hours before LPS treatment by intravenous (IV) injection. The anti-inflammatory corticosteroid, Dexamethasone (Dex), was used as a positive control, and was administered 1 hour before LPS treatment by intraperitoneal (IP) injection at 3 mg/kg. Four hours post LPS administration, animals were euthanized by intraperitoneal injection of thiopentone and 20 mL of cold Hanks Balanced Salt Solution, pH 7.2 was infused into the lungs through tracheal cannulation. BALF was then collected.

Immune cell infiltration in BALF was quantified as an assessment of inflammatory response, with the results shown in Table 11. Immune cell infiltration was performed by counting total leukocytes using a MUSE® mini flow cytometer, and by manual counting of neutrophil cells in cytospin smears stained with Leishman's staining reagent. Treatment with Compound 1a and Compound 5a reduced both total leukocyte counts and neutrophil counts compared to LPS-only controls.

TABLE 11
Immune cell infiltration in BALF from
pulmonary LPS-stimulated rats

| | Dex | Compound 1a | | | Compound 5a | | |
|---|---|---|---|---|---|---|---|
| Dose (mg/kg) | 3 | 0.75 | 0.075 | 0.0075 | 7.5 | 0.75 | 0.075 |
| Total | +++ | | + | ++ | + | + | ++ |
| Leukocytes | | | | | | | |
| Neutrophils | +++ | | + | ++ | + | + | ++ |

+ = >5% reduction in cell count vs disease control
++ = >25% reduction in cell count vs disease control
+++ = >75% reduction in cell count vs disease control

Cytokine expression in BALF supernatant was quantified by multiplex immunoassay using a MAGPIX multiplexing unit with a cytokine multiplex kit (Merck-Millipore, Cat #RECYMAG65K27PMX). The panel of tested cytokines included growth factors (G-CSF, GM-CSF, EGF, VEGF, TNF-α), chemokines (CCL2, CCL3, CCL5, CCL11, CXCL 1, CXCL 2, CXCL5, CXCL10, CX3CL1), interleukins (IL-1α, IL-1β, IL-2, IL-4, IL-5, IL-6, IL-10, IL-12p70, IL-13, IL-17A), and interferon gamma (IFNγ). The results of this analysis are shown in Table 12. Statistically significant change from LPS-only animals was determined by Student's T-test, with p<0.05 indicating statistically significant change in cytokine expression levels. Treatment with Compound 1a lead to significant changes in expression of G-CSF, CXCL2, CXCL10, IFN-γ, and IL-1β. Treatment with Compound 5a lead to significant changes in expression of CCL3, CXCL1, IFN-γ, IL-1α, IL-1p, IL-1β, IL-12p70, and IL-17A. These results indicate that treatment with test compounds had a meaningful impact on acute pulmonary inflammation response to LPS by decreasing the levels of proinflammatory molecules. The normal process of inflammation is regulated and promoted by expression, secretion, and distribution of inflammatory signaling proteins.

TABLE 12
Significant change in cytokine expression in BALF
supernatant from pulmonary LPS-stimulated rats

| | | Dex | Compound 1a | | | Compound 5a | | |
|---|---|---|---|---|---|---|---|---|
| Dose (mg/kg) | | 3 | 0.75 | 0.075 | 0.0075 | 7.5 | 0.75 | 0.075 |
| Growth | G-CSF | + | + | | | | | |
| Factors | GM-CSF | | | | | | | |
| | EGF | + | | | | | | + |
| | VEGF | | | | | | | |
| | TNF-α | + | | | | | | |
| Chemokines | CCL2 | + | | | | | | |
| | CCL3 | + | | | | | | + |
| | CCL5 | + | | | | | | |
| | CCL11 | + | | | | | | |
| | CXCL1 | + | | | | | | |
| | CXCL2 | | + | | | | | |
| | CXCL5 | + | | | | | | |
| | CXCL10 | + | + | | + | | | |
| | CX3CL1 | + | | | | | | + |
| | IFN-γ | + | | | + | | + | + |
| Interleukins | IL-1α | + | | | | + | + | + |
| | IL-1β | + | + | | | | + | + |
| | IL-2 | | | | | | | |
| | IL-4 | | | | | | | |
| | IL-5 | + | | | | | | |
| | IL-6 | + | | | | | | |
| | IL-10 | + | | | | + | + | + |
| | IL-12p70 | + | | | | | | + |
| | IL-13 | + | | | | | | |
| | IL-17A | + | | | | | + | + |

+ = Significant change in cytokine quantification, p < 0.05 by Student's T-test.

### Example B8: In Vivo Efficacy: Scopolamine-Induced Spatial Memory Deficit in the Morris Water Maze

Compounds 2a and 6a were evaluated for their ability to reverse scopolamine-induced spatial memory deficits in rats in the Morris water maze. Scopolamine impairs cognition by inhibition of a neurotransmitter system, acetylcholine and muscarinic acetylcholine receptors. It has been suggested that the SARS-CoV-2 viral spike protein may bind to nicotinic acetylcholine receptors and produce cognitive defects by inhibiting that receptor. Compound A19 and the compounds of Formula 1 are proposed to be able to overcome inhibition of nicotinic acetylcholine receptors and thereby compensate for cognitive impairment. The activity of Compounds 2a and 6a was tested in this scopolamine-induced spatial memory deficit model to determine whether Compound A19 and the compounds of Formula 1 could compensate for cognitive defects such as spatial memory defects via a related neurotransmitter system.

The water maze consists of a large round tank (diameter 2.1 m) filled with 26-28° C. water to a depth of ˜30 cm and the water was clouded with white paint. A round platform (13 cm diameter) was fixed such that it rested 2-3 cm below the surface of the water. High-contrast visual cues were placed around the tank to aid spatial orientation of test animals. Testing consisted of placing an animal into the water facing the tank wall at one of three randomly assigned starting locations and allowing the animal to swim and search for the hidden platform for up to 120 seconds. The time taken for the animal to locate the platform was recorded as the escape latency. Animals were tested 5 times per day with a 30 second rest period between trials. Testing was completed for a total of 8 consecutive days.

Animals were divided into groups (N=8 per group) depending on treatment. Control animals received only empty vehicle. Scopolamine groups received 3 mg/kg scopolamine dissolved in sterile saline by intraperitoneal (IP) injection 30 minutes prior to testing. Test compound groups received test compound at various concentrations by oral gavage (PO) dissolved in 48% sterile saline, 50% polyethylene glycol (PEG-400), and 2% DMSO 40 minutes prior to testing. Escape latencies were recorded for each animal for 5 trials each day for 8 consecutive days. Changes in escape latency curves were statistically analyzed by 2-way ANOVA with Bonferoni post-test. Results are shown in Table 13.

Exemplary compound 1a was evaluated for its ability to reverse chemically-induced spatial memory deficits in rats in the Morris water maze. The water maze consists of a large round tank (diameter 1.5 m) filled with 23-26° C. water to a depth of ˜30 cm and the water was clouded with white paint. A round platform was fixed such that it rested 2-3 cm below the surface of the water. High-contrast visual cues were placed around the tank to aid spatial orientation of test animals. Testing consisted of placing an animal into the water facing the tank wall at one of three randomly assigned starting locations and allowing the animal to swim and search for the hidden platform for up to 90 seconds. The time taken for the animal to locate the platform was recorded as the escape latency. Animals were tested 5 times per day with a 30 second rest period between trials. Testing was completed for a total of 5 consecutive days.

Animals were divided into groups (N=12 per group) depending on treatment. Control animals received only empty vehicle. Scopolamine groups received 2 mg/kg scopolamine dissolved in sterile saline by intraperitoneal (IP) injection 30 minutes prior to testing. Test compound groups received test compound at various concentrations by oral gavage (PO) dissolved in 78% sterile saline, 20% polyethylene glycol (PEG-400), and 2% DMSO 40 minutes prior to testing. Escape latencies were recorded for each animal for 5 trials each day for 5 consecutive days. Changes in escape latency curves were statistically analyzed by 2-way ANOVA with Bonferoni post-test. Results are shown in Table 13.

TABLE 13
In Vivo Efficacy of Exemplary Compounds.

| Compound | Dose (mg/kg) | Cognitive Improvement |
|---|---|---|
| la | 8 | − |
| | 2 | − |
| | 0.5 | + |
| 2a | 10 | − |
| | 1 | +++ |
| | 0.1 | − |
| 6a | 10 | ++ |
| | 1 | − |

+++ indicates post-test p-value below 0.01
++ indicates post-test p-value between 0.05 and 0.01
+ indicates post-test p-value between 0.06 and 0.05
− indicates post-test p-value above 0.06

### Example B9: Treatment with Compound ATH-1017 Reduced a Decline in GST and Levels of Biomarkers of Neuroinflammation

Patients who had mild-to-moderate Alzheimer's disease (AD) were treated with Compound ATH-1017 or placebo as part of a randomized, double-blind, placebo-controlled, phase 2 ACT-AD study (NCT04491006). As part of this study, changes from baseline using the global statistical test (GST), a composite score informed by both ADAS-Cog11 and activities of daily living (ADCS-ADL23), were measured in participants who had mild-to-moderate Alzheimer's disease (AD) treated with Compound ATH-1017 or placebo. Changes from baseline in plasma biomarkers were also measured in these participants. The study enrolled 77 patients in the United States and Australia (age 55 to 85 years). Patients were allowed to continue standard-of-care therapy (AChEIs), with 60 percent remaining on stable doses of AChEIs and 40 percent not receiving AChEIs during the study. Patients were randomized 1:1:1 to receive placebo or Compound ATH-1017 at either 40 mg/d or 70 mg/d.

Compound ATH-1017 was generally well tolerated, with a favorable safety profile. There were no treatment related Serious Adverse Events or deaths observed in the study. Participants treated with Compound ATH-1017 at 40 mg or 70 mg for 26 weeks showed a higher incidence of treatment emergent adverse events compared to placebo. The most frequent adverse event was injection site reaction, sometimes associated with transient and asymptomatic increases in absolute Eosinophil count. The study had a 14 percent early termination rate.

The primary endpoint, ERP P300 latency, was not met by protocoled analysis. In a post-hoc analysis comparing participants receiving Compound ATH-1017 without background therapy versus placebo, 79% of patients showed improved cognition over placebo over 6 months as measured by ADAS-Cog11 in patients. 51% of showed improvement over placebo over 6 months as measured by ADCS-ADL23 in the fully study population (including patients receiving background therapy). Overall, the patients receiving Compound ATH-1017 demonstrated an improvement in GST.

Blood samples were collected at baseline (N=77) and week 26 of treatment. The biomarkers assessed included neurofilament light chain (NfL, indicates ongoing neurodegeneration), GFAP (indicates microglial activation), and YKL-40 (indicates neuroinflammation). The results are represented for pooled active arms versus placebo, without background therapy.

Baseline biomarker levels were similar between treatments groups. Participants receiving Compound ATH-1017 showed a statistically significant change from baseline versus placebo in NfL (−7.9 pg/mL, SE 2.7, p=0.0059) and directionally favorable improvements in GFAP (−29.3 pg/mL, SE 28.6, p=0.312), YKL-40 (−34.9 ng/mL, SE 26.5, p=0.195). The ApoE4 genotype, baseline Mini-Mental State Exam score, sex, or age of the participants did not affect these results. In an analysis to confirm the correlation of biomarkers and clinical endpoints, the composite endpoint GST proved to be highly correlated with changes in NfL (r=0.46, P=0.0011) and GFAP (r=0.30, P=0.0394).

The statistically significant improvement in NfL and descriptive, congruent GFAP and YKL-40 plasma concentration improvements were observed, which would be consistent with a neuroprotective effect and multimodal mechanism of action of ATH-1017. The significant correlation of NfL and GFAP reduction with the composite clinical endpoint further supports clinical relevance and suggest that Compound ATH-1017 will reduce inflammation and improve cognition in patients with long COVID.

Unless otherwise defined, all technical and scientific terms used herein have the same meaning as commonly understood by one of ordinary skill in the art to which this disclosure belongs.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

### REFERENCES

Benton, A. L., Hamsher, K., Rey, G. L., & Sivan, A. B. (1994). Multilingual Aphasia Examination (3rd ed.). Iowa City, IA: AJA Associates.
Cummings J L, Mega M, Gray K, Rosenberg-Thompson S, Carusi D A, Gornbein J. The Neuropsychiatric Inventory: comprehensive assessment of psychopathology in dementia. Neurology 1994; 44: 2308-2314.
Desole, C, Gallo, S, Vitacolonna, A, Montarolo, F, Bertolotto, A, Vivien, D, Comoglio, P, and Crepaldi, T. HGF and MET: From Brain Development to Neurological Disorders. Frontiers Cell Dev. Biol. 9:683609 (2021)
Folstein M F, Folstein S E, McHugh P R. (1975). “Mini-mental state”. A practical method for grading the cognitive state of patients for the clinician. J Psychiatr Res 12(3), 189-198.
Funakoshi, H, and Nakamura, T (2011). Hepatocyte Growth Factor (HGF): Neurotrophic functions and therapeutic implications for neuronal injury/diseases. Current Signal Transduction Therapy 6:156-167.
Galasko D, Bennett D, Sano M, Ernesto C, Thomas R, Grundman M, et al. (1997). An inventory to assess activities of daily living for clinical trials in Alzheimer's disease. The Alzheimer's disease Cooperative Study. Alzheimer's Disease and Associated Disorders, 11(S2), S33-S39.
Hamasaki, H., et al. (2014). Down-regulation of MET in hippocampal neurons of Alzheimer's disease brains. Neuropathology 34, 284-290. PMID 24444253.
Hawrylycz, M., et al. (2015). Canonical genetic signatures of the adult human brain. Nature Neuroscience 18, 1832-1844. PMID 26571460.Hyman, B. T., et al. (2012). National Institute on Aging—Alzheimer's Association guidelines on neuropathologic assessment of Alzheimer's disease. Alzheimer's & Dementia: The Journal of the Alzheimer's Association 8, 1-13.
Hilt D C, Dgetluck N, Sainati S, Moebius H J. Effect of circadian rhythm on cognition testing in a phase 2B mild/moderate Alzheimer's Disease (AD) study. J Prev Alzheimers Dis. 2015:2(4): 335.
Iadecola, C, Anrather, J, Kamel, H. Effects of COVID-19 on the Nervous System. Cell 183:16-27 (2020).
Koralnik, I J, Tyler, KL. COVID-19: A Global Threat to the Nervous System. Ann Neurol 88(1):1-11 (2020).
Magusali, N, Graham, A C, Piers, T M, Panichnantakul, P, Yaman, U, Shoai, M, Reynolds, R H, Botia, J A, Brookes, K J, Guetta-Baranes, T, Bellou, E, Bayram, S, Sokolova, D, Ryten, M, Frigerio, C S, Escott-Price, V, Morgan, K, Pocock, J M, Hardy, J, Salih, D A. A Genetic Link Between Risk for Alzheimer's Disease and Severe COVID-19 Outcomes via the OAS1 Gene. Brain doi:10.1093/brain/awab337, pages 1-15 (2021).
Marson, D C, Sawrie, S M, Snyder, S, McInturff, B, Stalvey, T, Boothe, A, Aldridge, T, Chatterjee, A, Harrell, LE. Assessing Financial Capacity in Patients with Alzheimer Disease. Arch Neurol 57:877-884 (2000).
Olichney et al. (2011) Cognitive Event-Related Potentials: Biomarkers of Synaptic Dysfunction Across the Stages of Alzheimer's Disease. Journal of Alzheimer's Disease 26:215-228.
Organ, S L, Tsao, M S (2011). An overview of the c-MET signaling pathway. Ther Adv Med Oncol 3(1):S7-S19.
Paul, B D, Lemle, M D, Komaroff, A L, Snyder, SH, Redox Imbalance Links COVID-19 and Myalgic Encephalomyelitis/Chronic Fatigue Syndrome. PNAS 118(34), e2024358118, pages 1-10 (2021).
Raahimi, M M, Kane, A, Moore, C E, Alareed, A W. Late Onset of Guillain-Barre Syndrome Following SARS-CoV-2 Infection: Part of ‘Long COVID-19 Syndrome’?BMJ Case Rep 14:e240178. doi:10.1136/bcr-2020-240178 (2021).
Raveendran, A V, Jayadeva, R, Sashidharan, S, Long COVID: An Overview. Diabetes Metab Syndr 15:869-875 (2021).
Rosen W G, Mohs R C, Davis K L (1984). A new rating Scale for Alzheimer's disease. Am J Psychiatry 141:1356-1364.
Santomauro, D, Global Prevalence and Burden of Depressive and Anxiety Disorders in 204 Countries and Territories in 2020 Due to the COVID-19 Pandemic. Lancet 398:1700-1712 (2021).
Strauss, E., Sherman, E. M. S., & Spreen, O. (2006). Compendium ofneuropsychological tests: Administration, norms, and commentary (3rd ed., p. 502). Oxford: Oxford University Press.
Thomas, A, Iacono, D, Bonanni, L, d'Andreamatteo, G, Onofrj, M, Donepezil, Rivastigmine, and Vitamin E in Alzheimer Disease: A Combined P300 Event-Related Potentials/Neuropsychologic Evaluation Over 6 Months. Clin Neuropharm 24(1):31-42 (2001).
Wang, J, Wang, P, Li, C, Huang, Y, Yang, C, Zhang, L. Central Neurological Complications and Potential Neuropathogenesis of COVID-19. Int Emerg Med Letter to the Editor, pages 1-4 (2020).

What is claimed is:

1. A method of treating long COVID, comprising administering to a patient in need thereof a therapeutically effective amount of a HGF/MET agonist.

2. A method of treating long-term sequelae of infection with SARS-CoV-2, comprising administering to a patient in need thereof a therapeutically effective amount of a HGF/MET agonist.

3. A method of treating post-acute COVID-19 syndrome, comprising administering to a patient in need thereof a therapeutically effective amount of a HGF/MET agonist.

4. A method of treating post-COVID-19 condition, comprising administering to a patient in need thereof a therapeutically effective amount of a HGF/MET agonist.

5. A method of improving event related potential (ERP) P300 latency in a patient diagnosed with long COVID, comprising administering to a patient in need thereof a therapeutically effective amount of a HGF/MET agonist.

6. A method of slowing the decline in cognition or improving cognition in a patient diagnosed with long COVID, comprising administering to a patient in need thereof a therapeutically effective amount of a HGF/MET agonist.

7. A method of slowing the decline in the ability to perform activities of daily living and verbal fluency or improving the ability to perform activities of daily living and verbal fluency in a patient diagnosed with long COVID, comprising administering to a patient in need thereof a therapeutically effective amount of a HGF/MET agonist.

8. A method of slowing the decline in functional or cognitive capacity in a patient diagnosed with long COVID, comprising administering to a patient in need thereof a therapeutically effective amount of a HGF/MET agonist.

9. A method of slowing clinical decline in a patient diagnosed with long COVID, comprising administering to a patient in need thereof a therapeutically effective amount of a HGF/MET agonist.

10. A method of improving executive memory function in a patient diagnosed with long COVID, comprising administering to a patient in need thereof a therapeutically effective amount of a HGF/MET agonist. The method of any one of the preceding claims, wherein the HGF/MET agonist is an antibody, an aptamer, a peptide, and/or a small molecule.

11. A method of treating fibrosis in a patient diagnosed with long COVID, comprising administering to a patient in need thereof a therapeutically effective amount of a HGF/MET agonist.

12. The method of claim 11, wherein the fibrosis is pulmonary fibrosis or fibrosis of peripheral nerves.

13. The method of any one of the preceding claims, comprising administering to the patient 2-90 mg per day of the HGF/MET agonist.

14. The method of any one of the preceding claims, wherein the HGF/MET agonist is N-hexanoic-Tyr-Ile-(6)aminohexanoic amide (Dihexa) or a pharmaceutically acceptable salt or a prodrug thereof.

15. The method of any one of claims 1-13, wherein the HGF/MET agonist is 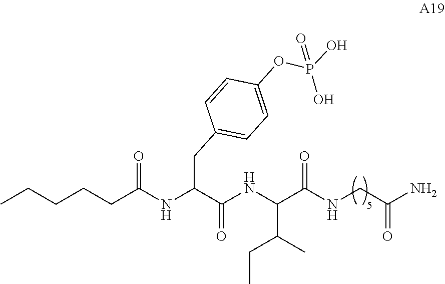
or a pharmaceutically acceptable salt thereof.

16. The method of any one of claims 1 to 13, wherein the HGF/MET agonist is a compound of Formula (I): 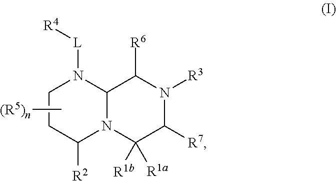
or a pharmaceutically acceptable salt thereof, wherein:
L is a direct bond, —C(═O)—, —(CR^{a}R^{b})ₘ—C(═O)—, —C(═O)—(CR^{a}R^{b})ₘ—, or —(CR^{a}R^{b})ₘ—;
each R^{a} and R^{b} is independently H, C₁-C₆ alkyl, C₂-C₆ alkenyl, or C₂-C₆ alkynyl;
R^{1a} and R^{1b} are independently H, C₁-C₆ alkyl, C₂-C₆ alkenyl, C₂-C₆ alkynyl, C₁-C₆ alkoxy, halo, or C₆-C₁₀ arylalkyl;
R² is H, oxo, or thioxo;
R³ is C₂-C₆ alkyl, C₃-C₆ alkenyl, C₃-C₆ alkynyl, C₃-C₁₂ cycloalkyl, C₃-C₆ cycloalkylalkyl, C₆-C₁₀ arylalkyl, 5- to 10-membered heteroarylalkyl, or 5- to 10-membered heterocyclylalkyl,
wherein the 5- to 10-membered heteroarylalkyl or 5- to 10-membered heterocyclylalkyl contains 1-3 heteroatoms selected from nitrogen and oxygen;
R⁴ is C₆-C₁₀ aryl, 5- to 10-membered heteroaryl, or 5- to 10-membered heterocyclyl,
wherein the 5- to 10-membered heteroaryl or 5- to 10-membered heterocyclyl contains 1-3 heteroatoms selected from nitrogen and oxygen;
each R⁵ is independently C₁-C₆ alkyl, oxo, or halo;
R⁶ is H, C₁-C₆ alkyl, or oxo;
R⁷ is H or oxo;
m is 1 or 2; and
n is an integer from 0 to 3;
wherein each C₁-C₆ alkyl, C₂-C₆ alkenyl, C₂-C₆ alkynyl, C₃-C₁₂ cycloalkyl, C₃-C₁₂ cycloalkylalkyl, C₆-C₁₀ aryl, C₆-C₁₀ arylalkyl, 5- to 10-membered heteroaryl, 5- to 10-membered heteroarylalkyl, 5- to 10-membered heterocyclyl, and 5- to 10-membered heterocyclylalkyl is optionally substituted with one to five substituents selected from hydroxyl, halo, amino, C₁-C₆ haloalkyl, C₁-C₆ alkoxy, C₁-C₆ haloalkoxy, cyano, —(C═O)NH₂, nitro, —SO₂(C₁-C₆ alkyl), and —CO₂H.

17. The method of claim 16, wherein the compound of Formula (I) is a compound of Formula (II), (IIa), (IIb), (IIc), (IId), or (IIe): 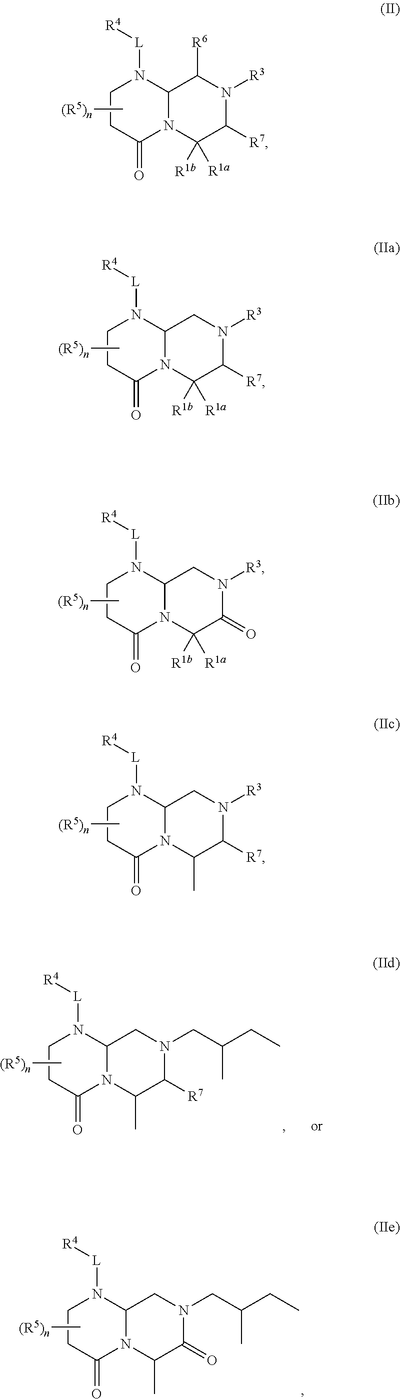
or a pharmaceutically acceptable salt thereof wherein L, R^{1a}, R^{1b}, R³, R⁴, R⁵, R⁶, R⁷, and n are as described for Formula (I).

18. The method of any one of claims 1 to 13 or 16 to 17, wherein the HGF/MET agonist is a Compound as set forth in Table 1, or a pharmaceutically acceptable salt thereof or a Compound as set forth in Table 1A, or a pharmaceutically acceptable salt thereof.

19. The method of any one of claims 1 to 13 or 16 to 18, wherein the HGF/MET agonist is 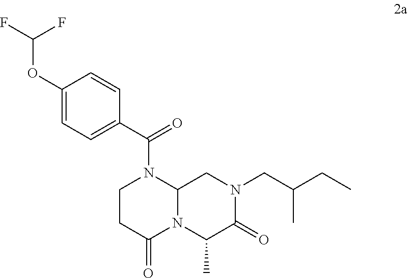 [SMILES: CCC(C)CN1CC2N(C(=O)c3ccc(OC(F)F)cc3)CCC(=O)N2[C@@H](C)C1=O]
or a pharmaceutically acceptable salt thereof.

20. The method of any one of claims 1 to 13 or 16 to 18, wherein the HGF/MET agonist is 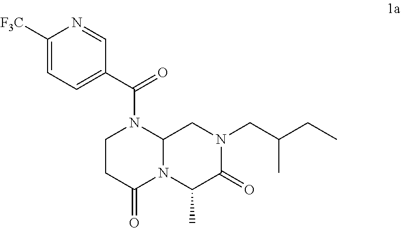 [SMILES: CCC(C)CN1CC2N(C(=O)c3ccc(C(F)(F)F)nc3)CCC(=O)N2[C@@H](C)C1=O]
or a pharmaceutically acceptable salt thereof.

21. The method of any one of the preceding claims, wherein prior to treatment, the patient exhibits one or more symptoms selected from anxiety, apathy, breathlessness, chest pain, cognitive impairment, depression, fatigue, impaired lung function, reduced liver function, increased rate of venous thromboembolism, muscle weakness, fibrosis, pulmonary fibrosis, idiopathic pulmonary fibrosis, fibrosis of peripheral nerves, and/or respiratory complications.

22. The method of any one of the preceding claims, wherein prior to treatment, the patient exhibits cognitive impairment.

23. The method of claim 22, wherein the cognitive impairment is an attention, working memory, processing speed, executive functioning, phonemic fluency, category fluency, memory encoding, memory recall, and/or memory recognition cognitive impairment.

24. The method of claim 22 or claim 23, wherein the cognitive impairment suggests an executive pattern.

25. The method of any one of claims 22 to 24, wherein the cognitive impairment suggests a dysexecutive syndrome.

26. The method of any one of the preceding claims, wherein prior to treatment, the patient exhibits fibrosis.

27. The method of claim 26, wherein the fibrosis is a pulmonary fibrosis and/or fibrosis of peripheral nerves.

28. The method of any one of the preceding claims, wherein the patient has previously been diagnosed with airways disease, asthma such as moderate to severe asthma, bronchiectasis, bronchopulmonary dysplasia, cancer, chronic lung disease, chronic obstructive pulmonary disease (COPD), diabetes, hypertension, interstitial lung disease, pulmonary embolism, and/or pulmonary hypertension.

29. The method of any one of the preceding claims, which slows the decline in functional or cognitive capacity in the patient.

30. The method of any one of the preceding claims, which slows the decline in cognition in the patient.

31. The method of any one of the preceding claims, which improves cognition in the patient.

32. The method of any one of the preceding claims, which slows the decline in the ability to perform instrumental activities of daily living, basic activities of daily living, financial capacity, and/or verbal fluency in the patient.

33. The method of any one of the preceding claims, which improves the ability to perform activities of daily living, financial capacity, and/or verbal fluency in the patient.

34. The method of any one of the preceding claims, wherein the slowing of the decline or the improvement is determined after administering the treatment for at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks.

35. The method of any one of the preceding claims, which reduces the rate of decline, stabilizes, or improves an Instrumental (23-item) Activities of Daily Living (ADL) score.

36. The method of claim 35, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

37. The method of claim 35 or claim 36, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

38. The method of any one of the preceding claims, which reduces the rate of decline, stabilizes, or improves a Basic (19-item) Activities of Daily Living (ADL) score.

39. The method of claim 38, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

40. The method of claim 38 or claim 39, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

41. The method of any one of the preceding claims, which reduces the rate of decline, stabilizes, or improves a Financial Capacity Instrument (FCI) score.

42. The method of claim 41, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

43. The method of claim 41 or claim 42, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

44. The method of any one of the preceding claims, wherein the patient has a Mini-Mental State Examination (MMSE) score of less than or equal to 27 prior to the start of treatment.

45. The method of any one of the preceding claims, which reduces the rate of decline, stabilizes, or improves an MMSE score.

46. The method of claim 45, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

47. The method of claim 45 or claim 46, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

48. The method of any one of the preceding claims, wherein cognitive capacity is assessed by determining the patient's score before and after administration of the treatment using an Alzheimer's Disease (AD) Assessment Scale-Cognitive Subscale (ADAS-Cog).

49. The method of claim 48, wherein cognitive capacity is assessed prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

50. The method of claim 48 or claim 49, which reduces the rate of decline, stabilizes, or improves ADAS-Cog.

51. The method of any one of the preceding claims, which reduces the rate of decline, stabilizes, or improves a Montreal Cognitive Assessment test (MoCA) score.

52. The method of claim 51, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

53. The method of claim 51 or claim 52, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

54. The method of any one of the preceding claims, which reduces the rate of decline, stabilizes, or improves a Digit Symbol Substitution Test (DSST) score.

55. The method of claim 54, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

56. The method of claim 54 or claim 55, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

57. The method of any one of the preceding claims, which reduces the rate of decline, stabilizes, or improves a Disability Assessment for Dementia (DAD) score.

58. The method of claim 57, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

59. The method of claim 57 or claim 58, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

60. The method of any one of the preceding claims, which reduces the rate of decline, stabilizes, or improves a neuropsychiatric inventory (NPI) score.

61. The method of claim 60, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

62. The method of claim 60 or claim 61, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

63. The method of any one of the preceding claims, which reduces the rate of decline, stabilizes, or improves a Controlled Oral Word Association Test (COWAT) score.

64. The method of claim 63, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

65. The method of claim 63 or claim 64, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

66. The method of any one of the preceding claims, which reduces the rate of decline, stabilizes, or improves a Number Span forward and/or backward score.

67. The method of claim 66, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

68. The method of claim 66 or claim 67, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

69. The method of any one of the preceding claims, which reduces the rate of decline, stabilizes, or improves a Trail Making Test Part A and/or Part B score.

70. The method of claim 69, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

71. The method of claim 69 or claim 70, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

72. The method of any one of the preceding claims, which reduces the rate of decline, stabilizes, or improves a phonemic and category fluency score.

73. The method of claim 72, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

74. The method of claim 72 or claim 73, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

75. The method of any one of the preceding claims, which reduces the rate of decline, stabilizes, or improves a Hopkins Verbal Learning Test-Revised score.

76. The method of claim 75, wherein the reduction in the rate of decline, stabilization, or improvement occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

77. The method of claim 75 or claim 76, wherein reducing the rate of decline, stabilizing, or improving is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

78. The method of any one of the preceding claims, which reduces, stabilizes, or slows the development of fibrosis.

79. The method of claim 78, wherein the fibrosis is pulmonary fibrosis and/or fibrosis of peripheral nerves.

80. The method of claim 78 or claim 79, wherein the reduction, stabilization, or slowing development occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

81. The method of any one of claims 78-80, wherein the reduction, stabilization, or slowing development is assessed by determining the patient's score prior to the start of treatment and at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

82. The method of any one of the preceding claims, which provides fast improvement or normalization of P300 amplitude values.

83. The method of any one of the preceding claims, which provides fast improvement or normalization of event-related potential (ERP) P300 latency values.

84. The method of any one of the preceding claims, which provides fast improvement or normalization of ERP P300 latency values with some maintenance of effect at 4 weeks after discontinuation of treatment.

85. The method of any one of the preceding claims, which provides fast improvement or normalization of ERP P300 latency values, which is maintained at 4 weeks after discontinuation of treatment.

86. The method of any one of the preceding claims, which improves event-related potential (ERP) P300 latency.

87. The method of any one of claims 82 to 86, wherein the improvement or normalization of P300 values occurs by at least 2 weeks, at least 4 weeks, at least 6 weeks, at least 8 weeks, at least 10 weeks, at least 12 weeks, at least 16 weeks, at least 18 weeks, at least 20 weeks, at least 22 weeks, at least 24 weeks, or at least 26 weeks after the start of treatment.

88. The method of any one of the preceding claims, wherein the HGF/MET agonist has an acceptable safety and tolerability profile.

89. The method of any one of the preceding claims, wherein the HGF/MET agonist is generally safe and well tolerated.

90. The method of any one of the preceding claims, wherein the HGF/MET agonist is administered by subcutaneous injection or by oral dosage, such as by solid oral dosage form.

91. The method of any one of the preceding claims, wherein the HGF/MET agonist is administered by subcutaneous injection.

92. The method of any one of the preceding claims, comprising administering the HGF/MET agonist for 26 weeks or more.

93. The method of any one of the preceding claims, wherein the HGF/MET agonist is a sodium salt of the compound of formula A19.

94. The method of any one of the preceding claims, wherein the HGF/MET agonist is a monosodium salt of the compound of formula A19.
